SEQUENCE LISTING (492 sequences)

SEQ ID NO: 1 - protein, 15 aa, synthetic construct
GGSCWIPFEWIDCGG

SEQ ID NO: 2 - protein, 19 aa, synthetic construct
GGICWVPFHYLDELMCTGG

SEQ ID NO: 3 - protein, 18 aa, synthetic construct
GGDQCWQPFQYVACPPGG

SEQ ID NO: 4 - protein, 18 aa, synthetic construct
GGEECWLPYHFVRCDDGG

SEQ ID NO: 5 - protein, 18 aa, synthetic construct
GGDGCWVPFHFVNCQIGG

SEQ ID NO: 6 - protein, 18 aa, synthetic construct
GGPLCWEPFTYVSCESGG

SEQ ID NO: 7 - protein, 18 aa, synthetic construct
GGQKCWRPFTYFDCLEGG

SEQ ID NO: 8 - protein, 18 aa, synthetic construct
GGQTCWEPFRYIDCGEGG

SEQ ID NO: 9 - protein, 18 aa, synthetic construct
GGQLCWAPFSFYKCDSGG

SEQ ID NO: 10 - protein, 18 aa, synthetic construct
GGELCWRPFGYYACEQGG

SEQ ID NO: 11 - protein, 18 aa, synthetic construct
GGGLCWVPFMYVTCEKGG

SEQ ID NO: 12 - protein, 18 aa, synthetic construct
GGRACWRPFNFFDCVIGG

SEQ ID NO: 13 - protein, 18 aa, synthetic construct
GGTICWKPFSFYQCHEGG

SEQ ID NO: 14 - protein, 18 aa, synthetic construct
GGPLCWKPFSFYACESGG

SEQ ID NO: 15 - protein, 18 aa, synthetic construct
GGELCWRPFSFVDCEEGG

SEQ ID NO: 16 - protein, 11 aa, synthetic construct
SCWIPFEWIDC

SEQ ID NO: 17 - protein, 15 aa, synthetic construct
ICWVPFHYLDELMCT

SEQ ID NO: 18 - protein, 14 aa, synthetic construct
DQCWQPFQYVACPP

SEQ ID NO: 19 - protein, 14 aa, synthetic construct
EECWLPYHFVRCDD

SEQ ID NO: 20 - protein, 14 aa, synthetic construct
DGCWVPFHFVNCQI

SEQ ID NO: 21 - protein, 14 aa, synthetic construct
PLCWEPFTYVSCES

SEQ ID NO: 22 - protein, 14 aa, synthetic construct
QKCWRPFTYFDCLE

SEQ ID NO: 23 - protein, 14 aa, synthetic construct
QTCWEPFRYIDCGE

SEQ ID NO: 24 - protein, 14 aa, synthetic construct
QLCWAPFSFYKCDS

SEQ ID NO: 25 - protein, 14 aa, synthetic construct
ELCWRPFGYYACEQ

SEQ ID NO: 26 - protein, 14 aa, synthetic construct
GLCWVPFMYVTCEK

SEQ ID NO: 27 - protein, 14 aa, synthetic construct
RACWRPFNFFDCVI

SEQ ID NO: 28 - protein, 14 aa, synthetic construct
TICWKPFSFYQCHE

SEQ ID NO: 29 - protein, 14 aa, synthetic construct
PLCWKPFSFYACES

SEQ ID NO: 30 - protein, 14 aa, synthetic construct
ELCWRPFSFVDCEE

SEQ ID NO: 31 - protein, 24 aa, synthetic construct
GGLSPICWVPFHYVDYWMCNLEGG

SEQ ID NO: 32 - protein, 24 aa, synthetic construct
GGLSSICWVPFHYVDEAACQLGGG

SEQ ID NO: 33 - protein, 24 aa, synthetic construct
GGYLDTCWVPFHYVNYLMCNVEGG

SEQ ID NO: 34 - protein, 24 aa, synthetic construct
GGDSEVCWVPFHYINHLMCTHDGG

SEQ ID NO: 35 - protein, 24 aa, synthetic construct
GGNANICWVPFHYVNETMCNVGGG

SEQ ID NO: 36 - protein, 24 aa, synthetic construct
GGNETLCWVPFHYVDYLFCNHDGG

SEQ ID NO: 37 - protein, 24 aa, synthetic construct
GGLESPCWVPFHFLDERMCMSGGG

SEQ ID NO: 38 - protein, 24 aa, synthetic construct
GGDSSMCWVPFHYVDEFMCNEDGG

SEQ ID NO: 39 - protein, 24 aa, synthetic construct
GGSKQICWVPFHYVDEQFCNDRGG

SEQ ID NO: 40 - protein, 24 aa, synthetic construct
GGYSSICWVPFHYVDERACTGQGG

SEQ ID NO: 41 - protein, 24 aa, synthetic construct
GGENMNCWVPFHYIDYLLCEDVGG

SEQ ID NO: 42 - protein, 20 aa, synthetic construct
LSPICWVPFHYVDYWMCNLE

SEQ ID NO: 43 - protein, 20 aa, synthetic construct
LSSICWVPFHYVDEAACQLG

SEQ ID NO: 44 - protein, 20 aa, synthetic construct
YLDTCWVPFHYVNYLMCNVE

SEQ ID NO: 45 - protein, 20 aa, synthetic construct
DSEVCWVPFHYINHLMCTHD

SEQ ID NO: 46 - protein, 20 aa, synthetic construct
NANICWVPFHYVNETMCNVG

SEQ ID NO: 47 - protein, 20 aa, synthetic construct
NETLCWVPFHYVDYLFCNHD

SEQ ID NO: 48 - protein, 20 aa, synthetic construct
LESPCWVPFHFLDERMCMSG

SEQ ID NO: 49 - protein, 20 aa, synthetic construct
DSSMCWVPFHYVDEFMCNED

SEQ ID NO: 50 - protein, 20 aa, synthetic construct
SKQICWVPFHYVDEQFCNDR

SEQ ID NO: 51 - protein, 20 aa, synthetic construct
YSSICWVPFHYVDERACTGQ

SEQ ID NO: 52 - protein, 20 aa, synthetic construct
ENMNCWVPFHYIDYLLCEDV

SEQ ID NO: 53 - protein, 24 aa, synthetic construct
GGHDLLCFRPWEDVEDLVCTEDGG

SEQ ID NO: 54 - protein, 24 aa, synthetic construct
GGERLMCSVPWEDVADFICMGDGG

SEQ ID NO: 55 - protein, 24 aa, synthetic construct
GGVQMICWLPWPDVDDHICTEVGG

SEQ ID NO: 56 - protein, 24 aa, synthetic construct
GGNHRICWLPWEDVDDRICISRGG

SEQ ID NO: 57 - protein, 24 aa, synthetic construct
GGLYSMCFRPWEDVEDFICTTNGG

SEQ ID NO: 58 - protein, 24 aa, synthetic construct
GGRSTLCWVPWEGLGDRICTAQGG

SEQ ID NO: 59 - protein, 24 aa, synthetic construct
GGSYLTCWVPWQGLDDRFCTETGG

SEQ ID NO: 60 - protein, 24 aa, synthetic construct
GGTDYTCFVPWNDVFDRVCTWSGG

SEQ ID NO: 61 - protein, 24 aa, synthetic construct
GGSHLICWMPWPEVEDRLCTDSGG

SEQ ID NO: 62 - protein, 24 aa, synthetic construct
GGTKLMCWVPWQDVDDFICTSLGG

SEQ ID NO: 63 - protein, 24 aa, synthetic construct
GGFYQSCWHPWEDIDDHICTYSGG

SEQ ID NO: 64 - protein, 24 aa, synthetic construct
GGFKLSCWVPWQDTEDRICWTVGG

SEQ ID NO: 65 - protein, 24 aa, synthetic construct
GGSHWICWWPFSDTEDHICSEYGG

SEQ ID NO: 66 - protein, 24 aa, synthetic construct
GGSRLVCWVPFEDFMDSICTEYGG

SEQ ID NO: 67 - protein, 24 aa, synthetic construct
GGDTYTCFFPWPGLDDHFCTYSGG

SEQ ID NO: 68 - protein, 20 aa, synthetic construct
HDLLCFRPWEDVEDLVCTED

SEQ ID NO: 69 - protein, 20 aa, synthetic construct
ERLMCSVPWEDVADFICMGD

SEQ ID NO: 70 - protein, 20 aa, synthetic construct
VQMICWLPWPDVDDHICTEV

SEQ ID NO: 71 - protein, 20 aa, synthetic construct
NHRICWLPWEDVDDRICISR

SEQ ID NO: 72 - protein, 20 aa, synthetic construct
LYSMCFRPWEDVEDFICTTN

SEQ ID NO: 73 - protein, 20 aa, synthetic construct
RSTLCWVPWEGLGDRICTAQ

SEQ ID NO: 74 - protein, 20 aa, synthetic construct
SYLTCWVPWQGLDDRFCTET

SEQ ID NO: 75 - protein, 20 aa, synthetic construct
TDYTCFVPWNDVFDRVCTWS

SEQ ID NO: 76 - protein, 20 aa, synthetic construct
SHLICWMPWPEVEDRLCTDS

SEQ ID NO: 77 - protein, 20 aa, synthetic construct
TKLMCWVPWQDVDDFICTSL

SEQ ID NO: 78 - protein, 20 aa, synthetic construct
FYQSCWHPWEDIDDHICTYS

SEQ ID NO: 79 - protein, 20 aa, synthetic construct
FKLSCWVPWQDTEDRICWTV

SEQ ID NO: 80 - protein, 20 aa, synthetic construct
SHWICWWPFSDTEDHICSEY

SEQ ID NO: 81 - protein, 20 aa, synthetic construct
SRLVCWVPFEDFMDSICTEY

SEQ ID NO: 82 - protein, 20 aa, synthetic construct
DTYTCFFPWPGLDDHFCTYS

SEQ ID NO: 83 - protein, 20 aa, synthetic construct
GGFNVCDFDPGWWDCLQLGG

SEQ ID NO: 84 - protein, 20 aa, synthetic construct
GGFNACSFEPGWWGCEVYGG

SEQ ID NO: 85 - protein, 20 aa, synthetic construct
GGSWLCSLEPGWWECNPGGG

SEQ ID NO: 86 - protein, 16 aa, synthetic construct
FNVCDFDPGWWDCLQL

SEQ ID NO: 87 - protein, 16 aa, synthetic construct
FNACSFEPGWWGCEVY

SEQ ID NO: 88 - protein, 16 aa, synthetic construct
SWLCSLEPGWWECNPG

SEQ ID NO: 89 - protein, 20 aa, synthetic construct
GGSLVCDYLPGMSLCYESGG

SEQ ID NO: 90 - protein, 16 aa, synthetic construct
SLVCDYLPGMSLCYES

SEQ ID NO: 91 - protein, 18 aa, synthetic construct
GGCELVWTPFQFVEHCGG

SEQ ID NO: 92 - protein, 18 aa, synthetic construct
GGCSLEWVPFTYVEECGG

SEQ ID NO: 93 - protein, 14 aa, synthetic construct
CELVWTPFQFVEHC

SEQ ID NO: 94 - protein, 14 aa, synthetic construct
CSLEWVPFTYVEEC

SEQ ID NO: 95 - protein, 18 aa, synthetic construct
GGCSRWVPFHYVVGDCGG

SEQ ID NO: 96 - protein, 14 aa, synthetic construct
CSRWVPFHYVVGDC

SEQ ID NO: 97 - protein, 24 aa, synthetic construct
GGANYLECFYSPTQEWCIPHLMGG

SEQ ID NO: 98 - protein, 23 aa, synthetic construct
GGESFLECFYSPAEWCIPSVIGG

SEQ ID NO: 99 - protein, 24 aa, synthetic construct
GGFVFMECFWSPSQEWCIPSFTGG

SEQ ID NO: 100 - protein, 24 aa, synthetic construct
GGGVSFDCFWSPGQYWCIPDYNGG

SEQ ID NO: 101 - protein, 24 aa, synthetic construct
GGLDVMECFWSPSQVWCMPSVFGG

SEQ ID NO: 102 - protein, 24 aa, synthetic construct
GGLYHFECAWSPYQHWCWPSQDGG

SEQ ID NO: 103 - protein, 24 aa, synthetic construct
GGREDMECFWSPGQVWCIPSYLGG

SEQ ID NO: 104 - protein, 24 aa, synthetic construct
GGREKLECFWSPGQEWCIPILQGG

SEQ ID NO: 105 - protein, 24 aa, synthetic construct
GGRGFLECFYSPGQEWCIPSIWGG

SEQ ID NO: 106 - protein, 24 aa, synthetic construct
GGSALLECFYSPTQEWCIPTLWGG

SEQ ID NO: 107 - protein, 24 aa, synthetic construct
GGSSYSECFWSPGQLWCIPSFPGG

SEQ ID NO: 108 - protein, 24 aa, synthetic construct
GGVSIFECFYSPTQEWCIPSFIGG

SEQ ID NO: 109 - protein, 24 aa, synthetic construct
GGWDILECFYSPGQEWCIPSFLGG

SEQ ID NO: 110 - protein, 24 aa, synthetic construct
GGYGSWECYWSPSEEWCIPAQLGG

SEQ ID NO: 111 - protein, 20 aa, synthetic construct
ANYLECFYSPTQEWCIPHLM

SEQ ID NO: 112 - protein, 19 aa, synthetic construct
ESFLECFYSPAEWCIPSVI

SEQ ID NO: 113 - protein, 20 aa, synthetic construct
FVFMECFWSPSQEWCIPSFT

SEQ ID NO: 114 - protein, 20 aa, synthetic construct
GVSFDCFWSPGQYWCIPDYN

SEQ ID NO: 115 - protein, 20 aa, synthetic construct
LDVMECFWSPSQVWCMPSVF

SEQ ID NO: 116 - protein, 20 aa, synthetic construct
LYHFECAWSPYQHWCWPSQD

SEQ ID NO: 117 - protein, 20 aa, synthetic construct
REDMECFWSPGQVWCIPSYL

SEQ ID NO: 118 - protein, 20 aa, synthetic construct
REKLECFWSPGQEWCIPILQ

SEQ ID NO: 119 - protein, 20 aa, synthetic construct
RGFLECFYSPGQEWCIPSIW

SEQ ID NO: 120 - protein, 20 aa, synthetic construct
SALLECFYSPTQEWCIPTLW

SEQ ID NO: 121 - protein, 20 aa, synthetic construct
SSYSECFWSPGQLWCIPSFP

SEQ ID NO: 122 - protein, 20 aa, synthetic construct
VSIFECFYSPTQEWCIPSFI

SEQ ID NO: 123 - protein, 20 aa, synthetic construct
WDILECFYSPGQEWCIPSFL

SEQ ID NO: 124 - protein, 20 aa, synthetic construct
YGSWECYWSPSEEWCIPAQL

SEQ ID NO: 125 - protein, 19 aa, synthetic construct
GGWSQCLWEPLWECWTDGG

SEQ ID NO: 126 - protein, 20 aa, synthetic construct
GGFFECFYSPGHEWCIPSGG

SEQ ID NO: 127 - protein, 16 aa, synthetic construct
FFECFYSPGHEWCIPS

SEQ ID NO: 128 - protein, 18 aa, synthetic construct
GGMECWYSPHEVWCSTGG

SEQ ID NO: 129 - protein, 14 aa, synthetic construct
MECWYSPHEVWCST

SEQ ID NO: 130 - protein, 20 aa, synthetic construct
GGPYCESMEPGQYTECTWGG

SEQ ID NO: 131 - protein, 20 aa, synthetic construct
GGLVCYGDSPGDDYVCIEGG

SEQ ID NO: 132 - protein, 20 aa, synthetic construct
GGWECWPDNPGIYWNCQPGG

SEQ ID NO: 133 - protein, 20 aa, synthetic construct
GGRNCYTMPPGIYKECAWGG

SEQ ID NO: 134 - protein, 20 aa, synthetic construct
GGFSCWPQEPGIYYNCLVGG

SEQ ID NO: 135 - protein, 20 aa, synthetic construct
GGPSCWPQEPGIYYNCIYGG

SEQ ID NO: 136 - protein, 16 aa, synthetic construct
PYCESMEPGQYTECTW

SEQ ID NO: 137 - protein, 16 aa, synthetic construct
LVCYGDSPGDDYVCIE

SEQ ID NO: 138 - protein, 16 aa, synthetic construct
WECWPDNPGIYWNCQP

SEQ ID NO: 139 - protein, 16 aa, synthetic construct
RNCYTMPPGIYKECAW

SEQ ID NO: 140 - protein, 16 aa, synthetic construct
FSCWPQEPGIYYNCLV

SEQ ID NO: 141 - protein, 16 aa, synthetic construct
PSCWPQEPGIYYNCIY

SEQ ID NO: 142 - protein, 18 aa, synthetic construct
GGCNYKEYQDGIWMVCGG

SEQ ID NO: 143 - protein, 20 aa, synthetic construct
GGVFTCPPPPGENHCGVFGG

SEQ ID NO: 144 - protein, 18 aa, synthetic construct
GGDCWDQNTFRYIDCFGG

SEQ ID NO: 145 - protein, 16 aa, synthetic construct
GGRSDGFVWWLGWLGG

SEQ ID NO: 146 - protein, 16 aa, synthetic construct
GGMFEAEWFLEQFGGG

SEQ ID NO: 147 - protein, 18 aa, synthetic construct
GGYRFLCQDGFCLQWSGG

SEQ ID NO: 148 - protein, 20 aa, synthetic construct
GGASFCDILPGMEWCTYDGG

SEQ ID NO: 149 - protein, 19 aa, synthetic construct
GGQACTRNFWGLYRCNAGG

SEQ ID NO: 150 - protein, 18 aa, synthetic construct
GGLICDMQDGMSFCYDGG

SEQ ID NO: 151 - protein, 18 aa, synthetic construct
GGCYWIEKESTWHVECGG

SEQ ID NO: 152 - protein, 14 aa, synthetic construct
CNYKEYQDGIWMVC

SEQ ID NO: 153 - protein, 16 aa, synthetic construct
VFTCPPPPGENHCGVF

SEQ ID NO: 154 - protein, 14 aa, synthetic construct
DCWDQNTFRYIDCF

SEQ ID NO: 155 - protein, 12 aa, synthetic construct
RSDGFVWWLGWL

SEQ ID NO: 156 - protein, 12 aa, synthetic construct
MFEAEWFLEQFG

SEQ ID NO: 157 - protein, 14 aa, synthetic construct
YRFLCQDGFCLQWS

SEQ ID NO: 158 - protein, 16 aa, synthetic construct
ASFCDILPGMEWCTYD

SEQ ID NO: 159 - protein, 15 aa, synthetic construct
QACTRNFWGLYRCNA

SEQ ID NO: 160 - protein, 14 aa, synthetic construct
LICDMQDGMSFCYD

SEQ ID NO: 161 - protein, 14 aa, synthetic construct
CYWIEKESTWHVEC

SEQ ID NO: 162 - protein, 22 aa, synthetic construct
REDMECFWSPGQVWCIPSYLGG

SEQ ID NO: 163 - protein, 21 aa, synthetic construct
EDMECFWSPGQVWCIPSYLGG

SEQ ID NO: 164 - protein, 20 aa, synthetic construct
DMECFWSPGQVWCIPSYLGG

SEQ ID NO: 165 - protein, 19 aa, synthetic construct
MECFWSPGQVWCIPSYLGG

SEQ ID NO: 166 - protein, 18 aa, synthetic construct
ECFWSPGQVWCIPSYLGG

SEQ ID NO: 167 - protein, 22 aa, synthetic construct
GGREDMECFWSPGQVWCIPSYL

SEQ ID NO: 168 - protein, 21 aa, synthetic construct
GGREDMECFWSPGQVWCIPSY

SEQ ID NO: 169 - protein, 20 aa, synthetic construct
GGREDMECFWSPGQVWCIPS

SEQ ID NO: 170 - protein, 19 aa, synthetic construct
EDMECFWSPGQVWCIPSYL

SEQ ID NO: 171 - protein, 18 aa, synthetic construct
DMECFWSPGQVWCIPSYL

SEQ ID NO: 172 - protein, 17 aa, synthetic construct
MECFWSPGQVWCIPSYL

SEQ ID NO: 173 - protein, 16 aa, synthetic construct
ECFWSPGQVWCIPSYL

SEQ ID NO: 174 - protein, 19 aa, synthetic construct
REDMECFWSPGQVWCIPSY

SEQ ID NO: 175 - protein, 18 aa, synthetic construct
REDMECFWSPGQVWCIPS

SEQ ID NO: 176 - protein, 24 aa, synthetic construct
GGQTFLHCFESRGIDWCIPWKSGG

SEQ ID NO: 177 - protein, 20 aa, synthetic construct
QTFLHCFESRGIDWCIPWKS

SEQ ID NO: 178 - protein, 18 aa, synthetic construct
GGHCFWVNDMMEVVCDGG

SEQ ID NO: 179 - protein, 14 aa, synthetic construct
HCFWVNDMMEVVCD

SEQ ID NO: 180 - protein, 20 aa, synthetic construct
GGFTCWQDYPGDTVYCYPGG

SEQ ID NO: 181 - protein, 20 aa, synthetic construct
GGQECWISDSGTYLWCTQGG

SEQ ID NO: 182 - protein, 20 aa, synthetic construct
GGWECWQDLPGSSIVCTDGG

SEQ ID NO: 183 - protein, 16 aa, synthetic construct
FTCWQDYPGDTVYCYP

SEQ ID NO: 184 - protein, 16 aa, synthetic construct
QECWISDSGTYLWCTQ

SEQ ID NO: 185 - protein, 16 aa, synthetic construct
WECWQDLPGSSIVCTD

SEQ ID NO: 186 - protein, 20 aa, synthetic construct
GGDFCEPLWPGDNTPCIVGG

SEQ ID NO: 187 - protein, 20 aa, synthetic construct
GGHNCVPLWPGDTSPCYDGG

SEQ ID NO: 188 - protein, 20 aa, synthetic construct
GGQDCYPLWPGEEISCETGG

SEQ ID NO: 189 - protein, 20 aa, synthetic construct
GGLDCLPLWPGDADWCLAGG

SEQ ID NO: 190 - protein, 20 aa, synthetic construct
GGDSCWVLWPGDADWCTIGG

SEQ ID NO: 191 - protein, 16 aa, synthetic construct
DFCEPLWPGDNTPCIV

SEQ ID NO: 192 - protein, 16 aa, synthetic construct
HNCVPLWPGDTSPCYD

SEQ ID NO: 193 - protein, 16 aa, synthetic construct
QDCYPLWPGEEISCET

SEQ ID NO: 194 - protein, 16 aa, synthetic construct
LDCLPLWPGDADWCLA

SEQ ID NO: 195 - protein, 16 aa, synthetic construct
DSCWVLWPGDADWCTI

SEQ ID NO: 196 - protein, 19 aa, synthetic construct
GGFCRTFLSLLSFIHCHGG

SEQ ID NO: 197 - protein, 15 aa, synthetic construct
FCRTFLSLLSFIHCH

SEQ ID NO: 198 - protein, 18 aa, synthetic construct
GGCNDYVNRLWELPDCGG

SEQ ID NO: 199 - protein, 18 aa, synthetic construct
GGGCLSFTPRVWMFCWGG

SEQ ID NO: 200 - protein, 14 aa, synthetic construct
CNDYVNRLWELPDC

SEQ ID NO: 201 - protein, 19 aa, synthetic construct
GGRFCQVLDNGVHACLIGG

SEQ ID NO: 202 - protein, 15 aa, synthetic construct
RFCQVLDNGVHACLI

SEQ ID NO: 203 - protein, 19 aa, synthetic construct
GGPYCLWGSGTYFDCMRGG

SEQ ID NO: 204 - protein, 19 aa, synthetic construct
GGKFCSFGAMSLPYCRLGG

SEQ ID NO: 205 - protein, 15 aa, synthetic construct
PYCLWGSGTYFDCMR

SEQ ID NO: 206 - protein, 15 aa, synthetic construct
KFCSFGAMSLPYCRL

SEQ ID NO: 207 - protein, 18 aa, synthetic construct
GGYTCMNDHPEILCLTGG

SEQ ID NO: 208 - protein, 18 aa, synthetic construct
GGKVCDYVTSNGICMVGG

SEQ ID NO: 209 - protein, 18 aa, synthetic construct
GGYHCDWIDESGVCIVGG

SEQ ID NO: 210 - protein, 18 aa, synthetic construct
GGFPCAFRAPWAECSIGG

SEQ ID NO: 211 - protein, 18 aa, synthetic construct
GGKVCHWEGQMLLCRLGG

SEQ ID NO: 212 - protein, 14 aa, synthetic construct
YTCMNDHPEILCLT

SEQ ID NO: 213 - protein, 14 aa, synthetic construct
KVCDYVTSNGICMV

SEQ ID NO: 214 - protein, 14 aa, synthetic construct
YHCDWIDESGVCIV

SEQ ID NO: 215 - protein, 14 aa, synthetic construct
FPCAFRAPWAECSI

SEQ ID NO: 216 - protein, 14 aa, synthetic construct
KVCHWEGQMLLCRL

SEQ ID NO: 217 - protein, 18 aa, synthetic construct
GGLCWYFGGTVRGPCLGG

SEQ ID NO: 218 - protein, 14 aa, synthetic construct
LCWYFGGTVRGPCL

SEQ ID NO: 219 - protein, 24 aa, synthetic construct
GGEGFFCEYWGVTGLGPCPGINGG

SEQ ID NO: 220 - protein, 24 aa, synthetic construct
GGIYGLCSYFGGTVWGLCLGDSGG

SEQ ID NO: 221 - protein, 24 aa, synthetic construct
GGGASQCWYFGGTVRGPCLGPIGG

SEQ ID NO: 222 - protein, 24 aa, synthetic construct
GGKGHGCWYLGGTGQGPCLGGKGG

SEQ ID NO: 223 - protein, 24 aa, synthetic construct
GGGDYLCWYFGGTVRGSCAGTTGG

SEQ ID NO: 224 - protein, 24 aa, synthetic construct
GGTSRLCWYFAGTVPGPCLEAIGG

SEQ ID NO: 225 - protein, 24 aa, synthetic construct
GGNNFMCWYFGGTVRAPCLQYMGG

SEQ ID NO: 226 - protein, 24 aa, synthetic construct
GGGSGICWYFAGTVPGPCLSQTGG

SEQ ID NO: 227 - protein, 24 aa, synthetic construct
GGGEKLCWYFGETVRGPCLKWMGG

SEQ ID NO: 228 - protein, 24 aa, synthetic construct
GGSSRLCWYFAGTVQGPCLSSLGG

SEQ ID NO: 229 - protein, 24 aa, synthetic construct
GGGEKLCWFFGGTVRAPCLNHMGG

SEQ ID NO: 230 - protein, 24 aa, synthetic construct
GGKDRRCWYFGGTVQGPCLSSNGG

SEQ ID NO: 231 - protein, 24 aa, synthetic construct
GGAAAPCWYFGGTVRGPCLGARGG

SEQ ID NO: 232 - protein, 24 aa, synthetic construct
GGFSPLCYYFGGTVRGPCLGGTGG

SEQ ID NO: 233 - protein, 24 aa, synthetic construct
GGKGHLCSYFAGTVLGPCLWDTGG

SEQ ID NO: 234 - protein, 24 aa, synthetic construct
GGPVRLCWYFQETVRAPCLKFMGG

SEQ ID NO: 235 - protein, 24 aa, synthetic construct
GGGQYLCWYFGGAVSGTCLDDYGG

SEQ ID NO: 236 - protein, 24 aa, synthetic construct
GGSGVLCWYFGGRVPGPCLGQNGG

SEQ ID NO: 237 - protein, 24 aa, synthetic construct
GGREKLCVYFGGTVWGPCLDGTGG

SEQ ID NO: 238 - protein, 24 aa, synthetic construct
GGGKELCWYFAGTVRGPCLGSIGG

SEQ ID NO: 239 - protein, 24 aa, synthetic construct
GGWWGLCSYFGGTVSGPCLTERGG

SEQ ID NO: 240 - protein, 24 aa, synthetic construct
GGDSRLCWYFAGTVLGPCLHQIGG

SEQ ID NO: 241 - protein, 24 aa, synthetic construct
GGLTGLCSYFGGSVQGPCLAQSGG

SEQ ID NO: 242 - protein, 24 aa, synthetic construct
GGYATQCWYFGGHVRGPCLEREGG

SEQ ID NO: 243 - protein, 24 aa, synthetic construct
GGKDNRCWYFAGTVLGPCLFGTGG

SEQ ID NO: 244 - protein, 24 aa, synthetic construct
GGHGELCWYFGGTVPGPCLDKVGG

SEQ ID NO: 245 - protein, 24 aa, synthetic construct
GGDEEQCWYFGGRVRGPCLLENGG

SEQ ID NO: 246 - protein, 24 aa, synthetic construct
GGVNNLCWYFGGTVLGPCLEGFGG

SEQ ID NO: 247 - protein, 24 aa, synthetic construct
GGDKRMCVYFGGTVLGPCLQDIGG

SEQ ID NO: 248 - protein, 20 aa, synthetic construct
EGFFCEYWGVTGLGPCPGIN

SEQ ID NO: 249 - protein, 20 aa, synthetic construct
IYGLCSYFGGTVWGLCLGDS

SEQ ID NO: 250 - protein, 20 aa, synthetic construct
GASQCWYFGGTVRGPCLGPI

SEQ ID NO: 251 - protein, 20 aa, synthetic construct
KGHGCWYLGGTGQGPCLGGK

SEQ ID NO: 252 - protein, 20 aa, synthetic construct
GDYLCWYFGGTVRGSCAGTT

SEQ ID NO: 253 - protein, 20 aa, synthetic construct
TSRLCWYFAGTVPGPCLEAI

SEQ ID NO: 254 - protein, 20 aa, synthetic construct
NNFMCWYFGGTVRAPCLQYM

SEQ ID NO: 255 - protein, 20 aa, synthetic construct
GSGICWYFAGTVPGPCLSQT

SEQ ID NO: 256 - protein, 20 aa, synthetic construct
GEKLCWYFGETVRGPCLKWM

SEQ ID NO: 257 - protein, 20 aa, synthetic construct
SSRLCWYFAGTVQGPCLSSL

SEQ ID NO: 258 - protein, 20 aa, synthetic construct
GEKLCWFFGGTVRAPCLNHM

SEQ ID NO: 259 - protein, 20 aa, synthetic construct
KDRRCWYFGGTVQGPCLSSN

SEQ ID NO: 260 - protein, 20 aa, synthetic construct
AAAPCWYFGGTVRGPCLGAR

SEQ ID NO: 261 - protein, 20 aa, synthetic construct
FSPLCYYFGGTVRGPCLGGT

SEQ ID NO: 262 - protein, 20 aa, synthetic construct
KGHLCSYFAGTVLGPCLWDT

SEQ ID NO: 263 - protein, 20 aa, synthetic construct
PVRLCWYFQETVRAPCLKFM

SEQ ID NO: 264 - protein, 20 aa, synthetic construct
GQYLCWYFGGAVSGTCLDDY

SEQ ID NO: 265 - protein, 20 aa, synthetic construct
SGVLCWYFGGRVPGPCLGQN

SEQ ID NO: 266 - protein, 20 aa, synthetic construct
REKLCVYFGGTVWGPCLDGT

SEQ ID NO: 267 - protein, 20 aa, synthetic construct
GKELCWYFAGTVRGPCLGSI

SEQ ID NO: 268 - protein, 20 aa, synthetic construct
WWGLCSYFGGTVSGPCLTER

SEQ ID NO: 269 - protein, 20 aa, synthetic construct
DSRLCWYFAGTVLGPCLHQI

SEQ ID NO: 270 - protein, 20 aa, synthetic construct
LTGLCSYFGGSVQGPCLAQS

SEQ ID NO: 271 - protein, 20 aa, synthetic construct
YATQCWYFGGHVRGPCLERE

SEQ ID NO: 272 - protein, 20 aa, synthetic construct
KDNRCWYFAGTVLGPCLFGT

SEQ ID NO: 273 - protein, 20 aa, synthetic construct
HGELCWYFGGTVPGPCLDKV

SEQ ID NO: 274 - protein, 20 aa, synthetic construct
DEEQCWYFGGRVRGPCLLEN

SEQ ID NO: 275 - protein, 20 aa, synthetic construct
VNNLCWYFGGTVLGPCLEGF

SEQ ID NO: 276 - protein, 20 aa, synthetic construct
DKRMCVYFGGTVLGPCLQDI

SEQ ID NO: 277 - protein, 18 aa, synthetic construct
GGICWYFGGQVSSKCLGG

SEQ ID NO: 278 - protein, 18 aa, synthetic construct
GGLCSYFAGTLLGPCLGG

SEQ ID NO: 279 - protein, 18 aa, synthetic construct
GGLCSFFQGTVRATCLGG

SEQ ID NO: 280 - protein, 18 aa, synthetic construct
GGSCLYFGGTVSGRCLGG

SEQ ID NO: 281 - protein, 18 aa, synthetic construct
GGSCVYFGGRVVGPCFGG

SEQ ID NO: 282 - protein, 18 aa, synthetic construct
GGVCWYFGHTVRSYCIGG

SEQ ID NO: 283 - protein, 18 aa, synthetic construct
GGWCRAFAGTVTYPCVGG

SEQ ID NO: 284 - protein, 18 aa, synthetic construct
GGYCFAGRGPWRAECMGG

SEQ ID NO: 285 - protein, 18 aa, synthetic construct
GGYCPADLQDYFMDCVGG

SEQ ID NO: 286 - protein, 14 aa, synthetic construct
ICWYFGGQVSSKCL

SEQ ID NO: 287 - protein, 14 aa, synthetic construct
LCSYFAGTLLGPCL

SEQ ID NO: 288 - protein, 14 aa, synthetic construct
LCSFFQGTVRATCL

SEQ ID NO: 289 - protein, 14 aa, synthetic construct
SCLYFGGTVSGRCL

SEQ ID NO: 290 - protein, 14 aa, synthetic construct
SCVYFGGRVVGPCF

SEQ ID NO: 291 - protein, 14 aa, synthetic construct
VCWYFGHTVRSYCI

SEQ ID NO: 292 - protein, 14 aa, synthetic construct
WCRAFAGTVTYPCV

SEQ ID NO: 293 - protein, 14 aa, synthetic construct
YCFAGRGPWRAECM

SEQ ID NO: 294 - protein, 14 aa, synthetic construct
YCPADLQDYFMDCV

SEQ ID NO: 295 - protein, 18 aa, synthetic construct
GGGCTPVFAGRGMWCVGG

SEQ ID NO: 296 - protein, 18 aa, synthetic construct
GGRCGRAFGGQVAFCFGG

SEQ ID NO: 297 - protein, 14 aa, synthetic construct
GCTPVFAGRGMWCV

SEQ ID NO: 298 - protein, 14 aa, synthetic construct
RCGRAFGGQVAFCF

SEQ ID NO: 299 - protein, 23 aa, synthetic construct
GGWKACPDWLSDHMSEIWCHQGG

SEQ ID NO: 300 - protein, 24 aa, synthetic construct
GGENVCPEWLSDHFSEMLCTRQGG

SEQ ID NO: 301 - protein, 24 aa, synthetic construct
GGFMDCPEWLSDHFYEMICHFRGG

SEQ ID NO: 302 - protein, 24 aa, synthetic construct
GGHLRCPKWLADHDSEMLCFRPGG

SEQ ID NO: 303 - protein, 24 aa, synthetic construct
GGSTGCPEWLSDHFYEMLCNFQGG

SEQ ID NO: 304 - protein, 24 aa, synthetic construct
GGTLGCPNWLSDHWGEILCSGFGG

SEQ ID NO: 305 - protein, 24 aa, synthetic construct
GGLWACPDWLADHRYEMLCYHSGG

SEQ ID NO: 306 - protein, 24 aa, synthetic construct
GGMNICPYWLSDHFSEMLCTSQGG

SEQ ID NO: 307 - protein, 24 aa, synthetic construct
GGASSCPDWLSDHFGEILCMSHGG

SEQ ID NO: 308 - protein, 24 aa, synthetic construct
GGQISCPNWLSDHFGEILCYRRGG

SEQ ID NO: 309 - protein, 24 aa, synthetic construct
GGLQVCPNWLSDHLGEFWCLGRGG

SEQ ID NO: 310 - protein, 24 aa, synthetic construct
GGQSRCPKWLSDHDGEMLCTRTGG

SEQ ID NO: 311 - protein, 24 aa, synthetic construct
GGRNMCPRWLSDHFGEMLCSDNGG

SEQ ID NO: 312 - protein, 24 aa, synthetic construct
GGSNKCPPWLSDHQYEVLCQVMGG

SEQ ID NO: 313 - protein, 24 aa, synthetic construct
GGMQNCPNWLSDHQGEMLCMVSGG

SEQ ID NO: 314 - protein, 24 aa, synthetic construct
GGNRLCPAWLSDHFGEQLCMALGG

SEQ ID NO: 315 - protein, 24 aa, synthetic construct
  VARIANT 3: note = Any amino acid
  VARIANT 20: note = Any amino acid
GGXKRCPKWLSDHRGEVVCXQNGG

SEQ ID NO: 316 - protein, 24 aa, synthetic construct
GGLNLCPEWLSDHWGEFLCFPRGG

SEQ ID NO: 317 - protein, 24 aa, synthetic construct
GGGNWCPKWLSDHFDEVICTGFGG

SEQ ID NO: 318 - protein, 24 aa, synthetic construct
GGRNMCPRWLSDHFGEILCFDNGG

SEQ ID NO: 319 - protein, 24 aa, synthetic construct
GGPYGCPNWLSDHFGELLCKKTGG

SEQ ID NO: 320 - protein, 19 aa, synthetic construct
WKACPDWLSDHMSEIWCHQ

SEQ ID NO: 321 - protein, 20 aa, synthetic construct
ENVCPEWLSDHFSEMLCTRQ

SEQ ID NO: 322 - protein, 20 aa, synthetic construct
FMDCPEWLSDHFYEMICHFR

SEQ ID NO: 323 - protein, 20 aa, synthetic construct
HLRCPKWLADHDSEMLCFRP

SEQ ID NO: 324 - protein, 20 aa, synthetic construct
STGCPEWLSDHFYEMLCNFQ

SEQ ID NO: 325 - protein, 20 aa, synthetic construct
TLGCPNWLSDHWGEILCSGF

SEQ ID NO: 326 - protein, 20 aa, synthetic construct
LWACPDWLADHRYEMLCYHS

SEQ ID NO: 327 - protein, 20 aa, synthetic construct
MNICPYWLSDHFSEMLCTSQ

SEQ ID NO: 328 - protein, 20 aa, synthetic construct
ASSCPDWLSDHFGEILCMSH

SEQ ID NO: 329 - protein, 20 aa, synthetic construct
QISCPNWLSDHFGEILCYRR

SEQ ID NO: 330 - protein, 20 aa, synthetic construct
LQVCPNWLSDHLGEFWCLGR

SEQ ID NO: 331 - protein, 20 aa, synthetic construct
QSRCPKWLSDHDGEMLCTRT

SEQ ID NO: 332 - protein, 20 aa, synthetic construct
RNMCPRWLSDHFGEMLCSDN

SEQ ID NO: 333 - protein, 20 aa, synthetic construct
SNKCPPWLSDHQYEVLCQVM

SEQ ID NO: 334 - protein, 20 aa, synthetic construct
MQNCPNWLSDHQGEMLCMVS

SEQ ID NO: 335 - protein, 20 aa, synthetic construct
NRLCPAWLSDHFGEQLCMAL

SEQ ID NO: 336 - protein, 20 aa, synthetic construct
  VARIANT 1: note = Any amino acid
  VARIANT 18: note = Any amino acid
XKRCPKWLSDHRGEVVCXQN

SEQ ID NO: 337 - protein, 20 aa, synthetic construct
LNLCPEWLSDHWGEFLCFPR

SEQ ID NO: 338 - protein, 20 aa, synthetic construct
GNWCPKWLSDHFDEVICTGF

SEQ ID NO: 339 - protein, 20 aa, synthetic construct
RNMCPRWLSDHFGEILCFDN

SEQ ID NO: 340 - protein, 20 aa, synthetic construct
PYGCPNWLSDHFGELLCKKT

SEQ ID NO: 341 - protein, 18 aa, synthetic construct
GGCPNWLSDHLSEIWCGG

SEQ ID NO: 342 - protein, 18 aa, synthetic construct
GGCPLWLGDHVGDLVCGG

SEQ ID NO: 343 - protein, 18 aa, synthetic construct
GGCPLWMSDHFYDMVCGG

SEQ ID NO: 344 - protein, 18 aa, synthetic construct
GGCPPWFFDHASEFICGG

SEQ ID NO: 345 - protein, 18 aa, synthetic construct
GGCPRWLDDHDGGYICGG

SEQ ID NO: 346 - protein, 18 aa, synthetic construct
GGCPDFLRDHRSEIICGG

SEQ ID NO: 347 - protein, 18 aa, synthetic construct
GGCGFLLTDHPSLHVCGG

SEQ ID NO: 348 - protein, 14 aa, synthetic construct
CPNWLSDHLSEIWC

SEQ ID NO: 349 - protein, 14 aa, synthetic construct
CPLWLGDHVGDLVC

SEQ ID NO: 350 - protein, 14 aa, synthetic construct
CPLWMSDHFYDMVC

SEQ ID NO: 351 - protein, 14 aa, synthetic construct
CPPWFFDHASEFIC

SEQ ID NO: 352 - protein, 14 aa, synthetic construct
CPRWLDDHDGGYIC

SEQ ID NO: 353 - protein, 14 aa, synthetic construct
CPDFLRDHRSEIIC

SEQ ID NO: 354 - protein, 14 aa, synthetic construct
CGFLLTDHPSLHVC

SEQ ID NO: 355 - protein, 20 aa, synthetic construct
GGRMCKYAFPDHPRLCLFGG

SEQ ID NO: 356 - protein, 16 aa, synthetic construct
RMCKYAFPDHPRLCLF

SEQ ID NO: 357 - protein, 18 aa, synthetic construct
GGMCPFQDHWREVWCWGG

SEQ ID NO: 358 - protein, 14 aa, synthetic construct
MCPFQDHWREVWCW

SEQ ID NO: 359 - protein, 18 aa, synthetic construct
GGWHCLNDHPELHCVRGG

SEQ ID NO: 360 - protein, 14 aa, synthetic construct
WHCLNDHPELHCVR

SEQ ID NO: 361 - protein, 19 aa, synthetic construct
GGILCQDHGWMIRECLLGG

SEQ ID NO: 362 - protein, 15 aa, synthetic construct
ILCQDHGWMIRECLL

SEQ ID NO: 363 - protein, 16 aa, synthetic construct
GGYALFGDGNWPWWGG

SEQ ID NO: 364 - protein, 18 aa, synthetic construct
GGLQWCFGGMVPCTLNGG

SEQ ID NO: 365 - protein, 19 aa, synthetic construct
GGFVACLQDHITACRWFGG

SEQ ID NO: 366 - protein, 19 aa, synthetic construct
GGNCFSAKSDWLFWMCEGG

SEQ ID NO: 367 - protein, 19 aa, synthetic construct
GGSCSWGYDWLHNEWCPGG

SEQ ID NO: 368 - protein, 19 aa, synthetic construct
GGYCQSVWMQQHFMSCHGG

SEQ ID NO: 369 - protein, 18 aa, synthetic construct
GGCPVWLLDHQDEFLCGG

SEQ ID NO: 370 - protein, 20 aa, synthetic construct
GGGSPCDDHPGWSPCYLVGG

SEQ ID NO: 371 - protein, 20 aa, synthetic construct
GGYSCYLMEPGDPYICYSGG

SEQ ID NO: 372 - protein, 20 aa, synthetic construct
GGQPCTAWRPGGKGFCRSGG

SEQ ID NO: 373 - protein, 20 aa, synthetic construct
GGVGCPSWVPGALGFCREGG

SEQ ID NO: 374 - protein, 18 aa, synthetic construct
GGGLCSWVGTDGVCYVGG

SEQ ID NO: 375 - protein, 20 aa, synthetic construct
GGNRMCSRYPGYYFCVQSGG

SEQ ID NO: 376 - protein, 18 aa, synthetic construct
GGWPCDYVDSGGTCIIGG

SEQ ID NO: 377 - protein, 17 aa, synthetic construct
GQCYRDYVIHDFVCQGG

SEQ ID NO: 378 - protein, 18 aa, synthetic construct
GGCVFPENSWVWDFYCGG

SEQ ID NO: 379 - protein, 18 aa, synthetic construct
GGTCSFLQQKIVDLCTGG

SEQ ID NO: 380 - protein, 19 aa, synthetic construct
GGSPCRFRSWYDDRCLVGG

SEQ ID NO: 381 - protein, 18 aa, synthetic construct
GGSCDFLQERILEICWGG

SEQ ID NO: 382 - protein, 12 aa, synthetic construct
YALFGDGNWPWW

SEQ ID NO: 383 - protein, 14 aa, synthetic construct
LQWCFGGMVPCTLN

SEQ ID NO: 384 - protein, 15 aa, synthetic construct
FVACLQDHITACRWF

SEQ ID NO: 385 - protein, 15 aa, synthetic construct
NCFSAKSDWLFWMCE

SEQ ID NO: 386 - protein, 15 aa, synthetic construct
SCSWGYDWLHNEWCP

SEQ ID NO: 387 - protein, 15 aa, synthetic construct
YCQSVWMQQHFMSCH

SEQ ID NO: 388 - protein, 14 aa, synthetic construct
CPVWLLDHQDEFLC

SEQ ID NO: 389 - protein, 16 aa, synthetic construct
GSPCDDHPGWSPCYLV

SEQ ID NO: 390 - protein, 16 aa, synthetic construct
YSCYLMEPGDPYICYS

SEQ ID NO: 391 - protein, 16 aa, synthetic construct
QPCTAWRPGGKGFCRS

SEQ ID NO: 392 - protein, 16 aa, synthetic construct
VGCPSWVPGALGFCRE

SEQ ID NO: 393 - protein, 14 aa, synthetic construct
GLCSWVGTDGVCYV

SEQ ID NO: 394 - protein, 16 aa, synthetic construct
NRMCSRYPGYYFCVQS

SEQ ID NO: 395 - protein, 14 aa, synthetic construct
WPCDYVDSGGTCII

SEQ ID NO: 396 - protein, 14 aa, synthetic construct
QCYRDYVIHDFVCQ

SEQ ID NO: 397 - protein, 14 aa, synthetic construct
CVFPENSWVWDFYC

SEQ ID NO: 398 - protein, 14 aa, synthetic construct
TCSFLQQKIVDLCT

SEQ ID NO: 399 - protein, 15 aa, synthetic construct
SPCRFRSWYDDRCLV

SEQ ID NO: 400 - protein, 14 aa, synthetic construct
SCDFLQERILEICW

SEQ ID NO: 401 - protein, 16 aa, synthetic construct
PAPAPAPAPAPAPAPA

SEQ ID NO: 402 - protein, 20 aa, synthetic construct
  VARIANT 1..20: note = This sequence may encompass 0-10 GS repeating units
GSGSGSGSGSGSGSGSGSGS

SEQ ID NO: 403 - protein, 24 aa, synthetic construct
  SITE 24: note = Amidated residue
GGREDMECFWSPGQVWCIPSYLGG

SEQ ID NO: 404 - protein, 18 aa, synthetic construct
  SITE 18: note = Amidated residue
GGLCWYFGGTVRGPCLGG

SEQ ID NO: 405 - protein, 20 aa, synthetic construct
  SITE 20: note = Amidated residue
REDMECFWSPGQVWCIPSYL

SEQ ID NO: 406 - protein, 14 aa, synthetic construct
  SITE 14: note = Amidated residue
LCWYFGGTVRGPCL

SEQ ID NO: 407 - protein, 24 aa, synthetic construct
  SITE 24: note = Amidated residue
GGTSRLCWYFAGTVPGPCLEAIGG

SEQ ID NO: 408 - protein, 20 aa, synthetic construct
  SITE 20: note = Amidated residue
TSRLCWYFAGTVPGPCLEAI

SEQ ID NO: 409 - protein, 22 aa, synthetic construct
GGTSRLCWYFAGTVPGPCLEAI

SEQ ID NO: 410 - protein, 26 aa, synthetic construct
  SITE 26: note = Amidated residue
GGREDMECFWSPGQVWCIPSYLGGSG

SEQ ID NO: 411 - protein, 22 aa, synthetic construct
  SITE 22: note = Amidated residue
TSRLCWYFAGTVPGPCLEAIGG

SEQ ID NO: 412 - protein, 26 aa, synthetic construct
GGREDMECFWSPGQVWCIPSYLGGSG

SEQ ID NO: 413 - protein, 189 aa, Homo sapiens
MAAEPVEDNCINFVAMKFIDNTLYFIENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 414 - protein, 194 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 415 - protein, 17 aa, synthetic construct
ICWVPFHYLDELMCTGG

SEQ ID NO: 416 - protein, 16 aa, synthetic construct
GGCPNWLSDHLSEIWC

SEQ ID NO: 417 - protein, 18 aa, synthetic construct
FFECFYSPGHEWCIPSGG

SEQ ID NO: 418 - protein, 16 aa, synthetic construct
GGLCWYFGGTVRGPCL

SEQ ID NO: 419 - protein, 23 aa, synthetic construct
  SITE 23: note = Amidated residue
REDMECFWSPGQVWCIPSYLGGS

SEQ ID NO: 420 - protein, 23 aa, synthetic construct
REDMECFWSPGQVWCIPSYLGGS

SEQ ID NO: 421 - protein, 24 aa, synthetic construct
GGLSPICWVPFHYVDYWMCNLEGG

SEQ ID NO: 422 - protein, 24 aa, synthetic construct
GGLSSICWVPFHYVDEAACQLGGG

SEQ ID NO: 423 - protein, 24 aa, synthetic construct
GGYLDTCWVPFHYVNYLMCNVEGG

SEQ ID NO: 424 - protein, 24 aa, synthetic construct
GGDSEVCWVPFHYINHLMCTHDGG

SEQ ID NO: 425 - protein, 24 aa, synthetic construct
GGNANICWVPFHYVNETMCNVGGG

SEQ ID NO: 426 - protein, 24 aa, synthetic construct
GGNETLCWVPFHYVDYLFCNHDGG

SEQ ID NO: 427 - protein, 24 aa, synthetic construct
GGLESPCWVPFHFLDERMCMSGGG

SEQ ID NO: 428 - protein, 24 aa, synthetic construct
GGDSSMCWVPFHYVDEFMCNEDGG

SEQ ID NO: 429 - protein, 24 aa, synthetic construct
GGSKQICWVPFHYVDEQFCNDRGG

SEQ ID NO: 430 - protein, 24 aa, synthetic construct
GGYSSICWVPFHYVDERACTGQGG

SEQ ID NO: 431 - protein, 24 aa, synthetic construct
GGENMNCWVPFHYIDYLLCEDVGG

SEQ ID NO: 432 - protein, 21 aa, synthetic construct
GGICWVPFHYVDELMCNLEGG

SEQ ID NO: 433 - protein, 22 aa, synthetic construct
GGLICWVPWEDVDDRICTEGGG

SEQ ID NO: 434 - protein, 24 aa, synthetic construct
GGESFLECFYSPSAEWCIPSVIGG

SEQ ID NO: 435 - protein, 14 aa, synthetic construct
RSDGFVWWLGWLGG

SEQ ID NO: 436 - protein, 14 aa, synthetic construct
MFEAEWFLEQFGGG

SEQ ID NO: 437 - protein, 24 aa, synthetic construct
  SITE 1: note = Acetylated residue
  SITE 24: note = Amidated residue
GGREDMECFWSPGQVWCIPSYLGG

SEQ ID NO: 438 - protein, 22 aa, synthetic construct
  SITE 1: note = Acetylated residue
  SITE 22: note = Amidated residue
REDMECFWSPGQVWCIPSYLGG

SEQ ID NO: 439 - protein, 21 aa, synthetic construct
  SITE 1: note = Acetylated residue
  SITE 21: note = Amidated residue
EDMECFWSPGQVWCIPSYLGG

SEQ ID NO: 440 - protein, 20 aa, synthetic construct
  SITE 1: note = Acetylated residue
  SITE 20: note = Amidated residue
DMECFWSPGQVWCIPSYLGG

SEQ ID NO: 441 - protein, 19 aa, synthetic construct
  SITE 1: note = Acetylated residue
  SITE 19: note = Amidated residue
MECFWSPGQVWCIPSYLGG

SEQ ID NO: 442 - protein, 18 aa, synthetic construct
  SITE 1: note = Acetylated residue
  SITE 18: note = Amidated residue
ECFWSPGQVWCIPSYLGG

SEQ ID NO: 443 - protein, 22 aa, synthetic construct
  SITE 1: note = Acetylated residue
  SITE 22: note = Amidated residue
GGREDMECFWSPGQVWCIPSYL

SEQ ID NO: 444 - protein, 21 aa, synthetic construct
  SITE 1: note = Acetylated residue
  SITE 21: note = Amidated residue
GGREDMECFWSPGQVWCIPSY

SEQ ID NO: 445 - protein, 20 aa, synthetic construct
  SITE 1: note = Acetylated residue
  SITE 20: note = Amidated residue
GGREDMECFWSPGQVWCIPS

SEQ ID NO: 446 - protein, 18 aa, synthetic construct
GGYVCLWDAQVALCLVGG

SEQ ID NO: 447 - protein, 18 aa, synthetic construct
GGFCSFGDGTLPACLIGG

SEQ ID NO: 448 - protein, 24 aa, synthetic construct
GGFMDCPEWLSDHFYEMLCHFRGG

SEQ ID NO: 449 - protein, 18 aa, synthetic construct
GGQCYRDYVIHDFVCQGG

SEQ ID NO: 450 - protein, 26 aa, synthetic construct
  SITE 26: note = Amidated residue
GSGGREDMECFWSPGQVWCIPSYLGG

SEQ ID NO: 451 - protein, 16 aa, synthetic construct
  SITE 16: note = Amidated residue
LCWYFGGTVRGPCLGG

SEQ ID NO: 452 - protein, 22 aa, synthetic construct
  SITE 22: note = Amidated residue
GSGGFFECFYSPGHEWCIPSGG

SEQ ID NO: 453 - protein, 16 aa, synthetic construct
  SITE 16: note = Amidated residue
FEWTPGYWQPYALPLL

SEQ ID NO: 454 - protein, 20 aa, synthetic construct
  VARIANT 1: note = Residue may be absent
  VARIANT 2: note = Residue may be absent
  VARIANT 3: note = Any amino acid or absent
  VARIANT 4: note = S, Q, E, G, L, K, T, A, or I
  VARIANT 7: note = I, Q, L, V, E, R, A, or K
  VARIANT 9: note = F or Y
  VARIANT 10: note = E, Q, H, T, R, S, G, M, or N
  VARIANT 11: note = H, W, Y, or F
  VARIANT 12: note = I, L, M, V, F, Y, W, A, G, P, S, or T
  VARIANT 13: note = D, A, R, N, S, K, T, or Q
  VARIANT 14: note = C or E
  VARIANT 15: note = G, P, D, Q, E, L, V, or H
  VARIANT 16: note = G, P, D, I, S, E, Q, K, I, or M
  VARIANT 17: note = G, C, or absent
  VARIANT 18: note = G, T, or absent
  VARIANT 19: note = Residue may be absent
  VARIANT 20: note = Residue may be absent
GGXXCWXPXXXXXXXXXXGG

SEQ ID NO: 455 - protein, 20 aa, synthetic construct
  VARIANT 1: note = Residue may be absent
  VARIANT 2: note = Residue may be absent
  VARIANT 3: note = Any amino acid
  VARIANT 4: note = L or I
  VARIANT 7: note = R or K
  VARIANT 10: note = S or T
  VARIANT 11: note = F, H, W, or Y
  VARIANT 12: note = I, L, M, V, F, Y, W, A, G, P, S, or T
  VARIANT 15: note = E or D
  VARIANT 16: note = E, D, or Q
  VARIANT 17: note = G, C, or absent
  VARIANT 18: note = G, T, or absent
  VARIANT 19: note = Residue may be absent
  VARIANT 20: note = Residue may be absent
GGXXCWXPFXXXDCXXXXGG

SEQ ID NO: 456 - protein, 20 aa, synthetic construct
  VARIANT 19: note = Residue may be absent
  VARIANT 20: note = Residue may be absent
GGELCWRPFSFVDCEEGGGG

SEQ ID NO: 457 - protein, 20 aa, synthetic construct
  VARIANT 1: note = Residue may be absent
  VARIANT 2: note = Residue may be absent
  VARIANT 3: note = F or S
  VARIANT 4: note = N or W
  VARIANT 5: note = V, A, or L
  VARIANT 7: note = D or S
  VARIANT 8: note = F or L
  VARIANT 9: note = D or E
  VARIANT 14: note = D, G, or E
  VARIANT 16: note = Any amino acid
  VARIANT 17: note = Any amino acid
  VARIANT 18: note = Any amino acid
  VARIANT 19: note = Residue may be absent
  VARIANT 20: note = Residue may be absent
GGXXXCXXXPGWWXCXXXGG

SEQ ID NO: 458 - protein, 20 aa, synthetic construct
  VARIANT 1: note = Residue may be absent
  VARIANT 2: note = Residue may be absent
  VARIANT 5: note = I, L, M, V, F, Y, W, A, G, P, S, or T
  VARIANT 8: note = I, L, M, V, F, Y, W, A, G, P, S, or T
  VARIANT 9: note = D or E
  VARIANT 14: note = D or E
  VARIANT 16: note = Any amino acid
  VARIANT 17: note = Any amino acid
  VARIANT 18: note = Any amino acid
  VARIANT 19: note = Residue may be absent
  VARIANT 20: note = Residue may be absent
GGFNXCSXXPGWWXCXXXGG

SEQ ID NO: 459 - protein, 20 aa, synthetic construct
  VARIANT 1: note = Residue may be absent
  VARIANT 2: note = Residue may be absent
  VARIANT 3: note = Any amino acid
  VARIANT 5: note = Any amino acid
  VARIANT 7: note = E or D
  VARIANT 9: note = I, L, M, V, F, Y, W, A, G, P, S, or T
  VARIANT 12: note = Any amino acid
  VARIANT 13: note = F, H, W, or Y
  VARIANT 14: note = I, L, M, V, F, Y, W, A, G, P, S, or T
  VARIANT 16: note = Any amino acid
  VARIANT 17: note = E, C, or D
  VARIANT 18: note = Any amino acid
  VARIANT 19: note = Residue may be absent
  VARIANT 20: note = Residue may be absent
GGXCXLXWXPFXXXEXXXGG

SEQ ID NO: 460 - protein, 24 aa, synthetic construct
  VARIANT 1: note = Residue may be absent
  VARIANT 2: note = Residue may be absent
  VARIANT 3: note = Any amino acid
  VARIANT 4: note = N, D, or E
  VARIANT 5: note = I, L, M, V, F, Y, W, A, G, P, S, T, H, or Y
  VARIANT 6: note = I, L, M, V, F, Y, W, A, G, P, S, or T
  VARIANT 7: note = E, or D
  VARIANT 10: note = W or Y
  VARIANT 13: note = G or S
  VARIANT 21: note = I, L, M, V, F, Y, W, A, G, P, S, or T
  VARIANT 22: note = I, L, M, V, F, Y, W, A, G, P, S, or T
  VARIANT 23: note = Residue may be absent
  VARIANT 24: note = Residue may be absent
GGXXXXXCFXSPXQEWCIPSXXGG

SEQ ID NO: 461 - protein, 20 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 6: note = E, Y, or W
  VARIANT 7: note = S, G, P, or T
  VARIANT 8: note = M, D, or Q
  VARIANT 9: note = E, S, N, or P
  VARIANT 12: note = Q, D, or I
  VARIANT 13: note = Y or D
  VARIANT 14: note = T, Y, W, or K
  VARIANT 15: note = E, V, or N
  VARIANT 17: note = Any amino acid
  VARIANT 18: note = F, H, W, E, P, V, or Y
  VARIANT 19: note = Residue may be absent
  VARIANT 20: note = Residue may be absent
GGXXCXXXXPGXXXXCXXGG

SEQ ID NO: 462 - protein, 20 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 6: note = Y or W
  VARIANT 8: note = D or Q
  VARIANT 9: note = E or N
  VARIANT 14: note = Y or W
  VARIANT 15: note = E or N
  VARIANT 17: note = Any amino acid
  VARIANT 18: note = F, H, W, or Y
  VARIANT 19: note = Residue may be absent
  VARIANT 20: note = Residue may be absent
GGXXCXPXXPGIYXXCXXGG

SEQ ID NO: 463 - protein, 24 aa, synthetic construct
  VARIANT 1: note = Residue may be absent
  VARIANT 2: note = Residue may be absent
  VARIANT 3: note = G, Q, or absent
  VARIANT 4: note = Any amino acid
  VARIANT 5: note = Any amino acid
  VARIANT 10: note = E, N, or D
  VARIANT 11: note = S or P
  VARIANT 13: note = Any amino acid
  VARIANT 14: note = Any amino acid
  VARIANT 15: note = I, L, M, V, F, Y, W, A, G, P, S, or T
  VARIANT 16: note = I, L, M, V, F, Y, W, A, G, P, S, T, or H
  VARIANT 18: note = Any amino acid
  VARIANT 19: note = Any amino acid
  VARIANT 20: note = Any amino acid
  VARIANT 21: note = Any amino acid
  VARIANT 22: note = Residue may be absent
  VARIANT 23: note = Residue may be absent
  VARIANT 24: note = Residue may be absent
GGXXXCWQDXXGXXXXCXXXXSGG

SEQ ID NO: 464 - protein, 20 aa, synthetic construct
  VARIANT 1: note = Residue may be absent
  VARIANT 2: note = Residue may be absent
  VARIANT 3: note = Any amino acid
  VARIANT 4: note = D or N
  VARIANT 6: note = I, L, M, V, F, Y, W, A, G, P, S, or T
  VARIANT 12: note = D or E
  VARIANT 15: note = P or W
  VARIANT 17: note = Any amino acid
  VARIANT 18: note = Any amino acid
  VARIANT 19: note = Residue may be absent
  VARIANT 20: note = Residue may be absent
GGXXCXPLWPGXADXCXXGG

SEQ ID NO: 465 - protein, 20 aa, synthetic construct
  VARIANT 1: note = Residue may be absent
  VARIANT 20: note = Residue may be absent
GGGYVCLWDAQVALCLVGGG

SEQ ID NO: 466 - protein, 20 aa, synthetic construct
  VARIANT 1: note = Residue may be absent
  VARIANT 20: note = Residue may be absent
GGGFCSFGDGTLPACLIGGG

SEQ ID NO: 467 - protein, 24 aa, synthetic construct
  VARIANT 1: note = Residue may be absent
  VARIANT 2: note = Residue may be absent
  VARIANT 3: note = Any amino acid or absent
  VARIANT 4: note = Any amino acid or absent
  VARIANT 5: note = Any amino acid or absent
  VARIANT 6: note = I, L, M, V, F, Y, W, A, G, P, S, or T
  VARIANT 8: note = I, L, M, V, F, Y, W, A, G, P, S, or T
  VARIANT 9: note = Y or F
  VARIANT 16: note = Any amino acid
  VARIANT 17: note = Any amino acid
  VARIANT 20: note = Any amino acid or absent
  VARIANT 21: note = Any amino acid or absent
  VARIANT 22: note = W, A, G, P, N, S, I, K, T, M, L, N, R, Y, E, V, F, or absent
  VARIANT 23: note = Residue may be absent
  VARIANT 24: note = Residue may be absent
GGXXXXCXXFGGTVRXXCLXXXGG

SEQ ID NO: 468 - protein, 24 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 5: note = Any amino acid
  VARIANT 20: note = Any amino acid
  VARIANT 21: note = Any amino acid
  VARIANT 22: note = I, L, M, V, F, Y, W, A, G, P, S, or T
  VARIANT 23: note = Residue may be absent
  VARIANT 24: note = Residue may be absent
GGXXXLCWYFGGTVRGPCLXXXGG

SEQ ID NO: 469 - protein, 24 aa, synthetic construct
  VARIANT 1: note = Residue may be absent
  VARIANT 2: note = Residue may be absent
  VARIANT 3: note = Any amino acid or absent
  VARIANT 4: note = Any amino acid or absent
  VARIANT 5: note = Any amino acid or absent
  VARIANT 6: note = M, A, T C, I, L, S, V, W, Y, F, Q, G, M, R, or P
  VARIANT 7: note = T, G, or C
  VARIANT 8: note = I, L, M, V, W, A, G, T, P, R, W, S, L, V, F, E, or Y
  VARIANT 10: note = F, G, D, W, or L
  VARIANT 11: note = A, G, Q, R, or L
  VARIANT 12: note = G, H, Q, V, or E
  VARIANT 13: note = R, Q, T, P, D, A, S, or H
  VARIANT 14: note = I, L, M, V, F, Y, W, A, G, P, S, or T
  VARIANT 15: note = I, L, M, V, F, Y, W, A, G, P, S, or T
  VARIANT 16: note = Any amino acid
  VARIANT 17: note = Any amino acid
  VARIANT 18: note = V, F, or C
  VARIANT 19: note = I, L, M, V, F, Y, W, A, G, P, S, or T
  VARIANT 20: note = Any amino acid or absent
  VARIANT 21: note = Any amino acid or absent
  VARIANT 22: note = W, A, G, P, N, S, I, K, T, M, L, N, R, Y, E, V, F, or absent
  VARIANT 23: note = Residue may be absent
  VARIANT 24: note = Residue may be absent
GGXXXXXXAXXXXXXXXXXXXXGG

SEQ ID NO: 470 - protein, 24 aa, synthetic construct
  VARIANT 1: note = Residue may be absent
  VARIANT 2: note = Residue may be absent
  VARIANT 3: note = Any amino acid or absent
  VARIANT 4: note = R, G, N, M, L, T, W, S, I, Q, K, Y, or absent
  VARIANT 5: note = M, G, V, D, R, A, I, S, K, N, L, W, or absent
  VARIANT 6: note = Any amino acid
  VARIANT 8: note = D, E, or N
  VARIANT 10: note = I, L, M, V, F, Y, W, A, G, P, S, or T
  VARIANT 15: note = G or S
  VARIANT 16: note = D or E
  VARIANT 17: note = I, L, M, V, F, Y, W, A, G, P, S, or T
  VARIANT 18: note = I, L, M, V, F, Y, W, A, G, P, S, or T
  VARIANT 19: note = F or C
  VARIANT 20: note = Any amino acid
  VARIANT 21: note = G, R, F, H, S, D, V, A, Q, P, K, or absent
  VARIANT 22: note = G, Q, R, P, F, S, H, T, M, N, L, or absent
  VARIANT 23: note = Residue may be absent
  VARIANT 24: note = Residue may be absent
GGXXXXPXWXSDHFXXXXXXXXGG

SEQ ID NO: 471 - protein, 20 aa, synthetic construct
  VARIANT 1: note = Residue may be absent
  VARIANT 2: note = Residue may be absent
  VARIANT 3: note = F, H, W, or Y
  VARIANT 4: note = Any amino acid
  VARIANT 6: note = I, L, M, V, F, Y, W, A, G, P, S, or T
  VARIANT 12: note = I, L, M, V, F, Y, W, A, G, P, S, or T
  VARIANT 13: note = I, L, M, V, F, Y, W, A, G, P, S, or T
  VARIANT 15: note = I, L, M, V, F, Y, W, A, G, P, S, or T
  VARIANT 16: note = Any amino acid
  VARIANT 17: note = Any amino acid
  VARIANT 18: note = Any amino acid
  VARIANT 19: note = Residue may be absent
  VARIANT 20: note = Residue may be absent
GGXXCXNDHPEXXCXXXXGG

SEQ ID NO: 472 - protein, 24 aa, synthetic construct
  VARIANT 1: note = Residue may be absent
  VARIANT 2: note = Residue may be absent
  VARIANT 3: note = L, Y, D, N, S, or E
  VARIANT 4: note = S, L, A, E, K, N, or G
  VARIANT 5: note = P, S, D, E, N, T, Q, M, or G
  VARIANT 6: note = I, T, V, L, P, M, N, F, Y, W, A, G, or S
  VARIANT 13: note = Y, or F
  VARIANT 14: note = I, L, M, V, F, Y, W, A, G, P, S, or T
  VARIANT 15: note = D, or N
  VARIANT 16: note = Y, E, or H
  VARIANT 17: note = Any amino acid
  VARIANT 18: note = M, A, F, L, I, V, Y, W, G, P, S, T
  VARIANT 20: note = N, Q, T, M, or E
  VARIANT 21: note = Any amino acid
  VARIANT 22: note = E, G, D, R, Q, or V
  VARIANT 23: note = Residue may be absent
  VARIANT 24: note = Residue may be absent
GGXXXXCWVPFHXXXXXXCXXXGG

SEQ ID NO: 473 - protein, 24 aa, synthetic construct
  VARIANT 1: note = Residue may be absent
  VARIANT 2: note = Residue may be absent
  VARIANT 3: note = L, Y, D, N, S, or E
  VARIANT 4: note = S, L, A, E, K, N, or G
  VARIANT 5: note = P, S, D, E, N, T, Q, M, or G
  VARIANT 6: note = I, L, M, V, F, Y, W, A, G, P, S, or T
  VARIANT 17: note = Any amino acid
  VARIANT 21: note = Any amino acid
  VARIANT 22: note = D or E
  VARIANT 23: note = Residue may be absent
  VARIANT 24: note = Residue may be absent
GGXXXXCWVPFHYVDEXMCNXXGG

SEQ ID NO: 474 - protein, 24 aa, synthetic construct
  VARIANT 1: note = Residue may be absent
  VARIANT 2: note = Residue may be absent
  VARIANT 3: note = L, Y, D, N, S, or E
  VARIANT 4: note = S, L, A, E, K, N, or G
  VARIANT 5: note = P, S, D, E, N, T, Q, M, or G
  VARIANT 6: note = I, T, V, L, P, M, N, F, Y, W, A, G, or S
  VARIANT 13: note = Y, or F
  VARIANT 14: note = I, L, M, V, F, Y, W, A, G, P, S, or T
  VARIANT 17: note = Any amino acid
  VARIANT 18: note = I, L, M, V, F, Y, W, A, G, P, S, or T
  VARIANT 20: note = N, Q, T, M, or E
  VARIANT 21: note = Any amino acid
  VARIANT 22: note = E, G, D, R, Q, or V
  VARIANT 23: note = Residue may be absent
  VARIANT 24: note = Residue may be absent
GGXXXXCWVPFHXXNYXXCXXXGG

SEQ ID NO: 475 - protein, 24 aa, synthetic construct
  VARIANT 1: note = Residue may be absent
  VARIANT 2: note = Residue may be absent
  VARIANT 3: note = L, Y, D, N, S, or E
GGXGGICWVPFHYVDELMCNLEGG

SEQ ID NO: 476 - protein, 24 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 5: note = L, M, R, S, T, Y, Q, W, I, V, F, A, G, or P
  VARIANT 6: note = L, M, I, T, S, V, F, Y, W, A, G, or P
  VARIANT 8: note = F, S, or W
  VARIANT 9: note = R, V, L, M, H, W, F, I, Y, A, G, P, S, or T
  VARIANT 11: note = W, or F
  VARIANT 12: note = E, P, Q, N, or S
  VARIANT 13: note = D, G, or E
  VARIANT 14: note = V, L, I, T, F, M, Y, W, A, G, P, or S
  VARIANT 15: note = E, A, D, G, F, or M
  VARIANT 17: note = L, F, H, R, or S
  VARIANT 18: note = V, I, F, L, M, Y, W, A, G, P, S, or T
  VARIANT 20: note = T, M, I, W, or S
  VARIANT 21: note = E, G, S, T, A, W, D, or Y
  VARIANT 22: note = Any amino acid
  VARIANT 23: note = Residue may be absent
  VARIANT 24: note = Residue may be absent
GGXXXXCXXPXXXXXDXXCXXXGG

SEQ ID NO: 477 - protein, 24 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 6: note = I, L, M, V, F, Y, W, A, G, P, S, or T
  VARIANT 15: note = D or E
  VARIANT 22: note = Any amino acid
  VARIANT 23: note = Residue may be absent
  VARIANT 24: note = Residue may be absent
GGXXLXCWVPWEDVXDRICTEXGG

SEQ ID NO: 478 - protein, 24 aa, synthetic construct
  VARIANT 1: note = Residue may be absent
  VARIANT 2: note = Residue may be absent
  VARIANT 3: note = Any amino acid
  VARIANT 4: note = Any amino acid
  VARIANT 5: note = I, L, M, V, F, Y, W, A, G, P, S, or T
  VARIANT 6: note = L, M, I, T, S, V, F, Y, W, A, G, or P
  VARIANT 9: note = I, L, M, V, F, Y, W, A, G, P, S, or T
  VARIANT 11: note = W, or F
  VARIANT 12: note = Q or N
  VARIANT 13: note = D, G, or E
  VARIANT 14: note = I, L, M, V, F, Y, W, A, G, P, S, or T
  VARIANT 15: note = E, A, D, G, F, or M
  VARIANT 18: note = I, L, M, V, F, Y, W, A, G, P, S, or T
  VARIANT 20: note = T, M, I, W, or S
  VARIANT 21: note = E, G, S, T, A, W, D, or Y
  VARIANT 22: note = Any amino acid
  VARIANT 23: note = Residue may be absent
  VARIANT 24: note = Residue may be absent
GGXXXXCFXPXXXXXDHXCXXXGG

SEQ ID NO: 479 - protein, 24 aa, synthetic construct
  VARIANT 1: note = Residue may be absent
  VARIANT 2: note = Residue may be absent
GGGGLICWVPWEDVDDRICTEGGG

SEQ ID NO: 480 - protein, 22 aa, synthetic construct
YLDTCWVPFHYVNYLMCNVEGG

SEQ ID NO: 481 - protein, 22 aa, synthetic construct
  SITE 22: note = Amidated residue
YLDTCWVPFHYVNYLMCNVEGG

SEQ ID NO: 482 - protein, 22 aa, synthetic construct
  SITE 22: note = Amidated residue
REDMECFWSPGQVWCIPSYLGG

SEQ ID NO: 483 - protein, 16 aa, synthetic construct
FEWTPGYWQPYALPLL

SEQ ID NO: 484 - protein, 20 aa, synthetic construct
  VARIANT 1: note = Residue may be absent
  VARIANT 2: note = Residue may be absent
  VARIANT 3: note = Any amino acid or absent
  VARIANT 4: note = L, C, or G
  VARIANT 5: note = Any amino acid
  VARIANT 6: note = C, L, or S
  VARIANT 7: note = D, V, E, or R
  VARIANT 8: note = Y or W
  VARIANT 9: note = I, L, M, V, F, Y, W, A, G, P, S, or T
  VARIANT 11: note = G or F
  VARIANT 12: note = Any amino acid
  VARIANT 13: note = W, H, S, F, or Y
  VARIANT 14: note = I, L, M, V, F, Y, W, A, G, P, S, or T
  VARIANT 15: note = C, E, or V
  VARIANT 16: note = Any amino acid
  VARIANT 17: note = E, C, or D
  VARIANT 18: note = Any amino acid or absent
  VARIANT 19: note = Residue may be absent
  VARIANT 20: note = Residue may be absent
GGXXXXXXXPXXXXXXXXGG

SEQ ID NO: 485 - protein, 24 aa, synthetic construct
  VARIANT 1: note = Residue may be absent
  VARIANT 2: note = Residue may be absent
  VARIANT 3: note = Any amino acid or absent
  VARIANT 4: note = G, N, S, V, D, Y, E, A, or absent
  VARIANT 5: note = M, A, P, T, W, F, G, Y, F, S, V, H, D, K, L, I, or absent
  VARIANT 6: note = I, V, Y, A, G, P, T, S, F, M, L, or W
  VARIANT 7: note = Q, E, or D
  VARIANT 9: note = L, F, W, A, or Y
  VARIANT 10: note = W or Y
  VARIANT 11: note = E or S
  VARIANT 13: note = L, G, H, T, S, Y, or T
  VARIANT 14: note = W, H, E, Q, or A
  VARIANT 15: note = E, V, Y, H, or L
  VARIANT 16: note = C or W
  VARIANT 17: note = W or C
  VARIANT 18: note = T, I, S, M, or W
  VARIANT 19: note = D, P, or T
  VARIANT 20: note = G, S, H, I, T, A, or absent
  VARIANT 21: note = M, W, A, P, S, T, G, L, V, F, Y, Q, I, or absent
  VARIANT 22: note = V, F, Y, A, S, G, M, I, T, N, F, D, L, Q, W, P, or absent
  VARIANT 23: note = Residue may be absent
  VARIANT 24: note = Residue may be absent
GGXXXXXCXXXPXXXXXXXXXXGG

SEQ ID NO: 486 - protein, 24 aa, synthetic construct
  VARIANT 1: note = Residue may be absent
  VARIANT 2: note = Residue may be absent
  VARIANT 3: note = G, Q, or absent
  VARIANT 4: note = Any amino acid or absent
  VARIANT 5: note = Any amino acid
  VARIANT 6: note = L or C
  VARIANT 7: note = H, F, or W
  VARIANT 8: note = C, W, Q, or I
  VARIANT 9: note = F, V, D, or S
  VARIANT 10: note = E, N, Y, D, or L
  VARIANT 11: note = S, D, or P
  VARIANT 12: note = R, M, or G
  VARIANT 13: note = Any amino acid
  VARIANT 14: note = Any amino acid
  VARIANT 15: note = M, F, Y, W, A, G, P, S, T, D, V, L, or I
  VARIANT 16: note = I, L, M, F, Y, A, G, P, S, T, F, H, W, V, or Y
  VARIANT 18: note = Any amino acid
  VARIANT 19: note = Any amino acid or absent
  VARIANT 20: note = Any amino acid or absent
  VARIANT 21: note = Any amino acid or absent
  VARIANT 22: note = Residue may be absent
  VARIANT 23: note = Residue may be absent
  VARIANT 24: note = Residue may be absent
GGXXXXXXXXXXXXXXCXXXXSGG

SEQ ID NO: 487 - protein, 20 aa, synthetic construct
  VARIANT 1: note = Residue may be absent
  VARIANT 2: note = Residue may be absent
  VARIANT 3: note = Any amino acid
  VARIANT 4: note = N, C, F, D, or S
  VARIANT 5: note = D, R, L, or C
  VARIANT 6: note = I, M, F, W, A, G, P, Y, T, S, E, V, L, or W
  VARIANT 7: note = F or P
  VARIANT 8: note = N, L, or T
  VARIANT 9: note = R, S, P, or W
  VARIANT 10: note = L, R, or P
  VARIANT 11: note = W, L, V, or G
  VARIANT 12: note = S, W, D, or E
  VARIANT 13: note = L, F, M, N, T, E, or A
  VARIANT 14: note = P, I, F, T, S, I, or D
  VARIANT 15: note = D, H, C, P, S, or W
  VARIANT 16: note = C or W
  VARIANT 17: note = Any amino acid or absent
  VARIANT 18: note = Any amino acid or absent
  VARIANT 19: note = Residue may be absent
  VARIANT 20: note = Residue may be absent
GGXXXXXXXXXXXXXXXXGG

SEQ ID NO: 488 - protein, 20 aa, synthetic construct
  VARIANT 1: note = Residue may be absent
  VARIANT 2: note = Residue may be absent
  VARIANT 3: note = G, R, or absent
  VARIANT 4: note = Y, K, F, or P
  VARIANT 5: note = T, V, F, H, P, C, Y, I, L, M, W, A, G, or S
  VARIANT 6: note = S, C, or Q
  VARIANT 7: note = I, Y, W, G, P, T, F, M, D, A, H, V, L, or S
  VARIANT 8: note = H, N, Y, F, W, L, or G
  VARIANT 9: note = D, V, I, R, E, or G
  VARIANT 10: note = Any amino acid
  VARIANT 11: note = Any amino acid
  VARIANT 12: note = I, F, Y, A, G, P, E, N, S, W, M, V, T, or L
  VARIANT 13: note = Any amino acid
  VARIANT 14: note = M, Y, W, G, S, T, L, I, V, E, A, F, or P
  VARIANT 15: note = C, D, or Y
  VARIANT 16: note = V, F, Y, W, A, G, P, T, L, M, I, S, R, or C
  VARIANT 17: note = F, Y, W, A, G, P, S, T, V, I, L, M, or R
  VARIANT 18: note = G, R, L, or absent
  VARIANT 19: note = Residue may be absent
  VARIANT 20: note = Residue may be absent
GGXXXXXXXXXXXXXXXXGG

SEQ ID NO: 489 - protein, 20 aa, synthetic construct
  VARIANT 1: note = Residue may be absent
  VARIANT 2: note = Residue may be absent
  VARIANT 3: note = G, R, or absent
  VARIANT 4: note = F or Y
  VARIANT 5: note = I, L, M, V, F, Y, W, A, G, P, S, or T
  VARIANT 7: note = I, L, M, V, F, Y, W, A, G, P, S, or T
  VARIANT 8: note = F, H, W, or Y
  VARIANT 9: note = D or E
  VARIANT 10: note = Any amino acid
  VARIANT 11: note = Any amino acid
  VARIANT 12: note = I, L, M, V, F, Y, W, A, G, P, S, or T
  VARIANT 13: note = Any amino acid
  VARIANT 14: note = I, L, M, V, F, Y, W, A, G, P, S, or T
  VARIANT 16: note = I, L, M, V, F, Y, W, A, G, P, S, or T
  VARIANT 17: note = I, L, M, V, F, Y, W, A, G, P, S, or T
  VARIANT 18: note = G, R, L, or absent
  VARIANT 19: note = Residue may be absent
  VARIANT 20: note = Residue may be absent
GGXXXCXXXXXXXXCXXXGG

SEQ ID NO: 490 - protein, 24 aa, synthetic construct
  VARIANT 1: note = Residue may be absent
  VARIANT 2: note = Residue may be absent
  VARIANT 3: note = Any amino acid or absent
  VARIANT 4: note = Any amino acid or absent
  VARIANT 5: note = Any amino acid or absent
  VARIANT 6: note = M, A, T C, I, L, S, V, W, Y, F, Q, G, M, R, or P
  VARIANT 7: note = T, G, or C
  VARIANT 8: note = I, L, M, V, W, A, G, T, P, R, W, S, L, V, F, E, or Y
  VARIANT 9: note = I, L, M, W, G, P, S, T, V, A, Y, or F
  VARIANT 10: note = F, G, D, W, or L
  VARIANT 11: note = A, G, Q, R, or L
  VARIANT 12: note = G, H, Q, V, or E
  VARIANT 13: note = R, Q, T, P, D, A, S, or H
  VARIANT 14: note = G, V, L, W, or Y
  VARIANT 15: note = I, Y, G, M, A, S, L, R, V, T, F, W, Q, or P
  VARIANT 16: note = Any amino acid
  VARIANT 17: note = Any amino acid
  VARIANT 18: note = V, F, or C
  VARIANT 19: note = Y, W, S, T, G, L, F, I, V, M, P, A, or absent
  VARIANT 20: note = Any amino acid or absent
  VARIANT 21: note = Any amino acid or absent
  VARIANT 22: note = W, A, G, P, N, S, I, K, T, M, L, N, R, Y, E, V, F, or absent
  VARIANT 23: note = Residue may be absent
  VARIANT 24: note = Residue may be absent
GGXXXXXXXXXXXXXXXXXXXXGG

SEQ ID NO: 491 - protein, 24 aa, synthetic construct
  VARIANT 1: note = Residue may be absent
  VARIANT 2: note = Residue may be absent
  VARIANT 3: note = Any amino acid or absent
  VARIANT 4: note = R, G, N, M, L, T, W, S, I, Q, K, Y, or absent
  VARIANT 5: note = M, G, V, D, R, A, I, S, K, N, L, W, or absent
  VARIANT 6: note = Any amino acid or absent
  VARIANT 7: note = K, M, P, or G
  VARIANT 8: note = Y, C, N, L, P, R, D, F, E, K, or A
  VARIANT 9: note = A, P, W, F, or L
  VARIANT 10: note = F, L, M, I, V, Y, W, A, G, P, S, or T
  VARIANT 11: note = P, Q, S, G, F, D, R, T, or A
  VARIANT 14: note = P, W, L, V, F, A, D, R, Q, or M
  VARIANT 15: note = R, S, G, Y, or D
  VARIANT 16: note = L, E, D, or G
  VARIANT 17: note = W, A, G, P, S, T, C, V, I, L, M, F, Y, H, or Q
  VARIANT 18: note = M, F, Y, A, G, P, S, T, L, W, V, or I
  VARIANT 19: note = F or C
  VARIANT 20: note = Any amino acid or absent
  VARIANT 21: note = G, R, F, H, S, D, V, A, Q, P, K, or absent
  VARIANT 22: note = G, Q, R, P, F, S, H, T, M, N, L, or absent
  VARIANT 23: note = Residue may be absent
  VARIANT 24: note = Residue may be absent
GGXXXXXXXXXDHXXXXXXXXXGG

SEQ ID NO: 492 - protein, 20 aa, synthetic construct
  VARIANT 1: note = Residue may be absent
  VARIANT 2: note = Residue may be absent
  VARIANT 3: note = F, H, W, Y, G, or absent
  VARIANT 4: note = Any amino acid
  VARIANT 5: note = L or C
  VARIANT 6: note = I, V, F, Y, W, A, G, P, S, T, L, M, or C
  VARIANT 7: note = N or Q
  VARIANT 10: note = P or G
  VARIANT 11: note = E or W
  VARIANT 12: note = V, F, Y, W, A, G, P, S, T, L, I, or M
  VARIANT 13: note = M, V, F, Y, W, A, G, P, S, T, H, L, or I
  VARIANT 14: note = C or R
  VARIANT 15: note = I, M, F, Y, W, A, G, P, S, T, V, L, or E
  VARIANT 16: note = Any amino acid
  VARIANT 17: note = Any amino acid or absent
  VARIANT 18: note = Any amino acid or absent
  VARIANT 19: note = Residue may be absent
  VARIANT 20: note = Residue may be absent
GGXXXXXDHXXXXXXXXXGG

(19) United States
(12) Patent Application Publication
(10) Pub. No.: US 2024/0158457 A1
(43) Pub. Date: May 16, 2024

(54) INTERLEUKIN-18 RECEPTOR BINDING POLYPEPTIDES AND USES THEREOF

(71) Applicant: Medikine, Inc., Menlo Park, CA (US)

(72) Inventors: William J. DOWER, Menlo Park, CA (US); Ronald William BARRETT, Saratoga, CA (US); Michael C. NEEDELS, El Granada, CA (US); Steven E. CWIRLA, Menlo Park, CA (US); Alice V. BAKKER, Cupertino, CA (US)

(21) Appl. No.: 18/319,903

(22) Filed: May 18, 2023

Related U.S. Application Data
(60) Provisional application No. 63/344,333, filed on May 20, 2022.
(60) Provisional application No. 63/481,026, filed on Jan. 23, 2023.
(60) Provisional application No. 63/489,365, filed on Mar. 9, 2023.

(51) Int. Cl. C07K 14/54

(52) U.S. Cl. CPC C07K 14/54

(57) ABSTRACT

Described herein are interleukin-18 receptor binding moieties and uses thereof. An interleukin-18 receptor binding moiety can be an interleukin-18 receptor agonist or antagonist. An interleukin-18 receptor agonist can be an interleukin-18 receptor alpha binding moiety, an interleukin-18 receptor beta binding moiety, or can include interleukin-18 receptor alpha binding moiety and an interleukin-18 receptor beta binding moiety. An interleukin-18 receptor antagonist can be an interleukin-18 receptor alpha binding moiety, an interleukin-18 receptor beta binding moiety, or can include interleukin-18 receptor alpha binding moiety and an interleukin-18 receptor beta binding moiety. Interleukin-18 receptor agonists and interleukin-18 receptor antagonists can be formulated in a pharmaceutical formulation. Interleukin-18 receptor agonists and interleukin-18 receptor antagonists are useful for treating diseases and disorders in a subject in need thereof

### CROSS SUMMARY

The present application claims the benefit of U.S. Provisional Application No. 63/344,333, filed May 20, 2022, U.S. Provisional Application No. 63/481,026, filed Jan. 23, 2023, and U.S. Provisional Application No. 63/489,365, filed Mar. 9, 2023, each of which is incorporated herein by reference in its entirety.

### SEQUENCE LISTING

The instant application contains a Sequence Listing which has been submitted electronically in XML file format and is hereby incorporated by reference in its entirety. Said XML copy, created on May 12, 2023, is named 50883-702_201 SL.xml and is 638,751 bytes in size.

### SUMMARY OF THE INVENTION

Described herein are polypeptides which comprise an interleukin 18 (IL-18) receptor (IL-18R) binding moiety, wherein the IL-18R binding moiety (a) does not bind to an IL-18 binding protein (IL-18BP) or (b) binds to an IL-18BP with a K_{D} that is at least 1,000-fold higher than the K_{D} of the interaction between the IL-18R binding moiety and IL-18R alpha or IL-18R beta. In some instances, the IL-18R binding moiety is an agonist and agonism is determined by a reporter assay as described in, for example, Example 1. In some instances, the IL-18R binding moiety is an antagonist and antagonism is determined by an assay as described in, for example, Example 5 or 6. In some embodiments, the IL-18R binding moiety can have an EC50 value of less than 300 nM, less than 100 nM, or less than 10 nM. In one aspect, the IL-18R binding moiety is an IL-18R alpha binding moiety. Alternatively, or in addition, the IL-18R binding moiety is an IL-18R beta binding moiety. Alternatively, or in addition, the IL-18R binding moiety contains an IL-18R alpha binding moiety and an IL-18R beta binding moiety. Alternatively, or in addition, the polypeptide can be, for example, from about 8 to about 100, from about 8 to about 90, from about 8 to about 80, from 8 to about 70, from 8 to about 60, from 8 to about 50, from 8 to about 40, from 8 to about 30, or from 8 to about 25 amino acid residues in length. The IL-18R binding moiety can bind to IL-18R alpha with a K D of less than 10 μM. The IL-18R binding moiety can have an IC50 of less than 10 μM. The IL-18R binding moiety can bind to IL-18R alpha with a K_{D} of less than 10 μM and can have an IC50 of less than 10 μM. Alternatively, or in addition, the polypeptide can further include, for example, one or more linker(s). A pharmaceutical formulation can include, for example, (a) the polypeptide (IL-18R agonist or IL-18R antagonist) and (b) a pharmaceutically acceptable excipient. Also described are methods of treating a disease or disorder in a subject in need thereof, comprising administering to the subject one or more of the polypeptide(s) described herein or the pharmaceutical formulation. In one instance, the polypeptide in the formulation is the IL-18R agonist. In another instance, the polypeptide in the formulation is the IL-18R antagonist. The disease or disorder can be, for example, a cancer, an autoimmune disease, an inflammatory disease or disorder, an infectious disease or disorder, a metabolic disease or disorder, a neurodegenerative disease or disorder, a myocardial infarction, emphysema, psoriasis, a hemophagocytic syndrome, a macrophage activation syndrome, sepsis, acute kidney injury, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the instant specification is intended to supersede and/or take precedence over any such contradictory material.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 provides a cartoon representation of IL-18 receptor signal transduction. IL-18 forms a complex by binding to the IL-18 receptor alpha chain (IL-18Rα). The co-receptor IL-18 receptor beta chain (IL-18Rβ) is recruited to form a high affinity complex. Following the formation of a heterodimer, the Toll-IL-1 receptor (TIR) domains trigger binding of MyD88, phosphorylation of the four IRAKs, TRAF-6, and activation of NFκB. In the absence of IL-18β, IL-18 binds to IL-18Rα without inducing a pro-inflammatory signal. The IL-18BP is present in the extracellular compartment where it binds mature IL-18 and prevents binding to the IL-18 receptor.

FIG. 2A illustrates exemplary IL-18R alpha binding polypeptides from sequence family 1a. The figure discloses SEQ ID NOS: 1, 3-14, 2, and 15, respectively, in order of appearance.

FIG. 2B illustrates exemplary IL-18R alpha binding polypeptides from sequence family 1b. The figure discloses SEQ ID NOS: 421-432, respectively, in order of appearance.

FIG. 2C illustrates exemplary IL-18R alpha binding polypeptides from sequence family 1c. The figure discloses SEQ ID NOS: 53-67 and 433, respectively, in order of appearance.

FIG. 3 illustrates exemplary IL-18R alpha binding polypeptides from sequence family 2. The figure discloses SEQ ID NOS: 83-85, respectively, in order of appearance.

FIG. 4 illustrates exemplary IL-18R alpha binding polypeptides from sequence family 3. The figure discloses SEQ ID NOS: 89, 91-92, and 95, respectively, in order of appearance.

FIG. 5 illustrates exemplary IL-18R alpha binding polypeptides from sequence family 4. The figure discloses SEQ ID NOS: 125-126, 128, 97, 434, and 99-110, respectively, in order of appearance.

FIG. 6 illustrates exemplary IL-18R alpha binding polypeptides from sequence family 5. The figure discloses SEQ ID NOS: 130-135, respectively, in order of appearance.

FIG. 7A illustrates exemplary IL-18R alpha binding polypeptides from sequence family 6. The figure discloses SEQ ID NOS: 142-144, 435-436, and 147-151, respectively, in order of appearance.

FIG. 7B illustrates exemplary truncated IL-18R alpha binding polypeptides. The figure discloses SEQ ID NOS: 437-445, respectively, in order of appearance.

FIG. 8 illustrates exemplary IL-18R alpha binding polypeptides from sequence family 7. The figure discloses SEQ ID NOS: 176, 178, and 180-182, respectively, in order of appearance.

FIG. 9 illustrates exemplary IL-18R alpha binding polypeptides from sequence family 8. The figure discloses SEQ ID NOS: 198, 196, 199, and 186-190, respectively, in order of appearance.

FIG. 10 illustrates exemplary IL-18R beta binding polypeptides from sequence family 1. The figure discloses SEQ ID NOS: 207-211, 201, 203-204, and 446-447, respectively, in order of appearance.

FIG. 11A illustrates exemplary IL-18R beta binding polypeptides from sequence family 2. The figure discloses SEQ ID NOS: 295-296, 277-285, and 219-231, respectively, in order of appearance.

FIG. 11B illustrates exemplary IL-18R beta binding polypeptides from sequence family 2. The figure discloses SEQ ID NOS: 232-247 and 217, respectively, in order of appearance.

FIG. 12A illustrates exemplary IL-18R beta binding polypeptides from sequence family 3. The figure discloses SEQ ID NOS: 355, 357, 341-347, and 300-314, respectively, in order of appearance.

FIG. 12B illustrates exemplary IL-18R beta binding polypeptides from sequence family 3. The figure discloses SEQ ID NOS: 315, 299, 316-319, 300, 448, and 302-304, respectively, in order of appearance.

FIG. 13 illustrates exemplary IL-18R beta binding polypeptides from sequence family 4. The figure discloses SEQ ID NOS: 359, 207, and 361, respectively, in order of appearance.

FIG. 14 illustrates exemplary IL-18R beta binding polypeptides from sequence family 5. The figure discloses SEQ ID NOS: 382, 364-373, 203, 374-376, 449, and 378-381, respectively, in order of appearance.

FIGS. 15A-15D provide IC50 data from exemplary IL-18R alpha binding polypeptides 1E11 (FIG. 15A) and 7C06 (FIG. 15B) and from exemplary IL-18R beta binding polypeptides Family 2 synthetic construct (FIG. 15C) and 5C09 (FIG. 15D).

FIGS. 16A-16G are exemplary diagrams of dimer structures of IL-18R antagonists Dimer 5 (FIG. 16A), Dimer 6 (FIG. 16B), Dimer 7 (FIG. 16C), Dimer 10 (FIG. 16D), Dimer 11 (FIG. 16E), Dimer 12 (FIG. 16F), and Dimer 13 (FIG. 16G). The figure discloses SEQ ID NOS: 450-451, 450, 411, 450, 409, 452, 418, 450, 450, 482, 453, 450, and 481, respectively, in order of appearance.

FIGS. 17A-17B are exemplary diagrams of dimer structures of IL-18R antagonists Dimer 8 (FIG. 17A) and Dimer 9 (FIG. 17B). The figure discloses SEQ ID NOS: 412, 411, 412, and 409, respectively, in order of appearance.

FIGS. 18A-18D provide IC50 data determined by competition ELISA for IL-18R alpha Family F1c synthetic construct (FIG. 18A), clone 10D09 (FIG. 18B), clone 17G07 (FIG. 18C), and clone 11G01 (FIG. 18D).

FIGS. 19A-19J provide IC50 data determined by inhibition of IFN gamma (IFNg) secretion in KG-1 cells for clone 10D09 (FIG. 19A), clone 17G07 (FIG. 19B), Dimer 5 (FIG. 19C), Dimer 8 (FIG. 19D; C-N orientation), Dimer 6 (FIG. 19E; N-N orientation), Dimer 7 (FIG. 19F; N-C orientation), Dimer 9 (FIG. 19G; C-C orientation), Dimer 11 (FIG. 19H; N-N orientation), Dimer 12 (FIG. 19I; N-N orientation), and Dimer 13 (FIG. 19J; N-N orientation).

FIG. 20A provides interferon gamma (IFNγ) levels produced by primary NK cells after an 8-hour exposure to 10 μM of IL-18R binding polypeptides in the presence of IL-18 (10 ng/ml) and IL-12 (12.5 ng/ml) compared to cells exposed to cytokine alone. Bars marked with * show a decrease in IFNγ levels upon addition of the indicated compound. The dotted line is the signal produced from cells stimulated with IL-18 plus IL-12 without antagonist peptides. Unstim: unstimulated.

FIG. 20B provides IFNγ levels produced by primary NK cells after an 8-hour exposure to 1 μM of IL-18R binding polypeptides (unless indicated otherwise) in the presence of IL-18 (10 ng/ml) and IL-12 (12.5 ng/ml) compared to cells exposed to cytokine alone. Bars marked with * show a decrease in IFNγ levels upon addition of the indicated compound. The dotted line is the signal produced from cells stimulated with IL-18 plus IL-12 without antagonist peptides. Unstim: unstimulated.

FIG. 20C provides IFNγ levels produced by primary NK cells after an 8-hour exposure to IL-18R antagonists in the presence of IL-18 (10 ng/ml) and IL-12 (12.5 ng/ml) compared to cells exposed to cytokine alone. Bars marked with * show a decrease in IFNγ levels upon addition of the indicated compound. Unstim: unstimulated.

FIGS. 21A-E demonstrate binding affinity data for IL-18BP for biotinylated 4D07 (bn4D07; FIG. 21A), biotinylated 7C06 (bn7C06; FIG. 21B), biotinylated alpha synthetic F1a Consensus Construct (bn-alpha synthetic F1a Consensus Construct; FIG. 21C), biotinylated beta family 2 synthetic construct (bn-beta family 2 synthetic construct; FIG. 21D), and 5C09 (FIG. 21E).

FIGS. 22A-B demonstrate IL18BP and dimer 10 titration. IL-18 and dimer 10 are agonists of IL-18R with EC50s of 0.38 pM and 28 nM, respectively (FIG. 22A). Pre-incubated titration of binding protein (10×; 100 nM starting) with either 1.5 pM IL-18 or 30 nM dimer 10 for ˜30 mins (concentrations noted are final after addition to cells). Added 20 !alto 180 μL of cells (at approx. 3E5/m1).The IL-18BP was confirmed to inhibit bioactivity of IL-18 with an IC50 of about 1 nM, and IL-18BP does not inhibit the agonist activity of dimer 10 up to 100 nM (FIG. 22B).

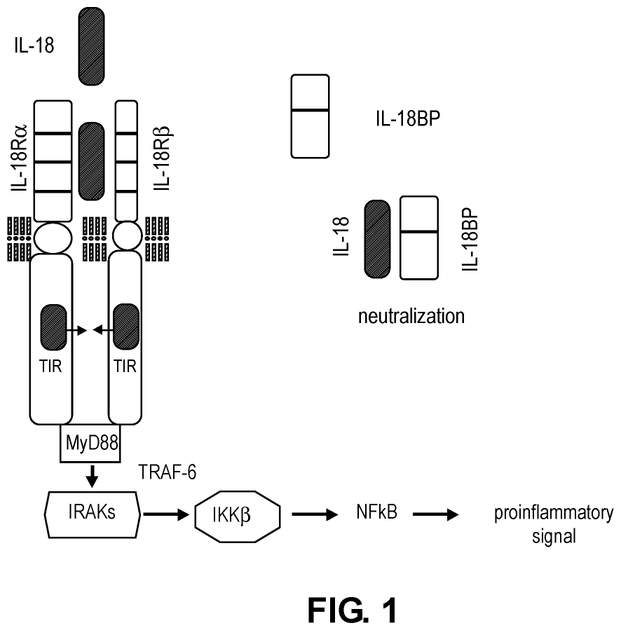

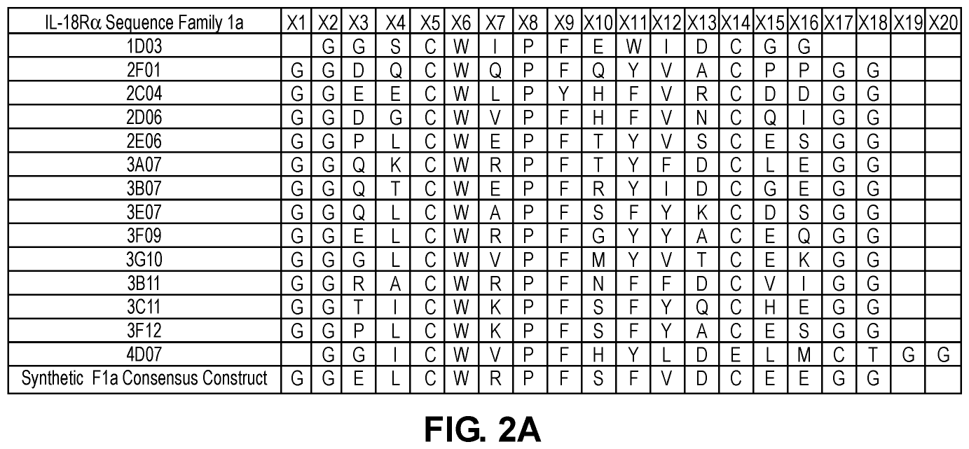

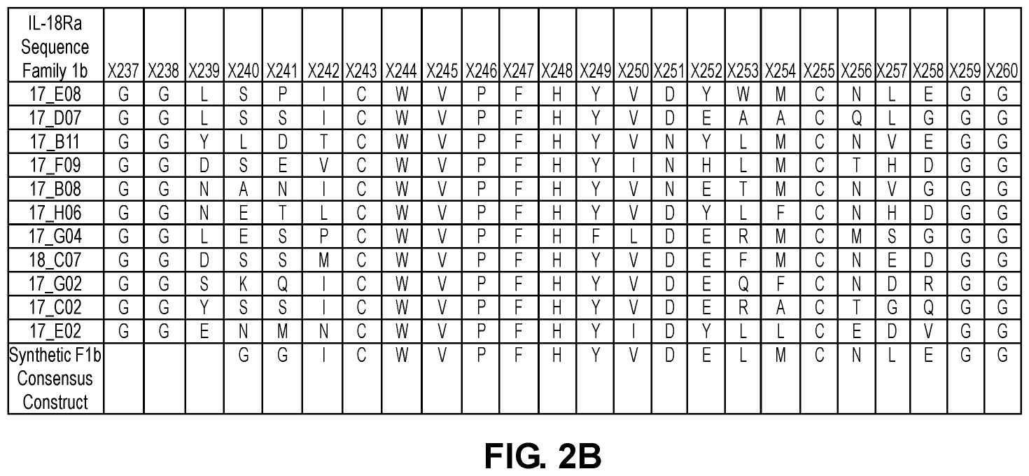

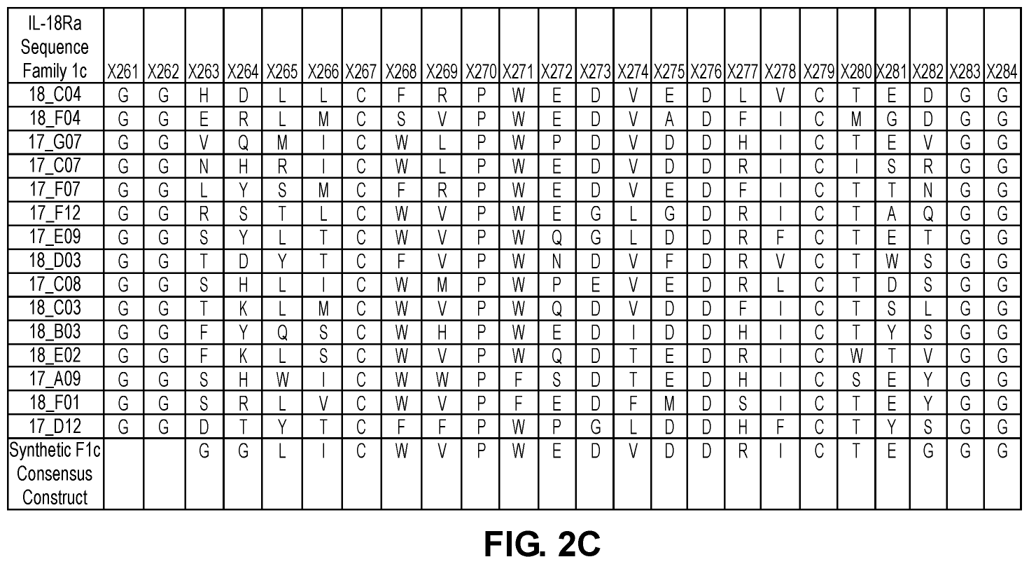

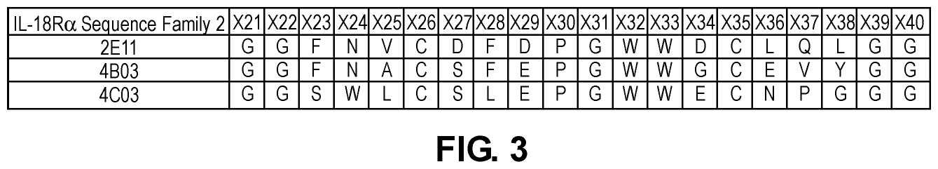

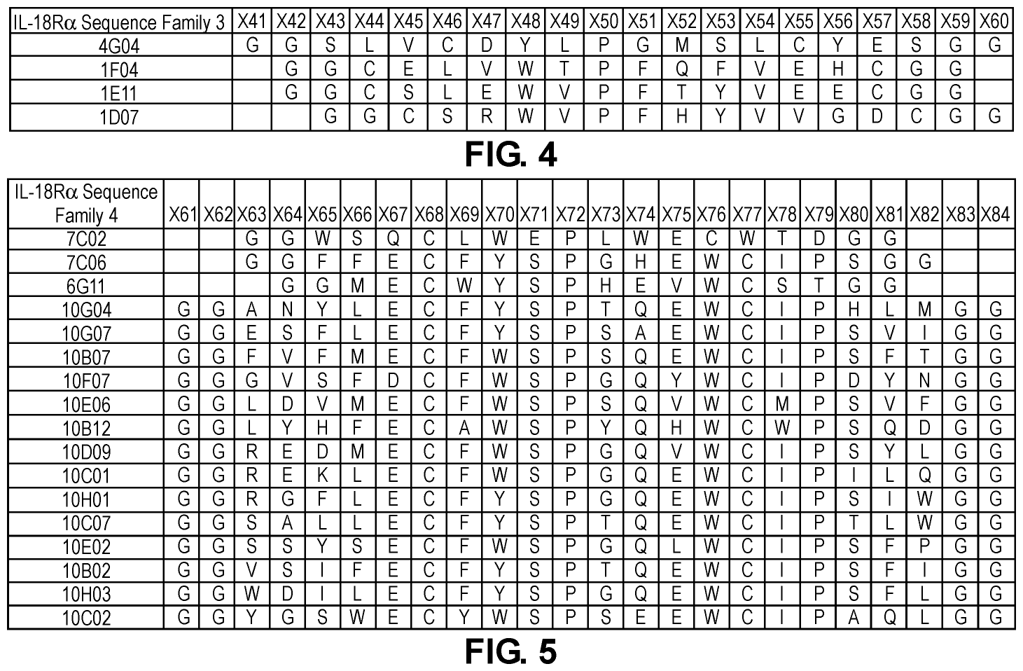

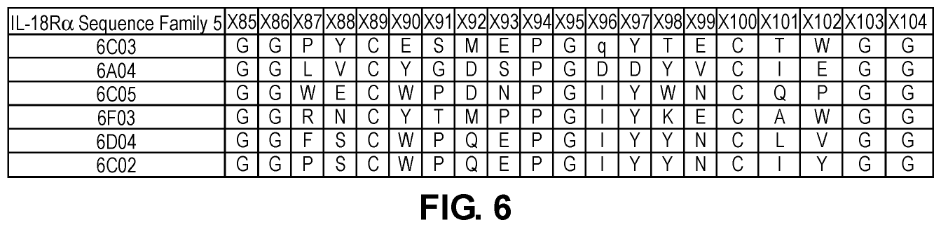

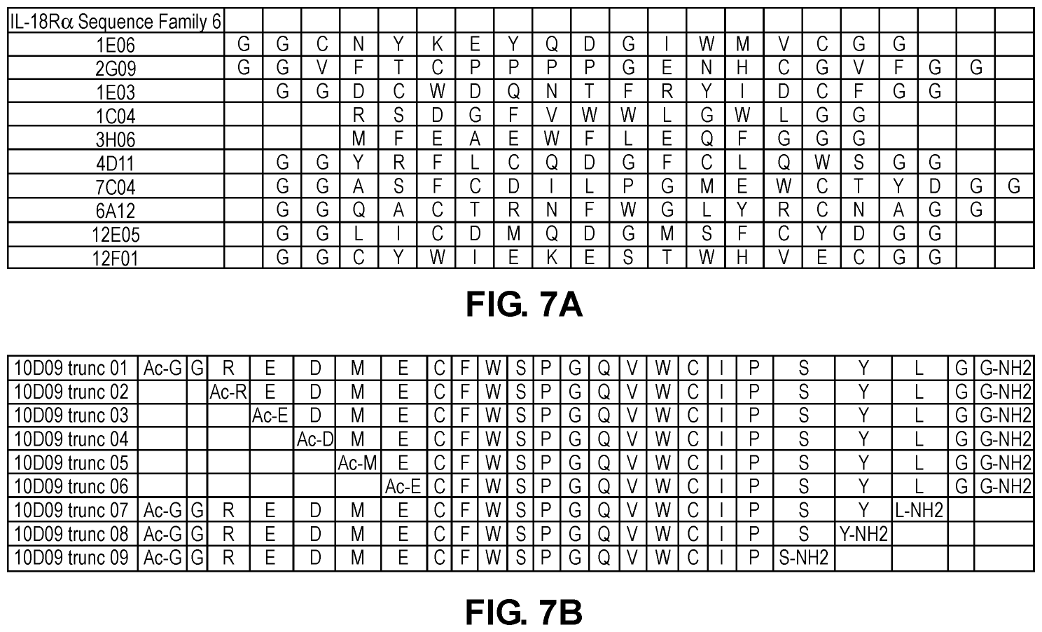

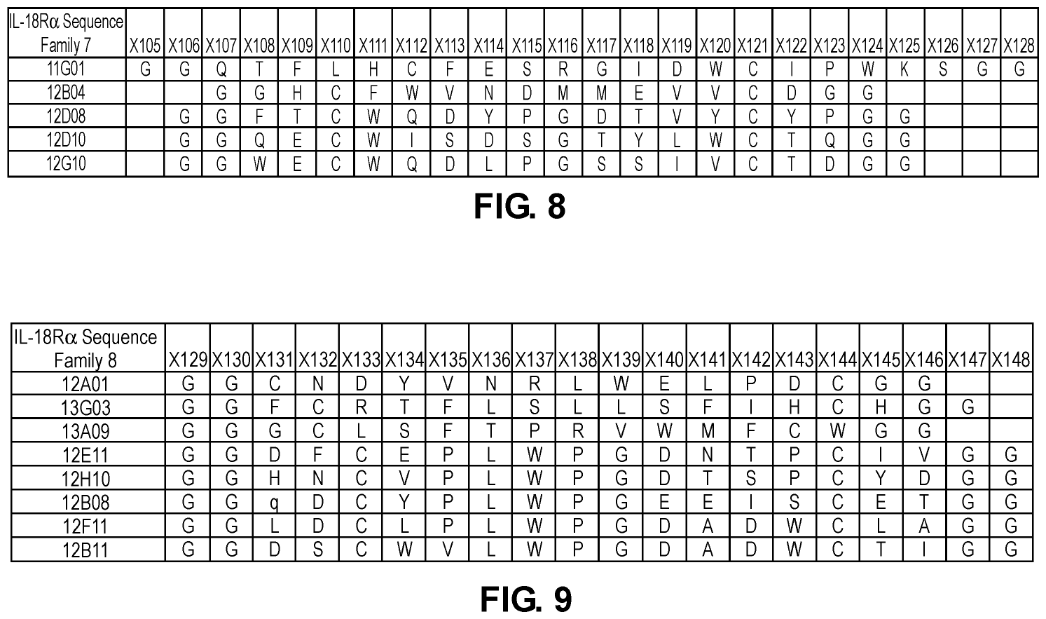

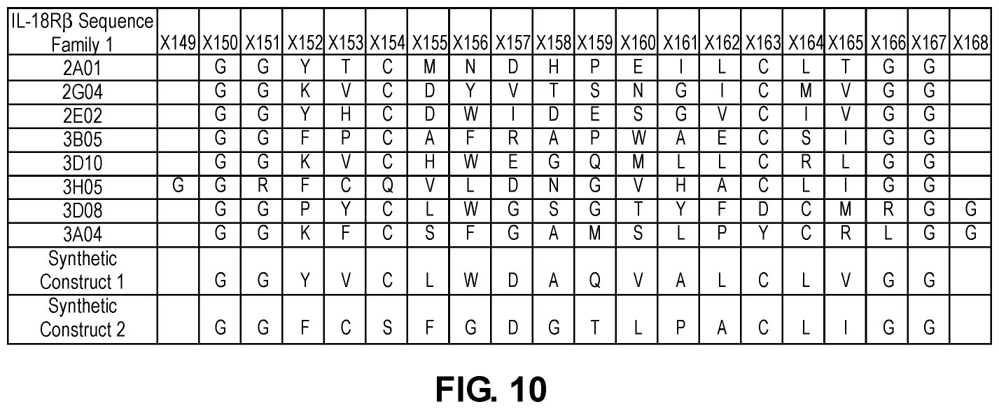

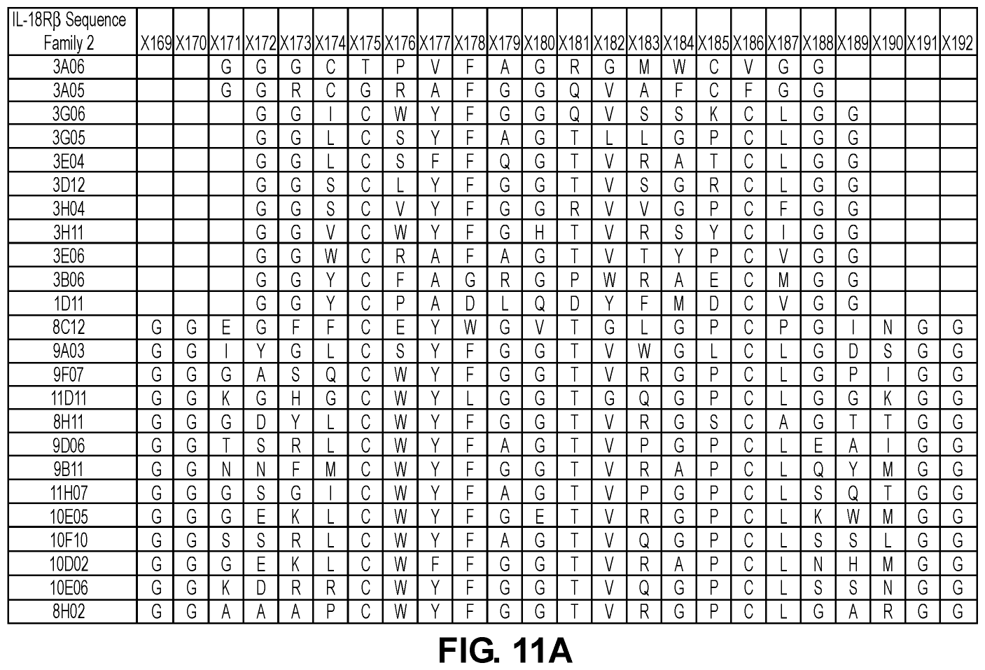

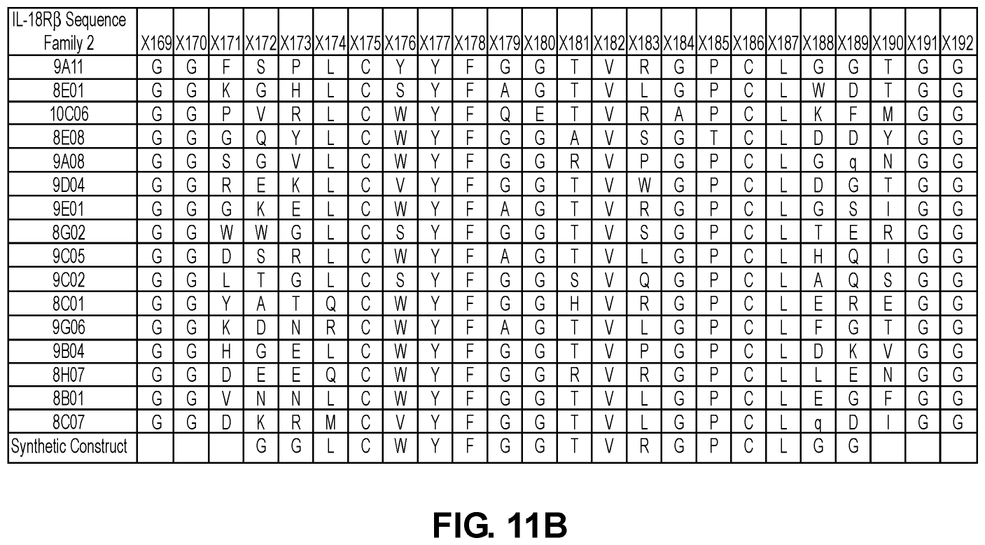

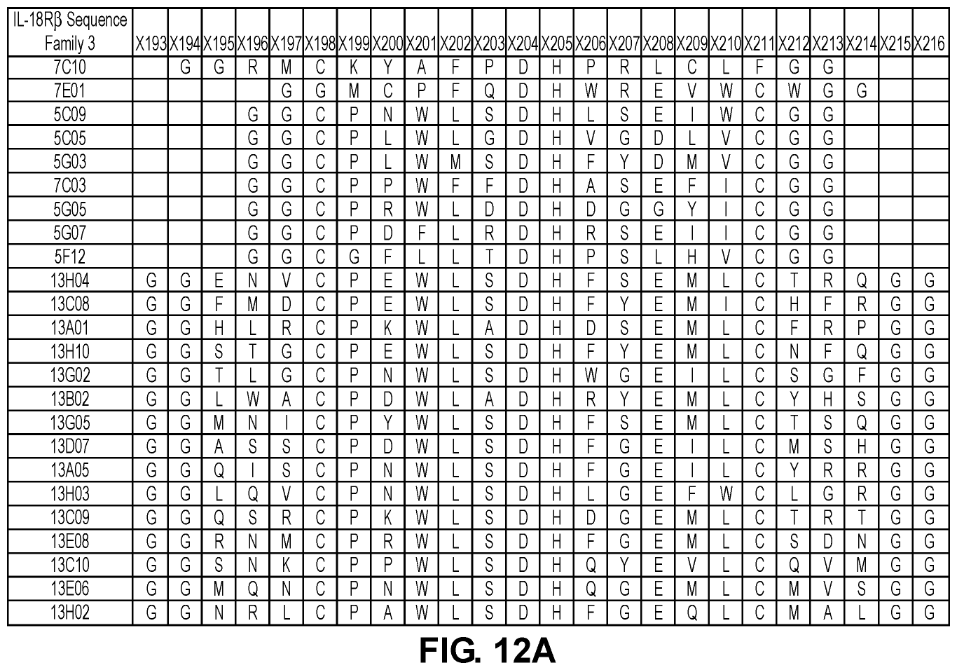

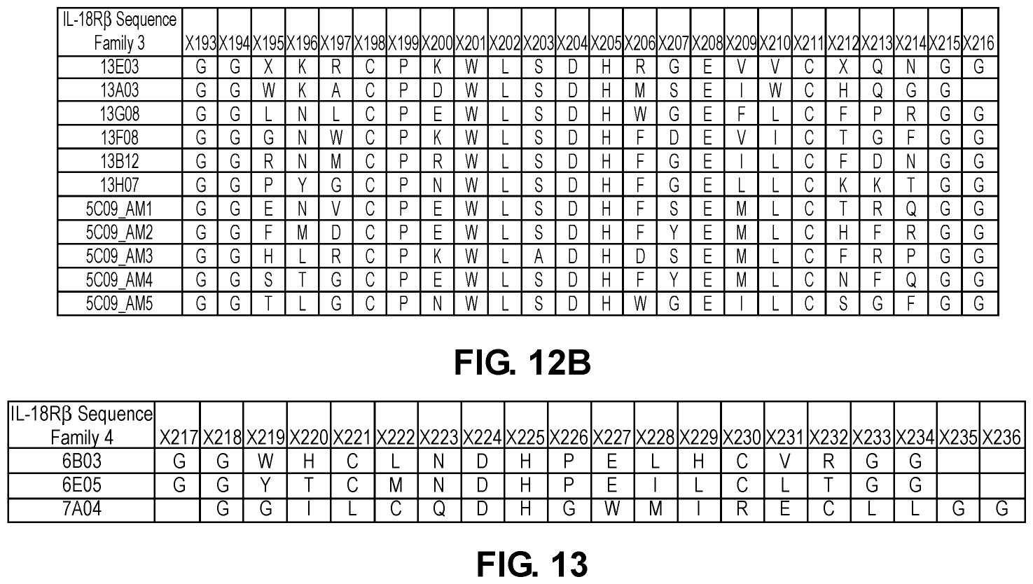

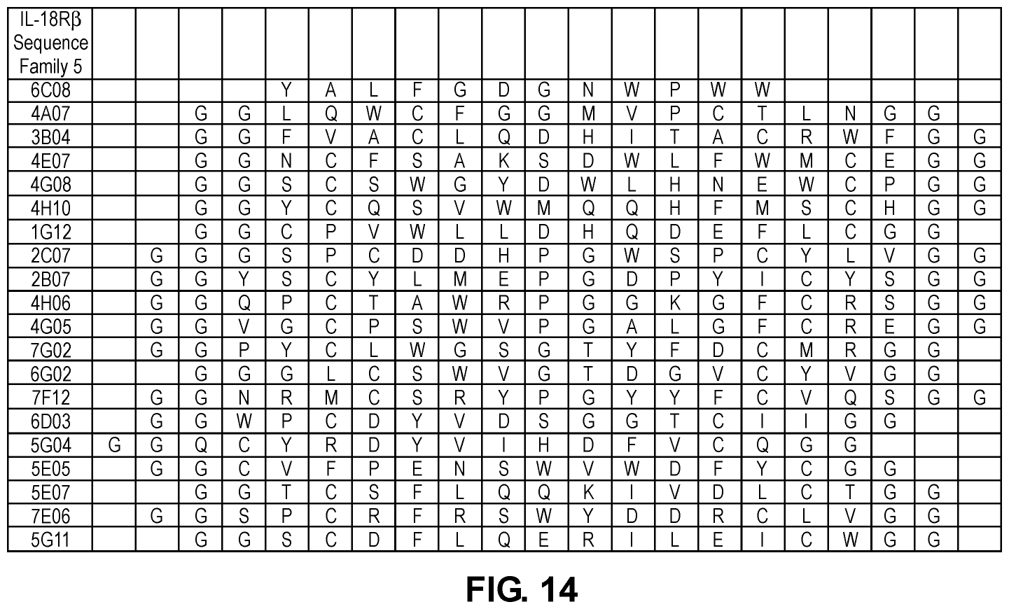

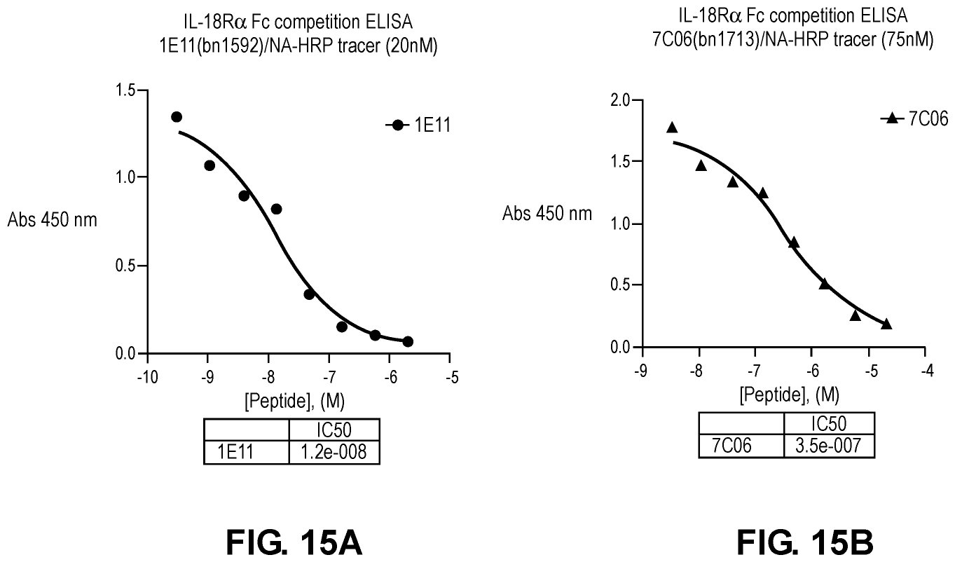

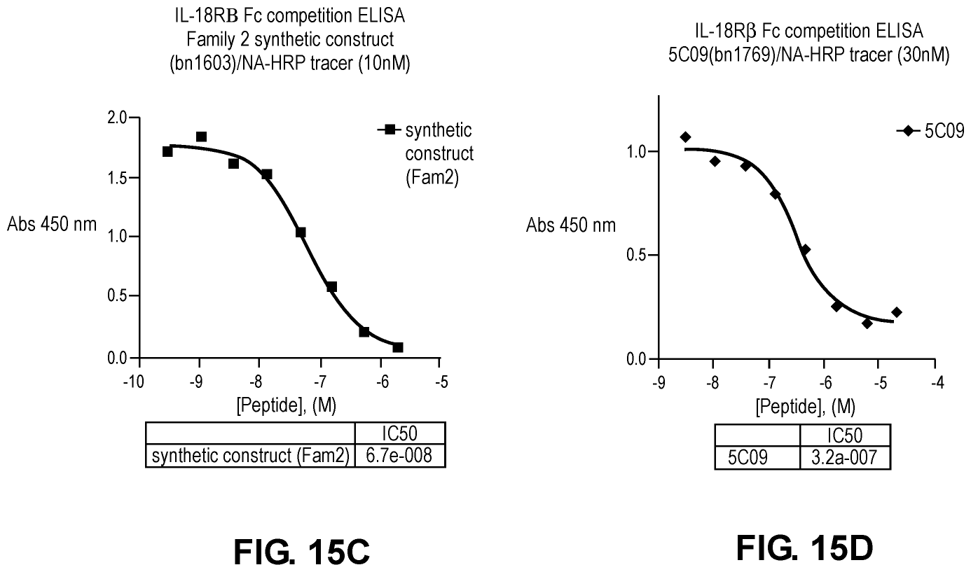

### DETAILED DESCRIPTION OF THE INVENTION

Interleukin-18 (IL-18) is a member of the IL-1 family of cytokines. IL-18 is synthesized as an inactive precursor and is processed by caspase-1 into an active cytokine. The IL-18 precursor is constitutively present in the majority of cells in healthy humans and animals. IL-18 is first synthesized as an inactive precursor without a signal peptide and remains as an intracellular cytokine. The IL-18 precursor is present in blood monocytes from healthy subjects and in the epithelial cells of the entire gastrointestinal tract. Peritoneal macrophages and mouse spleen also contain the IL-18 precursor in the absence of disease. The IL-18 precursor is also present in keratinocytes and nearly all epithelial cells. The activity of IL-18 is kept in balance by the presence of a high affinity, naturally occurring IL-18 binding protein (IL-18BP). In humans, increased disease severity can be associated with an imbalance of IL-18 to IL-18BP and can prevent IL-18 receptor agonists from exerting a therapeutic effect.

IL-18 is involved in the Th1 paradigm due to induction of IFNγ with either IL-12 or IL-15. Without IL-12 or IL-15, IL-18 does not induce IFNγ. IL-12 or IL-15 increases the expression of IL-18Rβ, which induces IL-18 signal transduction when co-localized with IL-18Ra. In the absence of IL-12 or IL-15, IL-18 plays a role in Th2 diseases. The role of IL-18 as an immunoregulatory cytokine is a result of its function of inducing IFNγ from NK cells. Macrophage colony stimulating factor (M-CSF) induces human blood monocytes to differentiate into a subset of macrophages; these cells express a membrane-bound form of IL-18. Upon shedding of membrane IL-18 into a soluble form, NK cells express CCR7 and produce high levels of IFNγ.

IL-18BP is a secreted protein, with a high affinity for IL-18 (e.g., approx. 400 pM) and serves as a decoy receptor. IL-18BP can be present in the serum of healthy humans at a 20-fold molar excess, or more, compared to IL-18. IL-18BP contains only one IgG domain. IL-18BP down-regulates Th1 immune responses by binding to IL-18, thereby reducing IFNγ induction. Since IL-18 also affects Th2 responses, IL-18BP also has properties controlling a Th2 cytokine response. IFNγ increases gene expression and synthesis of IL-18BP, which creates a negative feedback loop. Treatment with IL-18 receptor agonists is hindered by the inhibitory actions of IL-18BP.

### IL-18R Agonists and Antagonists

Described herein are polypeptides which comprise an interleukin 18 (IL-18) receptor (IL-18R) binding moiety, wherein the IL-18R binding moiety (a) does not bind to the IL-18BP or (b) binds to the IL-18BP with a K_{D} that is at least 1,000-fold higher than the K_{D} of the interaction between the IL-18R binding moiety and IL-18R alpha or IL-18R beta. In some instances, the IL-18R binding moiety is an agonist, and agonism is determined by a reporter assay as described in, for example, Example 1. In some instances, the IL-18R binding moiety is an antagonist, and antagonism is determined by an assay as described in, for example, any one of Examples 6-7. In one aspect, the IL-18R binding moiety is an IL-18R alpha binding moiety. Alternatively, or in addition, the IL-18R binding moiety is an IL-18R beta binding moiety. Alternatively, or in addition, the IL-18R binding moiety contains an IL-18R alpha binding moiety and an IL-18R beta binding moiety. Alternatively, or in addition, the polypeptide can be, for example, from about 10 to about 100 amino acid residues in length. The polypeptide can have a K_{D} of less than 10 μM. The polypeptide can have an IC50 of less than 10 μM. Alternatively, or in addition, the polypeptide can further include, for example, one or more linker(s). Alternatively, or in addition, the polypeptide can be modified to add (e.g., include) two N-terminal ‘G’ residues or two C-terminal ‘G’ residues.

An IL-18R binding moiety can be further modified. For example, an IL-18R binding moiety can be modified to attach (e.g., covalently) a sterically bulky moiety to improve or enhance activity where such a sterically bulky moiety can comprise a protein, peptide, or non-peptidic chemical entity. Alternatively, or in addition, an IL-18R binding moiety can be a bispecific agent which comprises (a) an IL-18R binding moiety and (b) a binding moiety that specifically binds to a different target and may or may not have either agonist or antagonist activity towards the different target.

### IL-18R Alpha Binding Moieties

In one aspect, provided herein is an IL-18R binding moiety comprising an IL-18R alpha binding moiety. An IL-18R alpha binding moiety can, in some instances, be an IL-18R agonist. Alternatively, an IL-18R alpha binding moiety can, in other instances, be an IL-18R antagonist. Each of the IL-18R alpha binding moieties were found to bind to IL-18R alpha with a binding affinity (K_{D} or IC50) of less than 10 uM (data not shown). The IL-18R alpha binding moiety either (a) does not bind to the IL-18BP or (b) binds to the IL-18BP with a K_{D} that is at least 1,000-fold higher than the K_{D} of the interaction between the IL-18R binding moiety and IL-18R alpha. In one instance, the IL-18R alpha binding moiety does not bind to the IL-18BP. In another instance, the IL-18R alpha binding moiety binds to the IL-18BP with a K_{D} that is at least 1,000-fold higher than the K_{D} of the interaction between the IL-18R alpha binding moiety and IL-18R alpha.

### IL-18R alpha Sequence Family 1a

In one aspect, an IL-18R alpha binding moiety can have the amino acid sequence of X1-X2-X3-X4-X5-X6-X7-X8-X9-X10-X11-X12-X13-X14-X15-X16-X17-X18-X19-X20, wherein: (i) one or more of X1, X2, X3, X17, X18, X19, and X20 are optional, (ii) if present, X1 is G; if present, X2 is G; if present, X3 is Xaa; X4 is S, Q, E, G, L, K, T, A, or I; X5 is C; X6 is W; X7 is I, Q, L, V, E, R, A, or K; X8 is P; X9 is F or Y; X10 is E, Q, H, T, R, S, G, M, or N; X11 is H, W, Y, or F; X12 is I, L, M, V, F, Y, W, A, G, P, S, or T; X13 is D, A, R, N, S, K, T, or Q; X14 is C or E; X15 is G, P, D, Q, E, L, V, or H; X16 is G, P, D, I, S, E, Q, K, I, or M; if present, X17 is G or C; if present, X18 is G or T; if present, X19 is G; and if present, X20 is G; and (iii) Xaa is any amino acid (SEQ ID NO: 454). In one embodiment, X3 is Xaa; X4 is L or I; X5 is C; X6 is W; X7 is R or K; X8 is P; X9 is F; X10 is S or T; X11 is F, H, W, or Y; X12 is I, L, M, V, F, Y, W, A, G, P, S, or T; X13 is D; X14 is C; X15 is E or D; and X16 is E, D, or Q (F1 consensus; SEQ ID NO: 455). In another embodiment, Xl, X17, X18, X19, and X20 are absent and examples of the IL-18R alpha binding moiety include, but are not limited to, (i) the amino acid sequence of GGSCWIPFEWIDCGG (1D03; SEQ ID NO: 1) or (ii) an amino acid sequence having 1, 2, 3, or 4 substitution(s), addition(s), and/or deletion(s) relative to the amino acid sequence of (i). In another embodiment, X1 is absent and examples of the IL-18R alpha binding moiety include, but are not limited to, (i) the amino acid sequence of GGICWVPFHYLDELMCTGG (4D07; SEQ ID NO: 2) or (ii) an amino acid sequence having 1, 2, 3, or 4 substitution(s), addition(s), and/or deletion(s) relative to the amino acid sequence of (i). In another embodiment, X19 and X20 are absent and examples of the IL-18R alpha binding moiety include, but are not limited to, (i) the amino acid sequence of GGDQCWQPFQYVACPPGG (2F01; SEQ ID NO: 3); GGEECWLPYHFVRCDDGG (2C04; SEQ ID NO: 4); GGDGCWVPFHFVNCQIGG (2D06; SEQ ID NO: 5); GGPLCWEPFTYVSCESGG (2E06; SEQ ID NO: 6); GGQKCWRPFTYFDCLEGG (3A07; SEQ ID NO: 7); GGQTCWEPFRYIDCGEGG (3B07; SEQ ID NO: 8); GGQLCWAPFSFYKCDSGG (3E07; SEQ ID NO: 9); GGELCWRPFGYYACEQGG (3F09; SEQ ID NO: 10); GGGLCWVPFMYVTCEKGG (3G10; SEQ ID NO: 11); GGRACWRPFNFFDCVIGG (3B11; SEQ ID NO: 12); GGTICWKPFSFYQCHEGG (3C11; SEQ ID NO: 13); GGPLCWKPFSFYACESGG (3F12; SEQ ID NO: 14); or GGELCWRPFSFVDCEEGG (alpha synthetic F1 peptide; SEQ ID NO: 15); or (ii) an amino acid sequence having 1, 2, 3, or 4 substitution(s), addition(s), and/or deletion(s) relative to an amino acid sequence of any one of (i). In another embodiment, X1, X2, X3, X15, X16, X17, X18, X19, and X20 are absent and examples of the IL-18R alpha binding moiety include, but are not limited to, (i) the amino acid sequence of SCWIPFEWIDC (1D03-G; SEQ ID NO: 16) or (ii) an amino acid sequence having 1, 2, 3, or 4 substitution(s), addition(s), and/or deletion(s) relative to the amino acid sequence of (i). In another embodiment, X1, X2, X3, X19, and X20 are absent and examples of the IL-18R alpha binding moiety include, but are not limited to, (i) the amino acid sequence of ICWVPFHYLDELMCT (4D07-G; SEQ ID NO: 17) or (ii) an amino acid sequence having 1, 2, 3, or 4 substitution(s), addition(s), and/or deletion(s) relative to the amino acid sequence of (i). In another embodiment, X1, X2, X17, X18, X19, and X20 are absent and examples of the IL-18R alpha binding moiety include, but are not limited to, (i) the amino acid sequence of DQCWQPFQYVACPP (2F01-G; SEQ ID NO: 18); EECWLPYHFVRCDD (2C04-G; SEQ ID NO: 19); DGCWVPFHFVNCQI (2D06-G; SEQ ID NO: 20); PLCWEPFTYVSCES (2E06-G; SEQ ID NO: 21); QKCWRPFTYFDCLE (3A07-G; SEQ ID NO: 22); QTCWEPFRYIDCGE (3B07-G; SEQ ID NO: 23); QLCWAPFSFYKCDS (3E07-G; SEQ ID NO: 24); ELCWRPFGYYACEQ (3F09-G; SEQ ID NO: 25); GLCWVPFMYVTCEK (3G10-G; SEQ ID NO: 26); RACWRPFNFFDCVI (3B11-G; SEQ ID NO: 27); TICWKPFSFYQCHE (3C11-G; SEQ ID NO: 28); PLCWKPFSFYACES (3F12-G; SEQ ID NO: 29); or ELCWRPFSFVDCEE (alpha synthetic F1 peptide-G; SEQ ID NO: 30); or (ii) an amino acid sequence having 1, 2, 3, or 4 substitution(s), addition(s), and/or deletion(s) relative to an amino acid sequence of any one of (i). In another embodiment, X1 is G; X2 is G; X3 is E; X4 is L; X5 is C; X6 is W; X7 is R; X8 is P; X9 is F; X10 is S; X11 is F; X12 is V; X13 is D; X14 is C; X15 is E; X16 is E; X17 is G; and X18 is G (alpha family la synthetic construct; SEQ ID NO: 456).

### IL-18R Alpha Sequence Family 1b

In another aspect, an IL-18R alpha binding moiety can have the amino acid sequence of X237-X238-X239-X240-X241-X242-X243-X244-X245-X246-X247-X248-X249-X250-X251-X252-X253-X254-X255-X256-X257-X258-X259-X260, wherein: (i) one or more of X237, X238, X259, and X260 are optional, (ii) if present, X237 is G; if present, X238 is G; X239 is L, Y, D, N, S, or E; X240 is S, L, A, E, K, N, or G; X241 is P, S, D, E, N, T, Q, M, or G; X242 is I, T, V, L, P, M, N, F, Y, W, A, G, or S; X243 is C; X244 is W; X245 is V; X246 is P; X247 is F; X248 is H; X249 is Y, or F; X250 is I, L, M, V, F, Y, W, A, G, P, S, or T; X251 is D, or N; X252 is Y, E, or H; X253 is W, A, L, T, R, F, Q, or Xaa; X254 is M, A, F, L, I, V, Y, W, G, P, S, T; X255 is C; X256 is N, Q, T, M, or E; X257 is L, V, H, S, E, D, G, or Xaa; X258 is E, G, D, R, Q, or V; if present, X259 is G; and if present, X260 is G; and (iii) Xaa is any amino acid (SEQ ID NO: 472). In one embodiment, X242 is I, L, M, V, F, Y, W, A, G, P, S, or T; X243 is C; X244 is W; X245 is V; X246 is P; X247 is F; X248 is H; X249 is Y; X250 is V; X251 is D; X252 is E; X253 is Xaa; X254 is M; X255 is C; X256 is N; X257 is Xaa; and X258 is D or E (consensus (a) F1b; SEQ ID NO: 473). In another embodiment, X250 is I, L, M, V, F, Y, W, A, G, P, S, or T; X251 is N; X252 is Y; and X254 is I, L, M, V, F, Y, W, A, G, P, S, or T (consensus (b) F1b; SEQ ID NO: 474). In another embodiment, X240 is G; X241 is G; X242 is I; X243 is C; X244 is W; X245 is V; X246 is P; X247 is F; X248 is H; X249 is Y; X250 is V; X251 is D; X252 is E; X253 is L; X254 is M; X255 is C; X256 is N; X257 is L; X258 is E; X259 is G; and X260 is G (alpha family 1b synthetic construct; SEQ ID NO: 475). In another embodiment, X237, X238, X259, and X260 are present and examples of the IL-18R alpha binding moiety include, but are not limited to, (i) the amino acid sequence of GGLSPICWVPFHYVDYWMCNLEGG (17E08; SEQ ID NO: 31); GGLSSICWVPFHYVDEAACQLGGG (17D07; SEQ ID NO: 32); GGYLDTCWVPFHYVNYLMCNVEGG (17B11; SEQ ID NO: 33); GGDSEVCWVPFHYINHLMCTHDGG (17F09; SEQ ID NO: 34); GGNANICWVPFHYVNETMCNVGGG (17B08; SEQ ID NO: 35); GGNETLCWVPFHYVDYLFCNHDGG (17H06; SEQ ID NO: 36); GGLESPCWVPFHFLDERMCMSGGG (17G04; SEQ ID NO: 37); GGDSSMCWVPFHYVDEFMCNEDGG (18C07; SEQ ID NO: 38); GGSKQICWVPFHYVDEQFCNDRGG (17G02; SEQ ID NO: 39); GGYSSICWVPFHYVDERACTGQGG (17C02; SEQ ID NO: 40); or GGENMNCWVPFHYIDYLLCEDVGG (17E02; SEQ ID NO: 41); or (ii) an amino acid sequence having 1, 2, 3, or 4 substitution(s), addition(s), and/or deletion(s) relative to the amino acid sequence of (i). In another embodiment, X237, X238, X259, and X260 are absent and examples of the IL-18R alpha binding moiety include, but are not limited to, (i) the amino acid sequence of LSPICWVPFHYVDYWMCNLE (17E08-G; SEQ ID NO: 42); LSSICWVPFHYVDEAACQLG (17D07-G; SEQ ID NO: 43); YLDTCWVPFHYVNYLMCNVE (17B11-G; SEQ ID NO: 44); DSEVCWVPFHYINHLMCTHD (17F09-G; SEQ ID NO: 45); NANICWVPFHYVNETMCNVG (17B08-G; SEQ ID NO: 46); NETLCWVPFHYVDYLFCNHD (17H06-G; SEQ ID NO: 47); LESPCWVPFHFLDERMCMSG (17G04-G; SEQ ID NO: 48); DSSMCWVPFHYVDEFMCNED (18C07-G; SEQ ID NO: 49); SKQICWVPFHYVDEQFCNDR (17G02-G; SEQ ID NO: 50); YSSICWVPFHYVDERACTGQ (17C02-G; SEQ ID NO: 51); or ENMNCWVPFHYIDYLLCEDV (17E02-G; SEQ ID NO: 52); or (ii) an amino acid sequence having 1, 2, 3, or 4 substitution(s), addition(s), and/or deletion(s) relative to the amino acid sequence of (i).

### IL-18R Alpha Sequence Family 1c

In another aspect, an IL-18R alpha binding moiety can have the amino acid sequence of X261-X262-X263-X264-X265-X266-X267-X268-X269-X270-X271-X272-X273-X274-X275-X276-X277-X278-X279-X280-X281-X282-X283-X284, wherein: (i) one or more of X261, X262, X283, and X284 are optional, (ii) if present, X261 is G; if present, X262 is G; X263 is H, E, V, N, L, R, S, T, F, D, or Xaa; X264 is D, R, Q, H, Y, S, K, T, or Xaa; X265 is L, M, R, S, T, Y, Q, W, I, V, F, A, G, or P; X266 is L, M, I, T, S, V, F, Y, W, A, G, or P; X267 is C; X268 is F, S, or W; X269 is R, V, L, M, H, W, F, I, Y, A, G, P, S, or T; X270 is P; X271 is W, or F; X272 is E, P, Q, N, or S; X273 is D, G, or E; X274 is V, L, I, T, F, M, Y, W, A, G, P, or S; X275 is E, A, D, G, F, or M; X276 is D; X277 is L, F, H, R, or S; X278 is V, I, F, L, M, Y, W, A, G, P, S, or T; X279 is C; X280 is T, M, I, W, or S; X281 is E, G, S, T, A, W, D, or Y; X282 is D, V, R, N, Q, T, S, L, Y, or Xaa; if present, X283 is G; if present, X284 is G; and (iii) Xaa is any amino acid (SEQ ID NO: 476). In one embodiment, X263 is Xaa; X264 is Xaa; X265 is L; X266 is I, L, M, V, F, Y, W, A, G, P, S, or T; X267 is C; X268 is W; X269 is V; X270 is P; X271 is W; X272 is E; X273 is D; X274 is V; X275 is D or E; X276 is D; X277 is R; X278 is I; X279 is C; X280 is T; X281 is E; and X282 is Xaa (consensus (a) F1c; SEQ ID NO: 477). In another embodiment, X265 is I, L, M, V, F, Y, W, A, G, P, S, or T; X268 is F; X269 is I, L, M, V, F, Y, W, A, G, P, S, or T; X272 is Q or N; X274 is I, L, M, V, F, Y, W, A, G, P, S, or T; X277 is H; X278 is I, L, M, V, F, Y, W, A, G, P, S, or T; and X281 is E, G, S, T, A, W, D, or Y (consensus (b) F1c; SEQ ID NO: 478). In another embodiment, X263 is G; X264 is G; X265 is L; X266 is I; X267 is C; X268 is W; X269 is V; X270 is P; X271 is W; X272 is E; X273 is D; X274 is V; X275 is D; X276 is D; X277 is R; X278 is I; X279 is C; X280 is T; X281 is E; X282 is G; X283 is G; and X284 is G (alpha family 1c synthetic construct; SEQ ID NO: 479). In another embodiment, X261, X262, X283, and X284 are present and examples of the IL-18R alpha binding moiety include, but are not limited to, (i) the amino acid sequence of GGHDLLCFRPWEDVEDLVCTEDGG (18C04; SEQ ID NO: 53); GGERLMCSVPWEDVADFICMGDGG (18F04; SEQ ID NO: 54); GGVQMICWLPWPDVDDHICTEVGG (17G07; SEQ ID NO: 55); GGNHRICWLPWEDVDDRICISRGG (17C07; SEQ ID NO: 56); GGLYSMCFRPWEDVEDFICTTNGG (17F07; SEQ ID NO: 57); GGRSTLCWVPWEGLGDRICTAQGG (17F12; SEQ ID NO: 58); GGSYLTCWVPWQGLDDRFCTETGG (17E09; SEQ ID NO: 59); GGTDYTCFVPWNDVFDRVCTWSGG (18D03; SEQ ID NO: 60); GGSHLICWMPWPEVEDRLCTDSGG (17C08; SEQ ID NO: 61); GGTKLMCWVPWQDVDDFICTSLGG (18C03; SEQ ID NO: 62); GGFYQSCWHPWEDIDDHICTYSGG (18B03; SEQ ID NO: 63); GGFKLSCWVPWQDTEDRICWTVGG (18E02; SEQ ID NO: 64); GGSHWICWWPFSDTEDHICSEYGG (17A09; SEQ ID NO: 65); GGSRLVCWVPFEDFMDSICTEYGG (18F01; SEQ ID NO: 66); or GGDTYTCFFPWPGLDDHFCTYSGG (17D12; SEQ ID NO: 67); or (ii) an amino acid sequence having 1, 2, 3, or 4 substitution(s), addition(s), and/or deletion(s) relative to the amino acid sequence of (i). In another embodiment, X261, X262, X283, and X284 are absent and examples of the IL-18R alpha binding moiety include, but are not limited to, (i) the amino acid sequence of HDLLCFRPWEDVEDLVCTED (18C04-G; SEQ ID NO: 68); ERLMCSVPWEDVADFICMGD (18F04-G; SEQ ID NO: 69); VQMICWLPWPDVDDHICTEV (17G07-G; SEQ ID NO: 70); NHRICWLPWEDVDDRICISR (17C07-G; SEQ ID NO: 71); LYSMCFRPWEDVEDFICTTN (17F07-G; SEQ ID NO: 72); RSTLCWVPWEGLGDRICTAQ (17F12-G; SEQ ID NO: 73); SYLTCWVPWQGLDDRFCTET (17E09-G; SEQ ID NO: 74); TDYTCFVPWNDVFDRVCTWS (18D03-G; SEQ ID NO: 75); SHLICWMPWPEVEDRLCTDS (17C08-G; SEQ ID NO: 76); TKLMCWVPWQDVDDFICTSL (18C03-G; SEQ ID NO: 77); FYQSCWHPWEDIDDHICTYS (18B03-G; SEQ ID NO: 78); FKLSCWVPWQDTEDRICWTV (18E02-G; SEQ ID NO: 79); SHWICWWPFSDTEDHICSEY (17A09-G; SEQ ID NO: 80); SRLVCWVPFEDFMDSICTEY (18F01-G; SEQ ID NO: 81); or DTYTCFFPWPGLDDHFCTYS (17D12-G; SEQ ID NO: 82); or (ii) an amino acid sequence having 1, 2, 3, or 4 substitution(s), addition(s), and/or deletion(s) relative to the amino acid sequence of (i).

### IL-18R Alpha Sequence Family 2

In another aspect, an IL-18R alpha binding moiety can have the amino acid sequence of X21-X22-X23-X24-X25-X26-X27-X28-X29-X30-X31-X32-X33-X34-X35-X36-X37-X38-X39-X40; wherein: (i) one or more of X21, X22, X39, and X40 are optional; (ii) if present, X21 is G; if present, X22 is G; X23 if F or S; X24 is N or W; X25 is V, A, or L; X26 is C; X27 is D or S; X28 is F or L; X29 is D or E; X30 is P; X31 is G, X32 is W; X33 is W; X34 is D, G, or E; X35 is C; X36 is Xaa; X37 is Xaa; X38 is Xaa; if present, X39 is G; and if present, X40 is G; and (iii) Xaa is any amino acid (SEQ ID NO: 457). In one embodiment, X23 is F; X24 is N; X25 is I, L, M, V, F, Y, W, A, G, P, S, or T; X26 is C; X27 is S; X28 is I, L, M, V, F, Y, W, A, G, P, S, or T; X29 is D or E; X30 is P; X31 is G, X32 is W; X33 is W; X34 is D or E; X35 is C; X36 is Xaa; X37 is Xaa; X38 is Xaa (alpha family 2 consensus; SEQ ID NO: 458). In another embodiment, the IL-18R alpha binding moiety has (i) the amino acid sequence of, for example, GGFNVCDFDPGWWDCLQLGG (2E11; SEQ ID NO: 83); GGFNACSFEPGWWGCEVYGG (4B03; SEQ ID NO: 84); or GGSWLCSLEPGWWECNPGGG (4C03; SEQ ID NO: 85), or (ii) an amino acid sequence having 1, 2, 3, or 4 substitution(s), addition(s), and/or deletion(s) relative to an amino acid sequence of (i). In another embodiment, X21, X22, X39, and X40 are absent and examples of the IL-18R alpha binding moiety include, but are not limited to, (i) the amino acid sequence of FNVCDFDPGWWDCLQL (2E11-G; SEQ ID NO: 86); FNACSFEPGWWGCEVY (4B03-G; SEQ ID NO: 87); or SWLCSLEPGWWECNPG (4C03-G; SEQ ID NO: 88), or (ii) an amino acid sequence having 1, 2, 3, or 4 substitution(s), addition(s), and/or deletion(s) relative to an amino acid sequence of any one of (i).

### IL-18R Alpha Sequence Family 3

In another aspect, an IL-18R alpha binding moiety can have the amino acid sequence of X41-X42-X43-X44-X45-X46-X47-X48-X49-X50-X51-X52-X53-X54-X55-X56-X57-X58-X59-X60; wherein: (i) one or more of X41, X42, X43, X44, X58, X59, and X60 are optional, (ii) if present, X41 is G; if present, X42 is G; if present, X43 is Xaa; X44 is L, C, or G; X45 is Xaa; X46 is C, L, or S; X47 is D, V, E, or R; X48 is Y or W; X49 is I, L, M, V, F, Y, W, A, G, P, S, or T; X50 is P, X51 is G or F; X52 is Xaa; X53 is W, H, S, F, or Y; X54 is I, M, F, Y, W, A, G, P, S, T, L or V; X55 is C, E, or V; X56 is Xaa; X57 is E, C, or D; if present, X58 is Xaa; if present, X59 is G; and if present, X60 is G; and (iii) Xaa is any amino acid (SEQ ID NO: 484). In one embodiment, X43 is Xaa; X44 is C; X45 is Xaa; X46 is L; X47 is E or D; X48 is W; X49 is I, L, M, V, F, Y, W, A, G, P, S, or T; X50 is P; X51 is F; X52 is Xaa; X53 is F, H, W, or Y; X54 is I, L, M, V, F, Y, W, A, G, P, S, or T; X55 is E; X56 is Xaa; X57 is E, C, or D; and X58 is Xaa (alpha family 3 consensus; SEQ ID NO: 459). In another embodiment, the IL-18R alpha binding moiety has (i) the amino acid sequence of, for example, GGSLVCDYLPGMSLCYESGG (4G04; SEQ ID NO: 89) or (ii) an amino acid sequence having 1, 2, 3, or 4 substitution(s), addition(s), and/or deletion(s) relative to the amino acid sequence of (i). In another embodiment, X41, X42, X59, and X60 are absent and examples of the IL-18R alpha binding moiety include, but are not limited to, (i) the amino acid sequence of SLVCDYLPGMSLCYES (4G04-G; SEQ ID NO: 90) or (ii) an amino acid sequence having 1, 2, 3, or 4 substitution(s), addition(s), and/or deletion(s) relative to the amino acid sequence of (i). In another embodiment, X41 and X60 are absent and examples of the IL-18R alpha binding moiety include, but are not limited to, (i) the amino acid sequence of GGCELVWTPFQFVEHCGG (1F04; SEQ ID NO: 91) or GGCSLEWVPFTYVEECGG (1E11; SEQ ID NO: 92); or (ii) an amino acid sequence having 1, 2, 3, or 4 substitution(s), addition(s), and/or deletion(s) relative to an amino acid sequence of any one of (i). In another embodiment, X41, X42, X43, X58, X59, and X60 are absent and examples of the IL-18R alpha binding moiety include, but are not limited to, (i) the amino acid sequence of CELVWTPFQFVEHC (1F04-G; SEQ ID NO: 93) or CSLEWVPFTYVEEC (1E11-G; SEQ ID NO: 94); or (ii) an amino acid sequence having 1, 2, 3, or 4 substitution(s), addition(s), and/or deletion(s) relative to an amino acid sequence of any one of (i). In another embodiment, X41 and X42 are absent and examples of the IL-18R alpha binding moiety include, but are not limited to, (i) the amino acid sequence of GGCSRWVPFHYVVGDCGG (1D07; SEQ ID NO: 95) or (ii) an amino acid sequence having 1, 2, 3, or 4 amino acid substitution(s), addition(s), and/or deletion(s) relative to the amino acid sequence of (i). In another embodiment, X41, X42, X43, X44, X59, and X60 are absent and examples of the IL-18R alpha binding moiety include, but are not limited to, (i) the amino acid sequence of CSRWVPFHYVVGDC (1D07-G; SEQ ID NO: 96) or (ii) an amino acid sequence having 1, 2, 3, or 4 amino acid substitution(s), addition(s), and/or deletion(s) relative to the amino acid sequence of (i).

### IL-18R alpha Sequence Family 4

In another aspect, an IL-18R alpha binding moiety can have the amino acid sequence of X61-X62-X63-X64-X65-X66-X67-X68-X69-X70-X71-X72-X73-X74-X75-X76-X77-X78-X79-X80-X81-X82-X83; wherein: (i) one or more of X61, X62, X63, X64, X65, X80, X81, X82, X83, and X84 are optional, (ii) if present, X61 is G; if present, X62 is G; if present, X63 is Xaa; if present, X64 is G, N, S, V, D, Y, E, or A; if present, X65 is M, A, P, T, W, F, G, Y, F, S, V, H, D, K, L, or I; X66 is I, V, Y, A, G, P, T, S, F, M, L, or W; X67 is Q, E, or D; X68 is C; X69 is L, F, W, A, or Y; X70 is W or Y; X71 is E or S; X72 is P, X73 is L, G, H, T, S, Y, or T; X74 is W, H, E, Q, or A; X75 is E, V, Y, H, or L; X76 is C or W; X77 is W or C; X78 is T, I, S, M, or W; X79 is D, P, or T; if present, X80 is G, S, H, I, T, or A; if present, X81 is M, W, A, P, S, T, G, L, V, F, Y, Q, or I; if present, X82 is V, F, Y, A, S, G, M, I, T, N, F, D, L, Q, W, or P; if present, X83 is G; and if present, X84 is G; and (iii) Xaa is any amino acid (SEQ ID NO: 485). In one embodiment, X63 is Xaa; X64 is N, D, or E; X65 is I, L, M, V, F, Y, W, A, G, P, S, T, H, or Y; X66 is I, L, M, V, F, Y, W, A, G, P, S, or T; X67 is E, or D; X68 is C; X69 is F; X70 is W or Y; X71 is S; X72 is P, X73 is G or S; X74 is Q; X75 is E; X76 is W; X77 is C; X78 is I; X79 is P; X80 is S; X81 is I, L, M, V, F, Y, W, A, G, P, S, or T; X82 is I, L, M, V, F, Y, W, A, G, P, S, or T; wherein Xaa is any amino acid (SEQ ID NO: 460). In another embodiment, the IL-18R alpha binding moiety has (i) the amino acid sequence of, for example, GGANYLECFYSPTQEWCIPHLMGG (10G04; SEQ ID NO: 97); GGESFLECFYSPAEWCIPSVIGG (10G07; SEQ ID NO: 98); GGFVFMECFWSPSQEWCIPSFTGG (10B07; SEQ ID NO: 99); GGGVSFDCFWSPGQYWCIPDYNGG (10F07; SEQ ID NO: 100); GGLDVMECFWSPSQVWCMPSVFGG (10E06; SEQ ID NO: 101); GGLYHFECAWSPYQHWCWPSQDGG (10B12; SEQ ID NO: 102); GGREDMECFWSPGQVWCIPSYLGG (10D09; SEQ ID NO: 103); GGREKLECFWSPGQEWCIPILQGG (10001; SEQ ID NO: 104); GGRGFLECFYSPGQEWCIPSIWGG (10H01; SEQ ID NO: 105); GGSALLECFYSPTQEWCIPTLWGG (10007; SEQ ID NO: 106); GGSSYSECFWSPGQLWCIPSFPGG (10E02; SEQ ID NO: 107); GGVSIFECFYSPTQEWCIPSFIGG (10B02; SEQ ID NO: 108); GGWDILECFYSPGQEWCIPSFLGG (10H03; SEQ ID NO: 109); or GGYGSWECYWSPSEEWCIPAQLGG (100O2; SEQ ID NO: 110); or (ii) an amino acid sequence having 1, 2, 3, or 4 substitution(s), addition(s), and/or deletion(s) relative to an amino acid sequence of any one of (i). In another embodiment, X61, X62, X83, and X84 are absent and examples of the IL-18R alpha binding moiety include, but are not limited to, (i) the amino acid sequence of ANYLECFYSPTQEWCIPHLM (10G04-G; SEQ ID NO: 111); ESFLECFYSPAEWCIPSVI (10G07-G; SEQ ID NO: 112); FVFMECFWSPSQEWCIPSFT (10B07-G; SEQ ID NO: 113); GVSFDCFWSPGQYWCIPDYN (10F07-G; SEQ ID NO: 114); LDVMECFWSPSQVWCMPSVF (10E06-G; SEQ ID NO: 115); LYHFECAWSPYQHWCWPSQD (10B12-G; SEQ ID NO: 116); REDMECFWSPGQVWCIPSYL (10D09-G; SEQ ID NO: 117); REKLECFWSPGQEWCIPILQ (10001-G; SEQ ID NO: 118); RGFLECFYSPGQEWCIPSIW (10H01-G; SEQ ID NO: 119); SALLECFYSPTQEWCIPTLW (10007-G; SEQ ID NO: 120); SSYSECFWSPGQLWCIPSFP (10E02-G; SEQ ID NO: 121); VSIFECFYSPTQEWCIPSFI (10B02-G; SEQ ID NO: 122); WDILECFYSPGQEWCIPSFL (10H03-G; SEQ ID NO: 123); or YGSWECYWSPSEEWCIPAQL (100O2-G; SEQ ID NO: 124), or (ii) an amino acid sequence having 1, 2, 3, or 4 substitution(s), addition(s), and/or deletion(s) relative to an amino acid sequence of any one of (i). In another embodiment, X61, X62, X82, X83, and X84 are absent and examples of the IL-18R alpha binding moiety include, but are not limited to, (i) the amino acid sequence of GGWSQCLWEPLWECWTDGG (7C02; SEQ ID NO: 125) or (ii) an amino acid sequence having 1, 2, 3, or 4 substitution(s), addition(s), and/or deletion(s) relative to the amino acid sequence of (i). In another embodiment, X61, X62, X63, X64, X80, X81, X82, X83, and X84 are absent and examples of the IL-18R alpha binding moiety include, but are not limited to, (i) the amino acid sequence of GGWSQCLWEPLWECWTDGG (7C02-G; SEQ ID NO: 125) or (ii) an amino acid sequence having 1, 2, 3, or 4 substitution(s), addition(s), and/or deletion(s) relative to the amino acid sequence of (i). In another embodiment, X61, X62, X83, and X84 are absent and examples of the IL-18R alpha binding moiety include, but are not limited to, (i) the amino acid sequence of GGFFECFYSPGHEWCIPSGG (7C06; SEQ ID NO: 126) or (ii) an amino acid sequence having 1, 2, 3, or 4 substitution(s), addition(s), and/or deletion(s) relative to the amino acid sequence of (i). In another embodiment, X61, X62, X63, X64, X81, X82, X83, and X84 are absent and examples of the IL-18R alpha binding moiety include, but are not limited to, (i) the amino acid sequence of FFECFYSPGHEWCIPS (7C06-G; SEQ ID NO: 127) or (ii) an amino acid sequence having 1, 2, 3, or 4 substitution(s), addition(s), and/or deletion(s) relative to the amino acid sequence of (i). In another embodiment, X61, X62, X63, X82, X83, and X84 are absent and examples of the IL-18R alpha binding moiety include, but are not limited to, (i) the amino acid sequence of GGMECWYSPHEVWCSTGG (6G11; SEQ ID NO: 128) or (ii) an amino acid sequence having 1, 2, 3, or 4 substitution(s), addition(s), and/or deletion(s) relative to the amino acid sequence of (i). In another embodiment, X61, X62, X63, X64, X65, X80, X81, X82, X83, and X84 are absent and examples of the IL-18R alpha binding moiety include, but are not limited to, (i) the amino acid sequence of MECWYSPHEVWCST (6G11-G; SEQ ID NO: 129) or (ii) an amino acid sequence having 1, 2, 3, or 4 substitution(s), addition(s), and/or deletion(s) relative to the amino acid sequence of (i).

### IL-18R Alpha Sequence Family 5

In another aspect, an IL-18R alpha binding moiety can have the amino acid sequence of X85-X86-X87-X88-X89-X90-X91-X92-X93-X94-X95-X96-X97-X98-X99-X100-X101-X102-X103-X104; wherein: (i) one or more of X85, X86, X102, and X104 are optional, (ii) if present, X85 is G; if present, X86 is G; X87 is Xaa, or F; X88 is Xaa, or X; X89 is C; X90 is E, Y, or W; X91 is S, G, P, or T; X92 is M, D, or Q; X93 is E, S, N, or P; X94 is P; X95 is G; X96 is Q, D, or I; X97 is Y or D; X98 is T, Y, W, or K; X99 is E, V, or N; X100 is C; X101 is Xaa; X102 is F, H, W, E, P, V, or Y; if present, X103 is G; and if present, X104 is G, and (iii) Xaa is any amino acid (SEQ ID NO: 461). In one embodiment, X87 is Xaa; X88 is Xaa; X89 is C; X90 is Y, or W; X91 is P; X92 is D or Q; X93 is E or N; X94 is P; X95 is G; X96 is I; X97 is Y; X98 is Y or W; X99 is E or N; X100 is C; X101 is Xaa; X102 is F, H, W, or Y (alpha family 5 consensus; SEQ ID NO: 462). In another embodiment, the IL-18R alpha binding moiety has (i) the amino acid sequence of, for example, GGPYCESMEPGQYTECTWGG (6C03; SEQ ID NO: 130); GGLVCYGDSPGDDYVCIEGG (6A04; SEQ ID NO: 131); GGWECWPDNPGIYWNCQPGG (6C05; SEQ ID NO: 132); GGRNCYTMPPGIYKECAWGG (6F03; SEQ ID NO: 133); GGFSCWPQEPGIYYNCLVGG (6D04; SEQ ID NO: 134); or GGPSCWPQEPGIYYNCIYGG (6C02; SEQ ID NO: 135); or (ii) an amino acid sequence having 1, 2, 3, or 4 substitution(s), addition(s), and/or deletion(s) relative to an amino acid sequence of any one of (i). In another embodiment, X85, X86, X103, and X104 are absent and examples of the IL-18R alpha binding moiety include, but are not limited to, (i) the amino acid sequence of PYCESMEPGQYTECTW (6C03-G; SEQ ID NO: 136); LVCYGDSPGDDYVCIE (6A04-G; SEQ ID NO: 137); WECWPDNPGIYWNCQP (6C05-G; SEQ ID NO: 138); RNCYTMPPGIYKECAW (6F03-G; SEQ ID NO: 139); FSCWPQEPGIYYNCLV (6D04-G; SEQ ID NO: 140); or PSCWPQEPGIYYNCIY (6C02-G; SEQ ID NO: 141); or (ii) an amino acid sequence having 1, 2, 3, or 4 substitution(s), addition(s), and/or deletion(s) relative to an amino acid sequence of any one of (i).

### IL-18Ra Sequence Family 6

In another aspect, an IL-18R alpha binding moiety can have (i) the amino acid sequence of, for example, GGCNYKEYQDGIWMVCGG (1E06; SEQ ID NO: 142); GGVFTCPPPPGENHCGVFGG (2G09; SEQ ID NO: 143); GGDCWDQNTFRYIDCFGG (1E03; SEQ ID NO: 144); GGRSDGFVWWLGWLGG (1C04; SEQ ID NO: 145); GGMFEAEWFLEQFGGG (3H06; SEQ ID NO: 146); GGYRFLCQDGFCLQWSGG (4D11; SEQ ID NO: 147); GGASFCDILPGMEWCTYDGG (7C04; SEQ ID NO: 148); GGQACTRNFWGLYRCNAGG (6Al2; SEQ ID NO: 149); GGLICDMQDGMSFCYDGG (12E05; SEQ ID NO: 150); or GGCYWIEKESTWHVECGG (12F01; SEQ ID NO: 151); or (ii) an amino acid sequence having 1, 2, 3, or 4 substitution(s), addition(s), and/or deletion(s) relative to an amino acid sequence of any one of (i). Alternatively, an IL-18R alpha binding moiety can have (i) the amino acid sequence of, for example, CNYKEYQDGIWMVC (1E06-G; SEQ ID NO: 152); VFTCPPPPGENHCGVF (2G09-G; SEQ ID NO: 153); DCWDQNTFRYIDCF (1E03-G; SEQ ID NO: 154); RSDGFVWWLGWL (1C04-G; SEQ ID NO: 155); MFEAEWFLEQFG (3H06-G; SEQ ID NO: 156); YRFLCQDGFCLQWS (4D11-G; SEQ ID NO: 157); ASFCDILPGMEWCTYD (7C04-G; SEQ ID NO: 158); QACTRNFWGLYRCNA (6A12-G; SEQ ID NO: 159); LICDMQDGMSFCYD (12E05-G; SEQ ID NO: 160); or CYWIEKESTWHVEC (12F01-G; SEQ ID NO: 161); or (ii) an amino acid sequence having 1, 2, 3, or 4 substitution(s), addition(s), and/or deletion(s) relative to an amino acid sequence of any one of (i).

In another aspect, an IL-18R alpha binding moiety can have (i) the amino acid sequence of, for example, GGREDMECFWSPGQVWCIPSYLGG (10D09 trunc 01; SEQ ID NO: 103); REDMECFWSPGQVWCIPSYLGG (10D09 trunc 02; SEQ ID NO: 162); EDMECFWSPGQVWCIPSYLGG (10D09 trunc 03; SEQ ID NO: 163); DMECFWSPGQVWCIPSYLGG (10D09 trunc 04; SEQ ID NO: 164); MECFWSPGQVWCIPSYLGG (10D09 trunc 05; SEQ ID NO: 165); ECFWSPGQVWCIPSYLGG (10D09 trunc 06; SEQ ID NO: 166); GGREDMECFWSPGQVWCIPSYL (10D09 trunc 07; SEQ ID NO: 167); GGREDMECFWSPGQVWCIPSY (10D09 trunc 08; SEQ ID NO: 168); or GGREDMECFWSPGQVWCIPS (10D09 trunc 09; SEQ ID NO: 169); or (ii) an amino acid sequence having 1, 2, 3, or 4 substitution(s), addition(s), and/or deletion(s) relative to an amino acid sequence of any one of (i). Alternatively, an IL-18R alpha binding moiety can have (i) the amino acid sequence of, for example, REDMECFWSPGQVWCIPSYL (10D09 trunc 01-G; SEQ ID NO: 117); REDMECFWSPGQVWCIPSYL (10D09 trunc 02-G; SEQ ID NO: 117); EDMECFWSPGQVWCIPSYL (10D09 trunc 03-G; SEQ ID NO: 170); DMECFWSPGQVWCIPSYL (10D09 trunc 04-G; SEQ ID NO: 171); MECFWSPGQVWCIPSYL (10D09 trunc 05-G; SEQ ID NO: 172); ECFWSPGQVWCIPSYL (10D09 trunc 06-G; SEQ ID NO: 173); REDMECFWSPGQVWCIPSYL (10D09 trunc 07-G; SEQ ID NO: 117); REDMECFWSPGQVWCIPSY (10D09 trunc 08-G; SEQ ID NO: 174); or REDMECFWSPGQVWCIPS (10D09 trunc 09-G; SEQ ID NO: 175); or (ii) an amino acid sequence having 1, 2, 3, or 4 substitution(s), addition(s), and/or deletion(s) relative to an amino acid sequence of any one of (i).

### IL-18R Alpha Sequence Family 7

In another aspect, an IL-18R alpha binding moiety can have the amino acid sequence of X105-X106-X107-X108-X109-X110-X111-X112-X113-X114-X115-X116-X117-X118-X119-X120-X121-X122-X123-X124-X125-X126-X127-X128, wherein: (i) one or more of X105, X106, X107, X108, X123, X124, X125, X126, X127, and X128 are optional, (ii) if present, X105 is G; if present, X106 is G; if present, X107 is G or Q; if present, X108 is Xaa; X109 is Xaa; X110 is L or C; X111 is H, F, or W; X112 is C, W, Q, or I; X113 is F, V, D, or S; X114 is E, N, Y, D, or L; X115 is S, D, or P; X116 is R, M, or G; X117 is Xaa; X118 is Xaa; X119 is M, F, Y, W, A, G, P, S, T, D, V, L, or I; X120 is I, L, M, F, Y, A, G, P, S, T, F, H, W, V, or Y; X121 is C; X122 is Xaa; if present, X123 is Xaa; if present, X124 is Xaa; if present, X125 is Xaa; if present, X126 is S; if present, X127 is G; and if present, X128 is G, and (iii) Xaa is any amino acid (SEQ ID NO: 486). In one embodiment, X108 is Xaa; X109 is Xaa; X110 is C; X111 is W; X112 is Q; X113 is D; X114 is E, N, or D; X115 is S or P; X116 is G; X117 is Xaa; X118 is Xaa; X119 is I, L, M, V, F, Y, W, A, G, P, S, or T; X120 is I, L, M, V, F, Y, W, A, G, P, S, T, or H; X121 is C; X122 is Xaa; X123 is Xaa; X124 is Xaa; and X125 is Xaa (consensus sequence; SEQ ID NO: 463). Examples of the IL-18R alpha binding moiety include, but are not limited to, (i) the amino acid sequence of GGQTFLHCFESRGIDWCIPWKSGG (11G01; SEQ ID NO: 176) or (ii) an amino acid sequence having 1, 2, 3, or 4 substitution(s), addition(s), and/or deletion(s) relative to the amino acid sequence of (i). In another embodiment, X105, X106, X127, and X128 are absent and examples of the IL-18R alpha binding moiety include, but are not limited to, (i) the amino acid sequence of QTFLHCFESRGIDWCIPWKS (11G01-G; SEQ ID NO: 177) or (ii) an amino acid sequence having 1, 2, 3, or 4 substitution(s), addition(s), and/or deletion(s) relative to the amino acid sequence of (i). In another embodiment, X105, X106, X125, X126, X127, and X128 are absent and examples of the IL-18R alpha binding moiety include, but are not limited to, (i) the amino acid sequence of GGHCFWVNDMMEVVCDGG (12B04; SEQ ID NO: 178) or (ii) an amino acid sequence having 1, 2, 3, or 4 substitution(s), addition(s), and/or deletion(s) relative to the amino acid sequence of (i). In another embodiment, X105, X106, X107 X108, X123, X124, X125, X126, X127, and X128 are absent and examples of the IL-18R alpha binding moiety include, but are not limited to, (i) the amino acid sequence of HCFWVNDMMEVVCD (12B04-G; SEQ ID NO: 179) or (ii) an amino acid sequence having 1, 2, 3, or 4 substitution(s), addition(s), and/or deletion(s) relative to the amino acid sequence of (i). In another embodiment, X105, X126, X127, and X128 are absent and examples of the IL-18R alpha binding moiety include, but are not limited to, (i) the amino acid sequence of GGFTCWQDYPGDTVYCYPGG (12D08; SEQ ID NO: 180); GGQECWISDSGTYLWCTQGG (12D10; SEQ ID NO: 181); or GGWECWQDLPGSSIVCTDGG (12G10; SEQ ID NO: 182); or (ii) an amino acid sequence having 1, 2, 3, or 4 substitution(s), addition(s), and/or deletion(s) relative to an amino acid sequence of any one of (i). In another embodiment, X105, X106, X107, X124, X125, X126, X127, and X128 are absent and examples of the IL-18R alpha binding moiety include, but are not limited to, (i) the amino acid sequence of FTCWQDYPGDTVYCYP (12D08-G; SEQ ID NO: 183); QECWISDSGTYLWCTQ (12D10-G; SEQ ID NO: 184); or WECWQDLPGSSIVCTD (12G10-G; SEQ ID NO: 185); or (ii) an amino acid sequence having 1, 2, 3, or 4 substitution(s), addition(s), and/or deletion(s) relative to an amino acid sequence of any one of (i).

### IL-18Ra Sequence Family 8

In another aspect, an IL-18R alpha binding moiety can have the amino acid sequence of X129-X130-X131-X132-X133-X134-X135-X136-X137-X138-X139-X140-X141-X142-X143-X144-X145-X146-X147-X148; wherein: (i) one or more of X129, X130, X145, X146, X147, and X148 are optional; (ii) if present, X129 is G; if present, X130 is G; X131 is Xaa; X132 is N, C, F, D, or S; X133 is D, R, L, or C; X134 is I, M, F, W, A, G, P, Y, T, S, E, V, L, or W; X135 is F, F, or P; X136 is N, L, or T; X137 is R, S, P, or W; X138 is L, R, or P; X139 is W, L, V, or G; X140 is E, S, W, D, or E; X141 is L, F, M, N, T, E, or A; X142 is P, I, F, T, S, I, or D; X143 is D, H, C, P, S, or W; X144 is C or W; if present, X145 is Xaa; if present, X146 is Xaa; if present, X147 is G; and if present, X148 is G, and (iii) Xaa is any amino acid (SEQ ID NO: 487). In one embodiment, X131 is Xaa; X132 is D or N; X133 is C; X134 is I, L, M, V, F, Y, W, A, G, P, S, or T; X135 is P; X136 is L; X137 is W; X138 is P; X139 is G; X140 is D or E; X141 is A; X142 is D; X143 is P or W; X144 is C; X145 is Xaa; and X146 is Xaa, wherein Xaa is any amino acid (consensus; SEQ ID NO: 464). In another embodiment, the IL-18R alpha binding moiety has (i) the amino acid sequence of, for example, GGDFCEPLWPGDNTPCIVGG (12E11; SEQ ID NO: 186); GGHNCVPLWPGDTSPCYDGG (12H10; SEQ ID NO: 187); GGQDCYPLWPGEEISCETGG (12B08; SEQ ID NO: 188); GGLDCLPLWPGDADWCLAGG (12F11; SEQ ID NO: 189); or GGDSCWVLWPGDADWCTIGG (12B11; SEQ ID NO: 190); or (ii) an amino acid sequence having 1, 2, 3, or 4 substitution(s), addition(s), and/or deletion(s) relative to an amino acid sequence of any one of (i). In another embodiment, X129, X130, X147, and X148 are absent and examples of the IL-18R alpha binding moiety include, but are not limited to, (i) the amino acid sequence of DFCEPLWPGDNTPCIV (12E11-G; SEQ ID NO: 191); HNCVPLWPGDTSPCYD (12H10-G; SEQ ID NO: 192); QDCYPLWPGEEISCET (12B08-G; SEQ ID NO: 193); LDCLPLWPGDADWCLA (12F11-G; SEQ ID NO: 194); or DSCWVLWPGDADWCTI (12B11-G; SEQ ID NO: 195); or (ii) an amino acid sequence having 1, 2, 3, or 4 substitution(s), addition(s), and/or deletion(s) relative to an amino acid sequence of any one of (i). In another embodiment, X148 is absent and examples of the IL-18R alpha binding moiety include, but are not limited to, (i) the amino acid sequence of GGFCRTFLSLLSFIHCHGG (13G03; SEQ ID NO: 196) or (ii) an amino acid sequence having 1, 2, 3, or 4 substitution(s), addition(s), and/or deletion(s) relative to the amino acid sequence of (i). In another embodiment, X129, X130, X146, X147, and X148 are absent and examples of the IL-18R alpha binding moiety include, but are not limited to, (i) the amino acid sequence of FCRTFLSLLSFIHCH (13G03-G; SEQ ID NO: 197) or (ii) an amino acid sequence having 1, 2, 3, or 4 substitution(s), addition(s), and/or deletion(s) relative to the amino acid sequence of (i). In another embodiment, wherein X147 and X148 are absent and examples of the IL-18R alpha binding moiety include, but are not limited to, (i) the amino acid sequence of GGCNDYVNRLWELPDCGG (12A01; SEQ ID NO: 198) or GGGCLSFTPRVWMFCWGG (13A09; SEQ ID NO: 199) or (ii) an amino acid sequence having 1, 2, 3, or 4 substitution(s), addition(s), and/or deletion(s) relative to an amino acid sequence of any one of (i). In another embodiment, X129, X130, X145, X146, X147 and X148 are absent and examples of the IL-18R alpha binding moiety include, but are not limited to, (i) the amino acid sequence of CNDYVNRLWELPDC (12A01-G; SEQ ID NO: 200) or GGGCLSFTPRVWMFCWGG (13A09-G; SEQ ID NO: 199); or (ii) an amino acid sequence having 1, 2, 3, or 4 substitution(s), addition(s), and/or deletion(s) relative to an amino acid sequence of any one of (i).

### IL-18R Beta Binding Moieties

In another aspect, provided herein is an IL-18R binding moiety comprising an IL-18R beta binding moiety. An IL-18R beta binding moiety can, in some instances, be an IL-18R beta agonist. Alternatively, an IL-18R beta binding moiety can, in some instances, be an IL-18R beta antagonist. Each of the IL-18R beta binding moieties were found to bind to IL-18R beta with a binding affinity (K_{D} or IC50) of less than 10 μM (data not shown). The IL-18R beta binding moiety either (a) does not bind to the IL-18BP or (b) binds to the IL-18BP with a K_{D} that is at least 1,000-fold higher than the K_{D} of the interaction between the IL-18R beta binding moiety and IL-18R beta. In one instance, the IL-18R beta binding moiety does not bind to the IL-18BP. In another instance, the IL-18R beta binding moiety binds to the IL-18BP with a K_{D} that is at least 1,000-fold higher than the K_{D} of the interaction between the IL-18R beta binding moiety and IL-18R beta.

### IL-18R Beta Sequence Family 1

In one aspect, an IL-18R beta binding moiety can have the amino acid sequence of X149-X150-X151-X152-X153-X154-X155-X156-X157-X158-X159-X160-X161-X162-X163-X164-X165-X166-X167-X168; wherein: (i) one or more of X149, X150, X151, X166, X167, and X168 are optional, (ii) if present, X149 is G; if present, X150 is G; if present, X151 is G or R; X152 is Y, K, F, or P; X153 is T, V, F, H, P, C, Y, I, L, M, W, A, G, or S; X154 is S, C, or Q; X155 is I, Y, W, G, P, T, F, M, D, A, H, V, L, or S; X156 is H, N, Y, F, W, L, or G; X157 is D, V, I, R, E, or G; X158 is Xaa; X159 is Xaa; X160 is I, F, Y, A, G, P, E, N, S, W, M, V, T, or L; X161 is Xaa; X162 is M, Y, W, G, S, T, L, I, V, E, A, F, or P; X163 is C, D, or Y; X164 is V, F, Y, W, A, G, P, T, L, M, I, S, R, or C; X165 is F, Y, W, A, G, P, S, T, V, I, L, M, or R; if present, X166 is G, R, or L; if present X167 is G; and if present, X168 is G (SEQ ID NO: 488); and (iii) Xaa is any amino acid. In one embodiment, X152 is F or Y; X153 is I, L, M, V, F, Y, W, A, G, P, S, or T; X154 is C; X155 is I, L, M, V, F, Y, W, A, G, P, S, or T; X156 is F, H, W, or Y; X157 is D or E; X158 is Xaa, X159 is Xaa, X160 is I, L, M, V, F, Y, W, A, G, P, S, or T; X161 is Xaa; X162 is I, L, M, V, F, Y, W, A, G, P, S, or T; X163 is C; X164 is I, L, M, V, F, Y, W, A, G, P, S, or T; and X165 is I, L, M, V, F, Y, W, A, G, P, S, or T (beta family 1 consensus; SEQ ID NO: 489). In another embodiment, X168 is absent and examples of the IL-18R beta binding moiety include, but are not limited to, (i) the amino acid sequence of GGRFCQVLDNGVHACLIGG (3H05; SEQ ID NO: 201) or (ii) an amino acid sequence having 1, 2, 3, or 4 substitution(s), addition(s), and/or deletion(s) relative to the amino acid sequence of (i). In another embodiment, X149, X150, X166, X167, and X168 are absent and examples of the IL-18R beta binding moiety include, but are not limited to, (i) the amino acid sequence of RFCQVLDNGVHACLI (3H05-G; SEQ ID NO: 202) or (ii) an amino acid sequence having 1, 2, 3, or 4 substitution(s), addition(s), and/or deletion(s) relative to the amino acid sequence of (i). In another embodiment, X149 is absent and examples of the IL-18R beta binding moiety include, but are not limited to, (i) the amino acid sequence of GGPYCLWGSGTYFDCMRGG (3D08; SEQ ID NO: 203) or GGKFCSFGAMSLPYCRLGG (3A04; SEQ ID NO: 204); or (ii) an amino acid sequence having 1, 2, 3, or 4 substitution(s), addition(s), and/or deletion(s) relative to an amino acid sequence of any one of (i). In another embodiment, X149, X150, X151, X167, and X168 are absent and examples of the IL-18R beta binding moiety include, but are not limited to, (i) the amino acid sequence of PYCLWGSGTYFDCMR (3D08-G; SEQ ID NO: 205) or KFCSFGAMSLPYCRL (3A04-G; SEQ ID NO: 206); or (ii) an amino acid sequence having 1, 2, 3, or 4 substitution(s), addition(s), and/or deletion(s) relative to an amino acid sequence of any one of (i). In another embodiment, X149 and X168 are absent and examples of the IL-18R beta binding moiety include, but are not limited to, (i) the amino acid sequence of GGYTCMNDHPEILCLTGG (2A01; SEQ ID NO: 207); GGKVCDYVTSNGICMVGG (2G04; SEQ ID NO: 208); GGYHCDWIDESGVCIVGG (2E02; SEQ ID NO: 209); GGFPCAFRAPWAECSIGG (3B05; SEQ ID NO: 210); or GGKVCHWEGQMLLCRLGG (3D10; SEQ ID NO: 211); or (ii) an amino acid sequence having 1, 2, 3, or 4 substitution(s), addition(s), and/or deletion(s) relative to an amino acid sequence of any one of (i). In another embodiment, X149, X150, X151, X166, X167, and X168 are absent and examples of the IL-18R beta binding moiety include, but are not limited to, (i) the amino acid sequence of YTCMNDHPEILCLT (2A01-G; SEQ ID NO: 212); KVCDYVTSNGICMV (2G04-G; SEQ ID NO: 213); YHCDWIDESGVCIV (2E02-G; SEQ ID NO: 214); FPCAFRAPWAECSI (3B05-G; SEQ ID NO: 215); or KVCHWEGQMLLCRL (3D10-G; SEQ ID NO: 216); or (ii) an amino acid sequence having 1, 2, 3, or 4 substitution(s), addition(s), and/or deletion(s) relative to an amino acid sequence of any one of (i). In another embodiment, X150 is G; X151 is G; X152 is Y; X153 is V; X154 is C; X155 is L; X156 is W; X157 is D; X158 is A; X159 is Q; X160 is V; X161 is A; X162 is L; X163 is C; X164 is L; X165 is V; X166 is G; and X167 is G (beta synthetic construct 1; SEQ ID NO: 465). In another embodiment, X150 is G; X151 is G; X152 is F; X153 is C; X154 is S; X155 is F; X156 is G; X157 is D; X158 is G; X159 is T; X160 is L; X161 is P; X162 is A; X163 is C; X164 is L; X165 is I; X166 is G; and X167 is G (beta synthetic construct 2; SEQ ID NO: 466).

### IL-18R Beta Sequence Family 2

In another aspect, an IL-18R beta binding moiety can have the amino acid sequence of X169-X170-X171-X172-X173-X174-X175-X176-X177-X178-X179-X180-X181-X182-X183-X184-X185-X186-X187-X188-X189-X190-X191-X192; wherein: (i) one or more of X169, X170, X171 X172 X173, X187, X188, X189, X190, X191, and X192 are optional, (ii) if present, X169 is G; if present, X170 is G; if present, X171 is Xaa; if present, X172 is Xaa; if present, X173 is Xaa; X174 is M, A, T C, I, L, S, V, W, Y, F, Q, G, M, R, or P; X175 is T, G, or C; X176 is I, L, M, V, W, A, G, T, P, R, W, S, L, V, F, E, or Y; X177 is I, L, M, W, G, P, S, T, V, A, Y, or F; X178 is F, G, D, W, or L; X179 is A, G, Q, R, or L; X180 is G, H, Q, V, or E; X181 is R, Q, T, P, D, A, S, or H; X182 is G, V, L, W, or Y; X183 is I, Y, G, M, A, S, L, R, V, T, F, W, Q, or P; X184 is Xaa; X185 is Xaa; X186 is V, F, or C; if present, X187 is Y, W, S, T, G, L, F, I, V, M, P, or A; if present, X188 is Xaa; if present, X189 is Xaa; if present, X190 is W, A, G, P, N, S, I, K, T, M, L, N, R, Y, E, V, F; if present, X191 is G; and if present, X192 is G, and (iii) Xaa is any amino acid (SEQ ID NO: 489). In one embodiment, X174 is I, L, M, V, F, Y, W, A, G, P, S, or T; X175 is C, X176 is I, L, M, V, F, Y, W, A, G, P, S, or T; X177 is Y or F; X178 is F; X179 is G; X180 is G; X181 is T; X182 is V; X183 is R; X184 is Xaa; X185 is Xaa; X186 is C; and X187 is L (beta consensus 1; SEQ ID NO: 467). In another embodiment, X177 is A, X182 is I, L, M, V, F, Y, W, A, G, P, S, or T; X183 is I, L, M, V, F, Y, W, A, G, P, S, or T, and X187 is I, L, M, V, F, Y, W, A, G, P, S, or T (beta consensus 2; SEQ ID NO: 490). In another embodiment, X171 is Xaa; X172 is Xaa; X173 is Xaa; X174 is L; X175 is C; X176 is W; X177 is Y; X178 is F; X179 is G; X180 is G; X181 is T; X182 is V; X183 is R; X184 is G; X185 is P; X186 is C; X187 is L; X188 is Xaa; X189 is Xaa; and X190 is I, L, M, V, F, Y, W, A, G, P, S, or T (beta consensus 3; SEQ ID NO: 468). In another embodiment, the IL-18R beta binding moiety has (i) the amino acid sequence of, for example, GGLCWYFGGTVRGPCLGG (beta synthetic construct; SEQ ID NO: 217) or LCWYFGGTVRGPCL (beta synthetic construct; SEQ ID NO: 218), or (ii) an amino acid sequence having 1, 2, 3, or 4 substitution(s), addition(s), and/or deletion(s) relative to an amino acid sequence of any one of (i). In another embodiment, the IL-18R beta binding moiety has (i) the amino acid sequence of, for example, GGEGFFCEYWGVTGLGPCPGINGG (8C12; SEQ ID NO: 219); GGIYGLCSYFGGTVWGLCLGDSGG (9A03; SEQ ID NO: 220); GGGASQCWYFGGTVRGPCLGPIGG (9F07; SEQ ID NO: 221); GGKGHGCWYLGGTGQGPCLGGKGG (11D11; SEQ ID NO: 222); GGGDYLCWYFGGTVRGSCAGTTGG (8H11; SEQ ID NO: 223); GGTSRLCWYFAGTVPGPCLEAIGG (9D06; SEQ ID NO: 224); GGNNFMCWYFGGTVRAPCLQYMGG (9B11; SEQ ID NO: 225); GGGSGICWYFAGTVPGPCLSQTGG (11H07; SEQ ID NO: 226); GGGEKLCWYFGETVRGPCLKWMGG (10E05; SEQ ID NO: 227); GGSSRLCWYFAGTVQGPCLSSLGG (10F10; SEQ ID NO: 228); GGGEKLCWFFGGTVRAPCLNHMGG (10D02; SEQ ID NO: 229); GGKDRRCWYFGGTVQGPCLSSNGG (10E06; SEQ ID NO: 230); GGAAAPCWYFGGTVRGPCLGARGG (8H02; SEQ ID NO: 231); GGFSPLCYYFGGTVRGPCLGGTGG (9A11; SEQ ID NO: 232); GGKGHLCSYFAGTVLGPCLWDTGG (8E01; SEQ ID NO: 233); GGPVRLCWYFQETVRAPCLKFMGG (10006; SEQ ID NO: 234); GGGQYLCWYFGGAVSGTCLDDYGG (8E08; SEQ ID NO: 235); GGSGVLCWYFGGRVPGPCLGQNGG (9A08; SEQ ID NO: 236); GGREKLCVYFGGTVWGPCLDGTGG (9D04; SEQ ID NO: 237); GGGKELCWYFAGTVRGPCLGSIGG (9E01; SEQ ID NO: 238); GGWWGLCSYFGGTVSGPCLTERGG (8G02; SEQ ID NO: 239); GGDSRLCWYFAGTVLGPCLHQIGG (9C05; SEQ ID NO: 240); GGLTGLCSYFGGSVQGPCLAQSGG (9C02; SEQ ID NO: 241); GGYATQCWYFGGHVRGPCLEREGG (8C01; SEQ ID NO: 242); GGKDNRCWYFAGTVLGPCLFGTGG (9G06; SEQ ID NO: 243); GGHGELCWYFGGTVPGPCLDKVGG (9B04; SEQ ID NO: 244); GGDEEQCWYFGGRVRGPCLLENGG (8H07; SEQ ID NO: 245); GGVNNLCWYFGGTVLGPCLEGFGG (8B01; SEQ ID NO: 246); or GGDKRMCVYFGGTVLGPCLQDIGG (8C07; SEQ ID NO: 247); or (ii) an amino acid sequence having 1, 2, 3, or 4 substitution(s), addition(s), and/or deletion(s) relative to an amino acid sequence of any one of (i). In another embodiment, X169, X170, X191, and X192 are absent and examples of the IL-18R beta binding moiety include, but are not limited to, (i) the amino acid sequence of, for example, EGFFCEYWGVTGLGPCPGIN (8C12-G; SEQ ID NO: 248); IYGLCSYFGGTVWGLCLGDS (9A03-G; SEQ ID NO: 249); GASQCWYFGGTVRGPCLGPI (9F07-G; SEQ ID NO: 250); KGHGCWYLGGTGQGPCLGGK (11D11-G; SEQ ID NO: 251); GDYLCWYFGGTVRGSCAGTT (8H11-G; SEQ ID NO: 252); TSRLCWYFAGTVPGPCLEAI (9D06-G; SEQ ID NO: 253); NNFMCWYFGGTVRAPCLQYM (9B11-G; SEQ ID NO: 254); GSGICWYFAGTVPGPCLSQT (11H07-G; SEQ ID NO: 255); GEKLCWYFGETVRGPCLKWM (10E05-G; SEQ ID NO: 256); SSRLCWYFAGTVQGPCLSSL (10F10-G; SEQ ID NO: 257); GEKLCWFFGGTVRAPCLNHM (10D02-G; SEQ ID NO: 258); KDRRCWYFGGTVQGPCLSSN (10E06-G; SEQ ID NO: 259); AAAPCWYFGGTVRGPCLGAR (8H02-G; SEQ ID NO: 260); FSPLCYYFGGTVRGPCLGGT (9A11-G; SEQ ID NO: 261); KGHLCSYFAGTVLGPCLWDT (8E01-G; SEQ ID NO: 262); PVRLCWYFQETVRAPCLKFM (10006-G; SEQ ID NO: 263); GQYLCWYFGGAVSGTCLDDY (8E08-G; SEQ ID NO: 264); SGVLCWYFGGRVPGPCLGQN (9A08-G; SEQ ID NO: 265); REKLCVYFGGTVWGPCLDGT (9D04; SEQ ID NO: 266); GKELCWYFAGTVRGPCLGSI (9E01-G; SEQ ID NO: 267); WWGLCSYFGGTVSGPCLTER (8G02-G; SEQ ID NO: 268); DSRLCWYFAGTVLGPCLHQI (9C05-G; SEQ ID NO: 269); LTGLCSYFGGSVQGPCLAQS (9C02-G; SEQ ID NO: 270); YATQCWYFGGHVRGPCLERE (8C01-G; SEQ ID NO: 271); KDNRCWYFAGTVLGPCLFGT (9G06-G; SEQ ID NO: 272); HGELCWYFGGTVPGPCLDKV (9B04-G; SEQ ID NO: 273); DEEQCWYFGGRVRGPCLLEN (8H07-G; SEQ ID NO: 274); VNNLCWYFGGTVLGPCLEGF (8B01-G; SEQ ID NO: 275); or DKRMCVYFGGTVLGPCLQDI (8C07-G; SEQ ID NO: 276); or (ii) an amino acid sequence having 1, 2, 3, or 4 substitution(s), addition(s), and/or deletion(s) relative to an amino acid sequence of any one of (i). In another embodiment, X169, X170, X171, X190, X191, and X192 are absent and examples of the IL-18R beta binding moiety include, but are not limited to, (i) the amino acid sequence of GGICWYFGGQVSSKCLGG (3G06; SEQ ID NO: 277); GGLCSYFAGTLLGPCLGG (3G05; SEQ ID NO: 278); GGLCSFFQGTVRATCLGG (3E04; SEQ ID NO: 279); GGSCLYFGGTVSGRCLGG (3D12; SEQ ID NO: 280); GGSCVYFGGRVVGPCFGG (3H04; SEQ ID NO: 281); GGVCWYFGHTVRSYCIGG (3H11; SEQ ID NO: 282); GGWCRAFAGTVTYPCVGG (3E06; SEQ ID NO: 283); GGYCFAGRGPWRAECMGG (3B06; SEQ ID NO: 284); or GGYCPADLQDYFMDCVGG (1D11; SEQ ID NO: 285), or (ii) an amino acid sequence having 1, 2, 3, or 4 substitution(s), addition(s), and/or deletion(s) relative to an amino acid sequence of any one of (i). In another embodiment, X169, X170, X171, X172, X173, X188, and X189 X190, X191, and X192 are absent and examples of the IL-18R beta binding moiety include, but are not limited to, (i) the amino acid sequence of ICWYFGGQVSSKCL (3G06-G; SEQ ID NO: 286); LCSYFAGTLLGPCL (3G05-G; SEQ ID NO: 287); LCSFFQGTVRATCL (3E04-G; SEQ ID NO: 288); SCLYFGGTVSGRCL (3D12-G; SEQ ID NO: 289); SCVYFGGRVVGPCF (3H04-G; SEQ ID NO: 290); VCWYFGHTVRSYCI (3H11-G; SEQ ID NO: 291); WCRAFAGTVTYPCV (3E06-G; SEQ ID NO: 292); YCFAGRGPWRAECM (3B06-G; SEQ ID NO: 293); or YCPADLQDYFMDCV (1D11-G; SEQ ID NO: 294), or (ii) an amino acid sequence having 1, 2, 3, or 4 substitution(s), addition(s), and/or deletion(s) relative to an amino acid sequence of any one of (i). In another embodiment, X169, X170, X189, X190, X191, and X192 are absent and examples of the IL-18R beta binding moiety include, but are not limited to, (i) the amino acid sequence of GGGCTPVFAGRGMWCVGG (3A06; SEQ ID NO: 295) or GGRCGRAFGGQVAFCFGG (3A05; SEQ ID NO: 296), or (ii) an amino acid sequence having 1, 2, 3, or 4 substitution(s), addition(s), and/or deletion(s) relative to an amino acid sequence of any one of (i). In another embodiment, X169, X170, X171, X172, X187, X188, X189, X190, X191, and X192 are absent and examples of the IL-18R beta binding moiety include, but are not limited to, (i) the amino acid sequence of GCTPVFAGRGMWCV (3A06-G; SEQ ID NO: 297) or RCGRAFGGQVAFCF (3A05-G; SEQ ID NO: 298), or (ii) an amino acid sequence having 1, 2, 3, or 4 substitution(s), addition(s), and/or deletion(s) relative to an amino acid sequence of any one of (i).

### IL-18R Beta Sequence Family 3

In another aspect, an IL-18R beta binding moiety can have the amino acid sequence of X193-X194-X195-X196-X197-X198-X199-X200-X201-X202-X203-X204-X205-X206-X207-X208-X209-X210-X211-X212-X213-X214-X215-X216; wherein: (i) one or more of X193, X194, X195, X196, X197, X198, X212, X213, X214, X215, and X216 are optional, (ii) if present, X193 is G; if present, X194 is G; if present, X195 is G, E, F, H, S, T, L, M, A, Q, L, R, S, M, N, X, W, L, or P; if present, X196 is R, G, N, M, L, T, W, S, I, Q, K, or Y; if present, X197 is M, G, V, D, R, A, I, S, K, N, L, or W; if present, X198 is Xaa; X199 is K, M, P, or G; X200 is Y, C, N, L, P, R, D, F, E, K, or A; X201 is A, P, W, F, or L; X202 is F, L, M, I, V, Y, W, A, G, P, S, or T; X203 is P, Q, S, G, F, D, R, T, or A; X204 is D; X205 is H; X206 is P, W, L, V, F, A, D, R, Q, or M; X207 is R, S, G, Y, or D; X208 is L, E, D, or G; X209 is W, A, G, P, S, T, C, V, I, L, M, F, Y, H, or Q; X210 is M, F, Y, A, G, P, S, T, L, W, V, or I; X211 is F or C; if present, X212 is Xaa; if present, X213 is G, R, F, H, S, D, V, A, Q, P, or K; if present, X214 is G, Q, R, P, F, S, H, T, M, N, or L; if present, X215 is G; and if present, X216 is G, and (iii) Xaa is any amino acid (SEQ ID NO: 491). In one embodiment, X198is Xaa; X199 is P; X200is D, E, or N; X201 is W; X202 is I, L, M, V, F, Y, W, A, G, P, S, or T; X203 is S; X204 is D; X205 is H; X206 is F; X207 is G or S; X208 is D or E; X209 is I, L, M, V, F, Y, W, A, G, P, S, or T; X210 is I, L, M, V, F, Y, W, A, G, P, S, or T; and X212 is Xaa (beta family 3 consensus; SEQ ID NO: 470). In another embodiment, the IL-18R beta binding moiety has (i) the amino acid sequence of, for example, GGWKACPDWLSDHMSEIWCHQGG (13A03; SEQ ID NO: 299); GGENVCPEWLSDHFSEMLCTRQGG (13H04; SEQ ID NO: 300); GGFMDCPEWLSDHFYEMICHFRGG (13C08; SEQ ID NO: 301); GGHLRCPKWLADHDSEMLCFRPGG (13A01; SEQ ID NO: 302); GGSTGCPEWLSDHFYEMLCNFQGG (13H10; SEQ ID NO: 303); GGTLGCPNWLSDHWGEILCSGFGG (13G02; SEQ ID NO: 304); GGLWACPDWLADHRYEMLCYHSGG (13B02; SEQ ID NO: 305); GGMNICPYWLSDHFSEMLCTSQGG (13G05; SEQ ID NO: 306); GGASSCPDWLSDHFGEILCMSHGG (13D07; SEQ ID NO: 307); GGQISCPNWLSDHFGEILCYRRGG (13A05; SEQ ID NO: 308); GGLQVCPNWLSDHLGEFWCLGRGG (13H03; SEQ ID NO: 309); GGQSRCPKWLSDHDGEMLCTRTGG (13C09; SEQ ID NO: 310); GGRNMCPRWLSDHFGEMLCSDNGG (13E08; SEQ ID NO: 311); GGSNKCPPWLSDHQYEVLCQVMGG (13C10; SEQ ID NO: 312); GGMQNCPNWLSDHQGEMLCMVSGG (13E06; SEQ ID NO: 313); GGNRLCPAWLSDHFGEQLCMALGG (13H02; SEQ ID NO: 314); GGXKRCPKWLSDHRGEVVCXQNGG (13E03; SEQ ID NO: 315); GGLNLCPEWLSDHWGEFLCFPRGG (13G08; SEQ ID NO: 316); GGGNWCPKWLSDHFDEVICTGFGG (13F08; SEQ ID NO: 317); GGRNMCPRWLSDHFGEILCFDNGG (13B12; SEQ ID NO: 318); or GGPYGCPNWLSDHFGELLCKKTGG (13H07; SEQ ID NO: 319); or (ii) an amino acid sequence having 1, 2, 3, or 4 substitution(s), addition(s), and/or deletion(s) relative to an amino acid sequence of any one of (i). In another embodiment, X193, X194, X215, and X216 are absent and examples of the IL-18R beta binding moiety include, but are not limited to, (i) the amino acid sequence of WKACPDWLSDHMSEIWCHQ (13A03-G; SEQ ID NO: 320); ENVCPEWLSDHFSEMLCTRQ (13H04-G; SEQ ID NO: 321); FMDCPEWLSDHFYEMICHFR (13C08-G; SEQ ID NO: 322); HLRCPKWLADHDSEMLCFRP (13A01-G; SEQ ID NO: 323); STGCPEWLSDHFYEMLCNFQ (13H10-G; SEQ ID NO: 324); TLGCPNWLSDHWGEILCSGF (13G02-G; SEQ ID NO: 325); LWACPDWLADHRYEMLCYHS (13B02-G; SEQ ID NO: 326); MNICPYWLSDHFSEMLCTSQ (13G05-G; SEQ ID NO: 327); ASSCPDWLSDHFGEILCMSH (13D07-G; SEQ ID NO: 328); QISCPNWLSDHFGEILCYRR (13A05-G; SEQ ID NO: 329); LQVCPNWLSDHLGEFWCLGR (13H03-G; SEQ ID NO: 330); QSRCPKWLSDHDGEMLCTRT (13C09-G; SEQ ID NO: 331); RNMCPRWLSDHFGEMLCSDN (13E08-G; SEQ ID NO: 332); SNKCPPWLSDHQYEVLCQVM (13C10-G; SEQ ID NO: 333); MQNCPNWLSDHQGEMLCMVS (13E06-G; SEQ ID NO: 334); NRLCPAWLSDHFGEQLCMAL (13H02-G; SEQ ID NO: 335); XKRCPKWLSDHRGEVVCXQN (13E03-G; SEQ ID NO: 336); LNLCPEWLSDHWGEFLCFPR (13G08-G; SEQ ID NO: 337); GNWCPKWLSDHFDEVICTGF (13F08-G; SEQ ID NO: 338); RNMCPRWLSDHFGEILCFDN (13B12-G; SEQ ID NO: 339); or PYGCPNWLSDHFGELLCKKT (13H07-G; SEQ ID NO: 340); or (ii) an amino acid sequence having 1, 2, 3, or 4 substitution(s), addition(s), and/or deletion(s) relative to an amino acid sequence of any one of (i). In another embodiment, X193, X194, X195, X214, X215, and X216 are absent and examples of the IL-18R beta binding moiety include, but are not limited to, (i) the amino acid sequence of GGCPNWLSDHLSEIWCGG (5C09; SEQ ID NO: 341); GGCPLWLGDHVGDLVCGG (5C05; SEQ ID NO: 342); GGCPLWMSDHFYDMVCGG (5G03; SEQ ID NO: 343); GGCPPWFFDHASEFICGG (7C03; SEQ ID NO: 344); GGCPRWLDDHDGGYICGG (5G05; SEQ ID NO: 345); GGCPDFLRDHRSEIICGG (5G07; SEQ ID NO: 346); or GGCGFLLTDHPSLHVCGG (5F12; SEQ ID NO: 347); or (ii) an amino acid sequence having 1, 2, 3, or 4 substitution(s), addition(s), and/or deletion(s) relative to an amino acid sequence of any one of (i). In another embodiment, X193, X194, X195, X196, X197, X212, X213, X214, X215, and X216 are absent and examples of the IL-18R beta binding moiety include, but are not limited to, (i) the amino acid sequence of CPNWLSDHLSEIWC (5C09-G; SEQ ID NO: 348); CPLWLGDHVGDLVC (5C05-G; SEQ ID NO: 349); CPLWMSDHFYDMVC (5G03-G; SEQ ID NO: 350); CPPWFFDHASEFIC (7C03-G; SEQ ID NO: 351); CPRWLDDHDGGYIC (5G05-G; SEQ ID NO: 352); CPDFLRDHRSEIIC (5G07-G; SEQ ID NO: 353); or CGFLLTDHPSLHVC (5F12-G; SEQ ID NO: 354); or (ii) an amino acid sequence having 1, 2, 3, or 4 substitution(s), addition(s), and/or deletion(s) relative to an amino acid sequence of any one of (i). In another embodiment, X193, X214, X215, and X216 are absent and examples of the IL-18R beta binding moiety include, but are not limited to, (i) the amino acid sequence of GGRMCKYAFPDHPRLCLFGG (7C10; SEQ ID NO: 355) or (ii) an amino acid sequence having 1, 2, 3, or 4 substitution(s), addition(s), and/or deletion(s) relative to the amino acid sequence of (i). In another embodiment, X193, X194, X195, X212, X213, X214, X215, and X216 are absent and examples of the IL-18R beta binding moiety include, but are not limited to, (i) the amino acid sequence of RMCKYAFPDHPRLCLF (7C10-G; SEQ ID NO: 356) or (ii) an amino acid sequence having 1, 2, 3, or 4 substitution(s), addition(s), and/or deletion(s) relative to the amino acid sequence of (i). In another embodiment, X193, X194, X195, X196, X215, and X216 are absent and examples of the IL-18R beta binding moiety include, but are not limited to, (i) the amino acid sequence of GGMCPFQDHWREVWCWGG (7E01; SEQ ID NO: 357) or (ii) an amino acid sequence having 1, 2, 3, or 4 substitution(s), addition(s), and/or deletion(s) relative to the amino acid sequence of (i). In another embodiment, X193, X194, X195, X196, X197, X197, X213, X214, X215, and X216 are absent and examples of the IL-18R beta binding moiety include, but are not limited to, (i) the amino acid sequence of MCPFQDHWREVWCW (7E01-G; SEQ ID NO: 358) or (ii) an amino acid sequence having 1, 2, 3, or 4 substitution(s), addition(s), and/or deletion(s) relative to the amino acid sequence of (i).

In another embodiment, the IL-18R beta binding moiety has (i) the amino acid sequence of, for example, GGENVCPEWLSDHFSEMLCTRQGG (5C09_AM1; SEQ ID NO: 300); GGFMDCPEWLSDHFYEMICHFRGG (5C09_AM2; SEQ ID NO: 301); GGHLRCPKWLADHDSEMLCFRPGG (5C09_AM3; SEQ ID NO: 302); GGSTGCPEWLSDHFYEMLCNFQGG (5C09_AM4; SEQ ID NO: 303); GGTLGCPNWLSDHWGEILCSGFGG (5C09 AMS; SEQ ID NO: 304); or (ii) an amino acid sequence having 1, 2, 3, or 4 substitution(s), addition(s), and/or deletion(s) relative to an amino acid sequence of any one of (i). In another embodiment, the IL-18R beta binding moiety has (i) the amino acid sequence of, for example, ENVCPEWLSDHFSEMLCTRQ (5C09_AM1-G; SEQ ID NO: 321); FMDCPEWLSDHFYEMICHFR (5C09_AM2-G; SEQ ID NO: 322); HLRCPKWLADHDSEMLCFRP (5C09_AM3-G; SEQ ID NO: 323); STGCPEWLSDHFYEMLCNFQ (5C09_AM4-G; SEQ ID NO: 324); TLGCPNWLSDHWGEILCSGF (5C09_AM5-G; SEQ ID NO: 325); or (ii) an amino acid sequence having 1, 2, 3, or 4 substitution(s), addition(s), and/or deletion(s) relative to an amino acid sequence of any one of (i).

### IL-18R Beta Sequence Family 4

In another aspect, an IL-18R beta binding moiety can have the amino acid sequence of the amino acid sequence of X217-X218-X219-X220-X221-X222-X223-X224-X225-X226-X227-X228-X229-X230-X231-X232-X233-X234-X235-X236, wherein: (i) one or more of X217, X218, X219, X233, X234, X235, and X236 are optional; (ii) if present, X217 is G, if present, X218 is G; if present, X219 is F, H, W, Y, or G; X220 is Xaa; X221 is L or C, X222 is I, V, F, Y, W, A, G, P, S, T, L, M, or C; X223 is N or Q; X224 is D; X225 is H; X226 is P or G; X227 is E or W; X228 is V, F, Y, W, A, G, P, S, T, L, I, or M; X229 is M, V, F, Y, W, A, G, P, S, T, H, L, or I; X230 is C or R; X231 is I, M, F, Y, W, A, G, P, S, T, V, L, or E; X232 is Xaa; if present, X233 is Xaa; if present, X234 is Xaa; if present, X235 is G, and if present, X236 is G; and (iii) Xaa is any amino acid (SEQ ID NO: 492). In one embodiment, X219 is F, H, W, or Y; X220 is Xaa, X221 is C, X222 is I, L, M, V, F, Y, W, A, G, P, S, or T; X223 is N; X224 is D; X225 is H; X226 is P; X227 is E; X228 is I, L, M, V, F, Y, W, A, G, P, S, or T; X229 is I, L, M, V, F, Y, W, A, G, P, S, or T; X230 is C; X231 is I, L, M, V, F, Y, W, A, G, P, S, or T; X232 is Xaa; X233 is Xaa; and X234 is Xaa, wherein Xaa is any amino acid (beta F4 consensus; SEQ ID NO: 471). In another embodiment, X235 and X236 are absent and examples of the IL-18R beta binding moiety include, but are not limited to, (i) the amino acid sequence of GGWHCLNDHPELHCVRGG (6B03; SEQ ID NO: 359) or GGYTCMNDHPEILCLTGG (6E05; SEQ ID NO: 207); or (ii) an amino acid sequence having 1, 2, 3, or 4 substitution(s), addition(s), and/or deletion(s) relative to an amino acid sequence of any one of (i). In another embodiment, X217, X218, X233, X234, X235, and X236 are absent and examples of the IL-18R beta binding moiety include, but are not limited to, (i) the amino acid sequence of WHCLNDHPELHCVR (6B03-G; SEQ ID NO: 360) or YTCMNDHPEILCLT (6E05-G; SEQ ID NO: 212); or (ii) an amino acid sequence having 1, 2, 3, or 4 substitution(s), addition(s), and/or deletion(s) relative to an amino acid sequence of any one of (i). In another embodiment, X217 is absent and examples of the IL-18R beta binding moiety include, but are not limited to, (i) the amino acid sequence of GGILCQDHGWMIRECLLGG (7A04; SEQ ID NO: 361) or (ii) an amino acid sequence having 1, 2, 3, or 4 substitution(s), addition(s), and/or deletion(s) relative to the amino acid sequence of (i). In another embodiment, X217, X218, X219, X235, and X236 are absent and examples of the IL-18R beta binding moiety include, but are not limited to, (i) the amino acid sequence of ILCQDHGWMIRECLL (7A04-G; SEQ ID NO: 362) or (ii) an amino acid sequence having 1, 2, 3, or 4 substitution(s), addition(s), and/or deletion(s) relative to the amino acid sequence of (i).

### IL-18R beta Sequence Family 5

In another aspect, an IL-18R beta binding moiety can have, in some instances, (i) the amino acid sequence of, for example, GGYALFGDGNWPWWGG (6C08; SEQ ID NO: 363); GGLQWCFGGMVPCTLNGG (4A07; SEQ ID NO: 364); GGFVACLQDHITACRWFGG (3B04; SEQ ID NO: 365); GGNCFSAKSDWLFWMCEGG (4E07; SEQ ID NO: 366); GGSCSWGYDWLHNEWCPGG (4G08; SEQ ID NO: 367); GGYCQSVWMQQHFMSCHGG (4H10; SEQ ID NO: 368); GGCPVWLLDHQDEFLCGG (1G12; SEQ ID NO: 369); GGGSPCDDHPGWSPCYLVGG (2C07; SEQ ID NO: 370); GGYSCYLMEPGDPYICYSGG (2B07; SEQ ID NO: 371); GGQPCTAWRPGGKGFCRSGG (4H06; SEQ ID NO: 372); GGVGCPSWVPGALGFCREGG (4G05; SEQ ID NO: 373); GGPYCLWGSGTYFDCMRGG (7G02; SEQ ID NO: 203); GGGLCSWVGTDGVCYVGG (6G02; SEQ ID NO: 374); GGNRMCSRYPGYYFCVQSGG (7F12; SEQ ID NO: 375); GGWPCDYVDSGGTCIIGG (6D03; SEQ ID NO: 376); GQCYRDYVIHDFVCQGG (5G04; SEQ ID NO: 377); GGCVFPENSWVWDFYCGG (5E05; SEQ ID NO: 378); GGTCSFLQQKIVDLCTGG (5E07; SEQ ID NO: 379); GGSPCRFRSWYDDRCLVGG (7E06; SEQ ID NO: 380); or GGSCDFLQERILEICWGG (5G11; SEQ ID NO: 381); or (ii) an amino acid sequence having 1, 2, 3, or 4 substitution(s), addition(s), and/or deletion(s) relative to an amino acid sequence of any one of (i).

An IL-18R beta binding moiety can have, in other instances, (i) an amino acid sequence of, for example, YALFGDGNWPWW (6C08-G; SEQ ID NO: 382); LQWCFGGMVPCTLN (4A07-G; SEQ ID NO: 383); FVACLQDHITACRWF (3B04-G; SEQ ID NO: 384); NCFSAKSDWLFWMCE (4E07-G; SEQ ID NO: 385); SCSWGYDWLHNEWCP (4G08-G; SEQ ID NO: 386); YCQSVWMQQHFMSCH (4H10-G; SEQ ID NO: 387); CPVWLLDHQDEFLC (1G12-G; SEQ ID NO: 388); GSPCDDHPGWSPCYLV (2C07-G; SEQ ID NO: 389); YSCYLMEPGDPYICYS (2B07-G; SEQ ID NO: 390); QPCTAWRPGGKGFCRS (4H06-G; SEQ ID NO: 391); VGCPSWVPGALGFCRE (4G05-G; SEQ ID NO: 392); PYCLWGSGTYFDCMR (7G02-G; SEQ ID NO: 205); GLCSWVGTDGVCYV (6G02-G; SEQ ID NO: 393); NRMCSRYPGYYFCVQS (7F12-G; SEQ ID NO: 394); WPCDYVDSGGTCII (6D03-G; SEQ ID NO: 395); QCYRDYVIHDFVCQ (5G04-G; SEQ ID NO: 396); CVFPENSWVWDFYC (5E05-G; SEQ ID NO: 397); TCSFLQQKIVDLCT (5E07-G; SEQ ID NO: 398); SPCRFRSWYDDRCLV (7E06-G; SEQ ID NO: 399); or SCDFLQERILEICW (5G11-G; SEQ ID NO: 400); or (ii) an amino acid sequence having 1, 2, 3, or 4 substitution(s), addition(s), and/or deletion(s) relative to an amino acid sequence of any one of (i).

### IL-18R Binding Moieties Comprising an IL-18R Alpha Binding Moiety and an IL-18R Beta Binding Moiety

In another aspect, provided herein is an IL-18R binding moiety comprising an IL-18R alpha binding moiety and an IL-18R beta binding moiety. An IL-18R binding moiety can, in some instances, be an IL-18R agonist. An IL-18R binding moiety can, in other instances, be an IL-18R antagonist. Alternatively, or in addition, the binding moiety can further comprise a linker. In one non-limiting example, the C terminus of the IL-18R alpha binding moiety can be coupled to the N-terminus of the IL-18R beta binding moiety by, for example, a linker (e.g., a C-N dimer). In one non-limiting example, the N terminus of the IL-18R alpha binding moiety can be coupled to the C terminus of the IL-18R beta binding moiety by, for example, a linker (e.g., a N-C dimer). In another non-limiting example, the C terminus of the IL-18R alpha binding moiety can be coupled to the C terminus of the IL-18R beta binding moiety by, for example, a linker (e.g., a C-C dimer). In yet another non-limiting example, the N terminus of the IL-18R alpha binding moiety can be coupled to the N terminus of the IL-18R beta binding moiety by, for example, a linker (e.g., a N-N dimer). A linker that can be used in such polypeptides includes, but is not limited to, a (Proline-Alanine)8 linker (“(PA)8”; SEQ ID NO: 401), or a glycine senile linker (e.g., (GS)n, wherein n=0, 1, 2, 3, 4, 5, 6, 7, 8, 9, or 10 (SEQ ID NO: 402). Optionally, a linker can further comprise a triazole. Non-limiting examples of linkers that can be used in such polypeptides further include, but are not limited to, a linker of Formula I, II, III, IV, V, or VI. In one example, the linker is a compound of formula I. In another example, the linker is a compound of formula II. In another example, the linker is a compound of formula III. In another example, the linker is a compound of formula IV. In another example, the linker is a compound of formula V. In another example, the linker is a compound of formula VI. Each of the IL-18R binding moieties comprising an IL-18R alpha binding moiety and an IL-18R beta binding moiety were found to bind to IL-18R alpha with a K_{D} of less than 10 μM (data not shown).

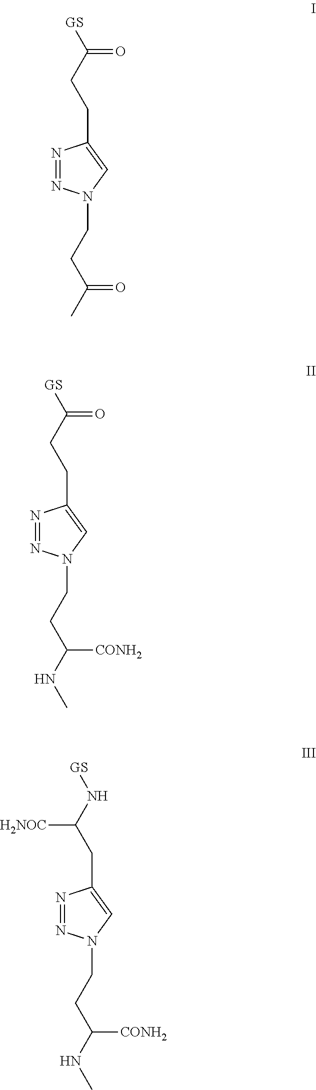

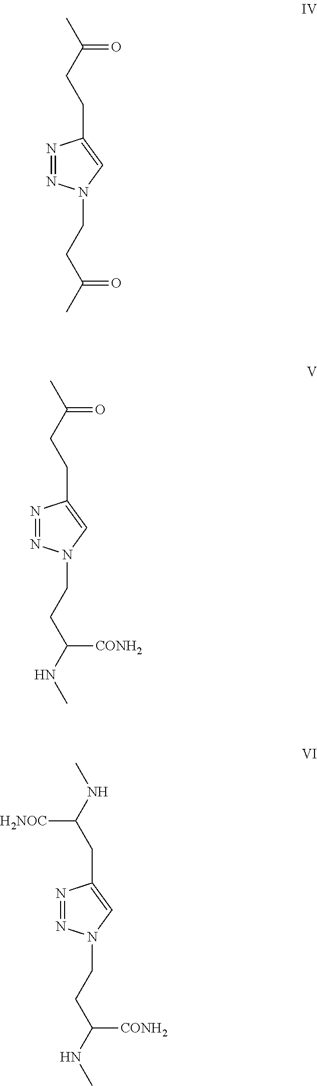 [SMILES: CC(=O)CCc1cn(CCC(C)=O)nn1 ; CNC(CCn1cc(CCC(C)=O)nn1)C(N)=O ; CNC(CCn1cc(CC(NC)C(N)=O)nn1)C(N)=O]

In one instance, an IL-18R binding moiety (dimer 1) comprises an IL-18R alpha binding moiety having the amino acid sequence of GGICWVPFHYLDELMCTGG (SEQ ID NO: 2) or an amino acid sequence having 1, 2, 3, or 4 substitution(s), addition(s), and/or deletion(s) relative to GGICWVPFHYLDELMCTGG (SEQ ID NO: 2), and an IL-18R beta binding moiety having the amino acid sequence of GGCPNWLSDHLSEIWCGG (SEQ ID NO: 341) or an amino acid sequence having 1, 2, 3, or 4 substitution(s), addition(s), and/or deletion(s) relative to GGCPNWLSDHLSEIWCGG (SEQ ID NO: 341). In another instance, an IL-18R binding moiety (dimer 1) comprises an IL-18R alpha binding moiety having the amino acid sequence of ICWVPFHYLDELMCT (SEQ ID NO: 17) or an amino acid sequence having 1, 2, 3, or 4 substitution(s), addition(s), and/or deletion(s) relative to ICWVPFHYLDELMCT (SEQ ID NO: 17), and an IL-18R beta binding moiety having the amino acid sequence of CPNWLSDHLSEIWC (SEQ ID NO: 348) or an amino acid sequence having 1, 2, 3, or 4 substitution(s), addition(s), and/or deletion(s) relative to CPNWLSDHLSEIWC (SEQ ID NO: 348). Such dimers can, optionally, further include a linker and be in any orientation such as N-N, C-C, C-N, or N-C. In one non-limiting example, dimer 1 is in an N-C orientation and includes a linker of formula II with the Gly-Ser dipeptide removed (i.e., a linker of formula V).

In another instance, an IL-18R binding moiety (dimer 2) comprises an IL-18R alpha binding moiety having the amino acid sequence of GGFFECFYSPGHEWCIPSGG (SEQ ID NO: 126) or an amino acid sequence having 1, 2, 3, or 4 substitution(s), addition(s), and/or deletion(s) relative to GGFFECFYSPGHEWCIPSGG (SEQ ID NO: 126), and an IL-18R beta binding moiety having the amino acid sequence of GGCPNWLSDHLSEIWCGG (SEQ ID NO: 341) or an amino acid sequence having 1, 2, 3, or 4 substitution(s), addition(s), and/or deletion(s) relative to GGCPNWLSDHLSEIWCGG (SEQ ID NO: 341). In another instance, an IL-18R binding moiety (dimer 2) comprises an IL-18R alpha binding moiety having the amino acid sequence of FFECFYSPGHEWCIPS (SEQ ID NO: 127) or an amino acid sequence having 1, 2, 3, or 4 substitution(s), addition(s), and/or deletion(s) relative to FFECFYSPGHEWCIPS (SEQ ID NO: 127), and an IL-18R beta binding moiety having the amino acid sequence of CPNWLSDHLSEIWC (SEQ ID NO: 348) or an amino acid sequence having 1, 2, 3, or 4 substitution(s), addition(s), and/or deletion(s) relative to CPNWLSDHLSEIWC (SEQ ID NO: 348). Such dimers can, optionally, further include a linker and be in any orientation such as N-N, C-C, C-N, or N-C. In one non-limiting example, dimer 2 is in an N-C orientation and includes a linker of formula II with the Gly-Ser dipeptide removed (i.e., a linker of formula V).

In another instance, an IL-18R binding moiety (dimer 3) comprises an IL-18R alpha binding moiety having the amino acid sequence of GGFFECFYSPGHEWCIPSGG (SEQ ID NO: 126) or an amino acid sequence having 1, 2, 3, or 4 substitution(s), addition(s), and/or deletion(s) relative to GGFFECFYSPGHEWCIPSGG (SEQ ID NO: 126), and an IL-18R beta binding moiety having the amino acid sequence of GGLCWYFGGTVRGPCLGG (SEQ ID NO: 217) or an amino acid sequence having 1, 2, 3, or 4 substitution(s), addition(s), and/or deletion(s) relative to GGLCWYFGGTVRGPCLGG (SEQ ID NO: 217). In another instance, an IL-18R binding moiety (dimer 3) comprises an IL-18R alpha binding moiety having the amino acid sequence of FFECFYSPGHEWCIPS (SEQ ID NO: 127) or an amino acid sequence having 1, 2, 3, or 4 substitution(s), addition(s), and/or deletion(s) relative to FFECFYSPGHEWCIPS (SEQ ID NO: 127), and an IL-18R beta binding moiety having the amino acid sequence of LCWYFGGTVRGPCL (SEQ ID NO: 218) or an amino acid sequence having 1, 2, 3, or 4 substitution(s), addition(s), and/or deletion(s) relative to LCWYFGGTVRGPCL (SEQ ID NO: 218). Such dimers can, optionally, further include a linker and be in any orientation such as N-N, C-C, C-N, or N-C. In one non-limiting example, dimer 3 is in an N-C orientation and includes a linker of formula II with the Gly-Ser dipeptide removed (i.e., a linker of formula V).

In another instance, an IL-18R binding moiety (dimer 4) comprises an IL-18R alpha binding moiety having the amino acid sequence of GGICWVPFHYLDELMCTGG (SEQ ID NO: 2) or an amino acid sequence having 1, 2, 3, or 4 substitution(s), addition(s), and/or deletion(s) relative to GGICWVPFHYLDELMCTGG (SEQ ID NO: 2), and an IL-18R beta binding moiety having the amino acid sequence of GGLCWYFGGTVRGPCLGG (SEQ ID NO: 217) or an amino acid sequence having 1, 2, 3, or 4 substitution(s), addition(s), and/or deletion(s) relative to GGLCWYFGGTVRGPCLGG (SEQ ID NO: 217). In another instance, an IL-18R binding moiety (dimer 4) comprises an IL-18R alpha binding moiety having the amino acid sequence of ICWVPFHYLDELMCT (SEQ ID NO: 17) or an amino acid sequence having 1, 2, 3, or 4 substitution(s), addition(s), and/or deletion(s) relative to ICWVPFHYLDELMCT (SEQ ID NO: 17), and an IL-18R beta binding moiety having the amino acid sequence of LCWYFGGTVRGPCL (SEQ ID NO: 218) or an amino acid sequence having 1, 2, 3, or 4 substitution(s), addition(s), and/or deletion(s) relative to LCWYFGGTVRGPCL (SEQ ID NO: 218). Such dimers can, optionally, further include a linker of, for example, any one of Formulas I-VI, and be in any orientation such as N-N, C-C, C-N, or N-C.

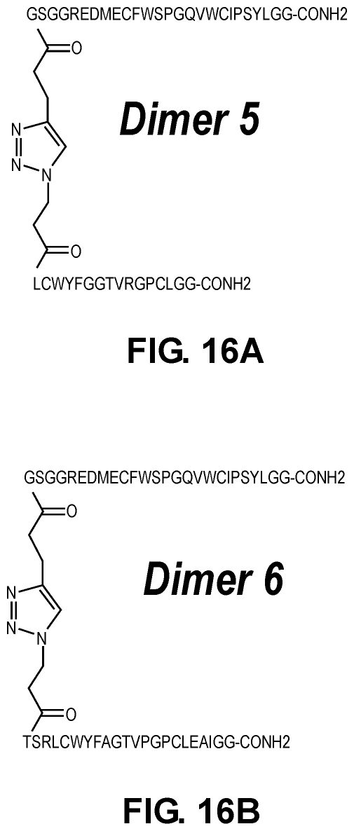

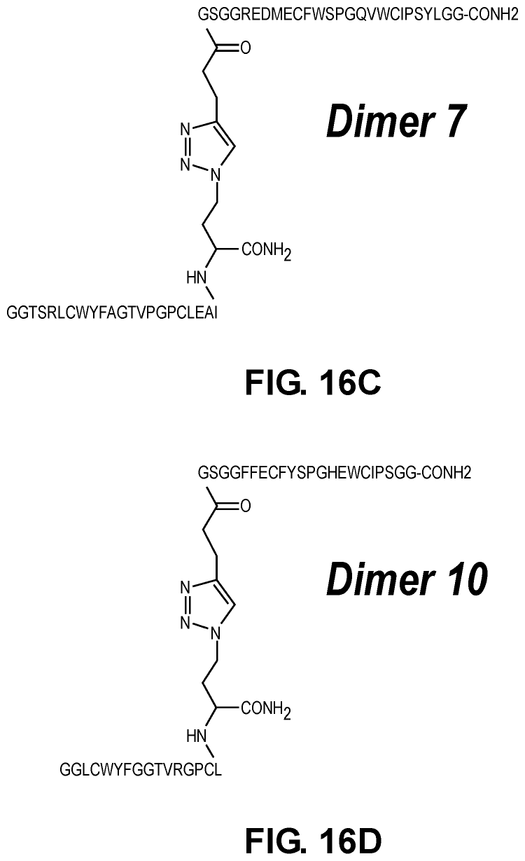

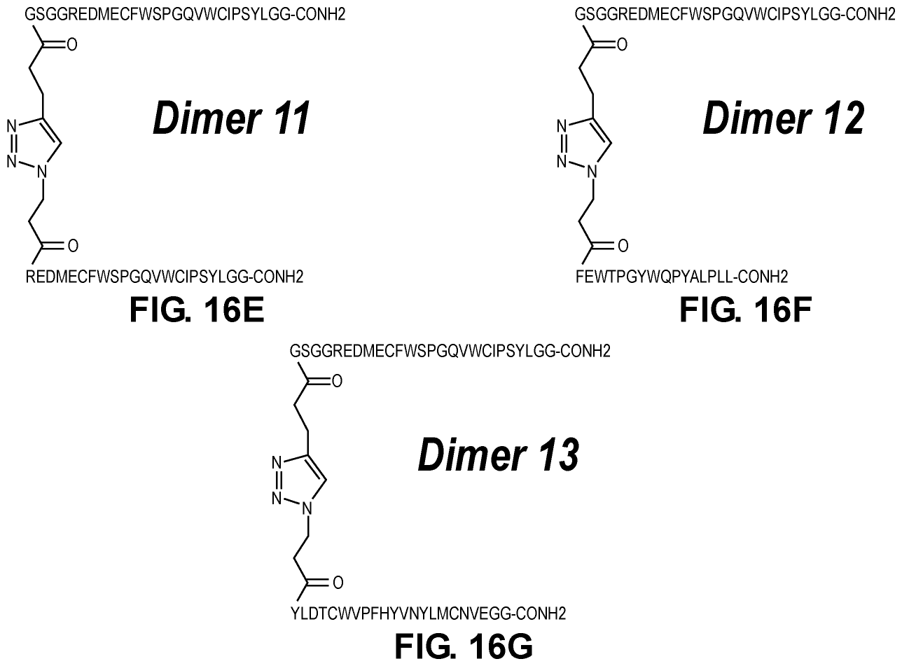

In another instance, an IL-18R binding moiety (dimer 5) comprises an IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and an IL-18R beta binding moiety having the amino acid sequence of GGLCWYFGGTVRGPCLGG-CONH2 (SEQ ID NO: 404) or an amino acid sequence having 1, 2, 3, or 4 substitution(s), addition(s), and/or deletion(s) relative to GGLCWYFGGTVRGPCLGG-CONH2 (SEQ ID NO: 404). In another instance, an IL-18R binding moiety (dimer 5) comprises an IL-18R alpha binding moiety having the amino acid sequence of REDMECFWSPGQVWCIPSYL-CONH2 (SEQ ID NO: 405) or an amino acid sequence having 1, 2, 3, or 4 substitution(s), addition(s), and/or deletion(s) relative to REDMECFWSPGQVWCIPSYL-CONH2 (SEQ ID NO: 405), and an IL-18R beta binding moiety having the amino acid sequence of LCWYFGGTVRGPCL-CONH2 (SEQ ID NO: 406) or an amino acid sequence having 1, 2, 3, or 4 substitution(s), addition(s), and/or deletion(s) relative to GGLCWYFGGTVRGPCLGG-CONH2 (SEQ ID NO: 404). Such dimers can, optionally, further include a linker and be in any orientation such as N-N, C-C, C-N, or N-C. FIG. 16A provides a representative configuration of dimer 5 in an N-N orientation with a linker of formula I.

In another instance, an IL-18R binding moiety (dimer 6) comprises an IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and an IL-18R beta binding moiety having the amino acid sequence of GGTSRLCWYFAGTVPGPCLEAIGG-CONH2 (SEQ ID NO: 407) or an amino acid sequence having 1, 2, 3, or 4 substitution(s), addition(s), and/or deletion(s) relative to GGTSRLCWYFAGTVPGPCLEAIGG-CONH2 (SEQ ID NO: 407). In another instance, an IL-18R binding moiety (dimer 6) comprises an IL-18R alpha binding moiety having the amino acid sequence of REDMECFWSPGQVWCIPSYL-CONH2 (SEQ ID NO: 405) or an amino acid sequence having 1, 2, 3, or 4 substitution(s), addition(s), and/or deletion(s) relative to REDMECFWSPGQVWCIPSYL-CONH2 (SEQ ID NO: 405), and an IL-18R beta binding moiety having the amino acid sequence of TSRLCWYFAGTVPGPCLEAI-CONH2 (SEQ ID NO: 408) or an amino acid sequence having 1, 2, 3, or 4 substitution(s), addition(s), and/or deletion(s) relative to TSRLCWYFAGTVPGPCLEAI-CONH2 (SEQ ID NO: 408). Such dimers can, optionally, further include a linker and be in any orientation such as N-N, C-C, C-N, or N-C. FIG. 16B provides a representative configuration of dimer 6 in an N-N orientation with a linker.

In another instance, an IL-18R binding moiety (dimer 7) comprises an IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and an IL-18R beta binding moiety having the amino acid sequence of GGTSRLCWYFAGTVPGPCLEAI (SEQ ID NO: 409) or an amino acid sequence having 1, 2, 3, or 4 substitution(s), addition(s), and/or deletion(s) relative to GGTSRLCWYFAGTVPGPCLEAI (SEQ ID NO: 409). In another instance, an IL-18R binding moiety (dimer 7) comprises an IL-18R alpha binding moiety having the amino acid sequence of REDMECFWSPGQVWCIPSYL-CONH2 (SEQ ID NO: 405) or an amino acid sequence having 1, 2, 3, or 4 substitution(s), addition(s), and/or deletion(s) relative to REDMECFWSPGQVWCIPSYL-CONH2 (SEQ ID NO: 405), and an IL-18R beta binding moiety having the amino acid sequence of TSRLCWYFAGTVPGPCLEAI (SEQ ID NO: 253) or an amino acid sequence having 1, 2, 3, or 4 substitution(s), addition(s), and/or deletion(s) relative to TSRLCWYFAGTVPGPCLEAI (SEQ ID NO: 253). Such dimers can, optionally, further include a linker and be in any orientation such as N-N, C-C, C-N, or N-C. FIG. 16C provides a representative configuration of dimer 7 in an N-C orientation with a linker.

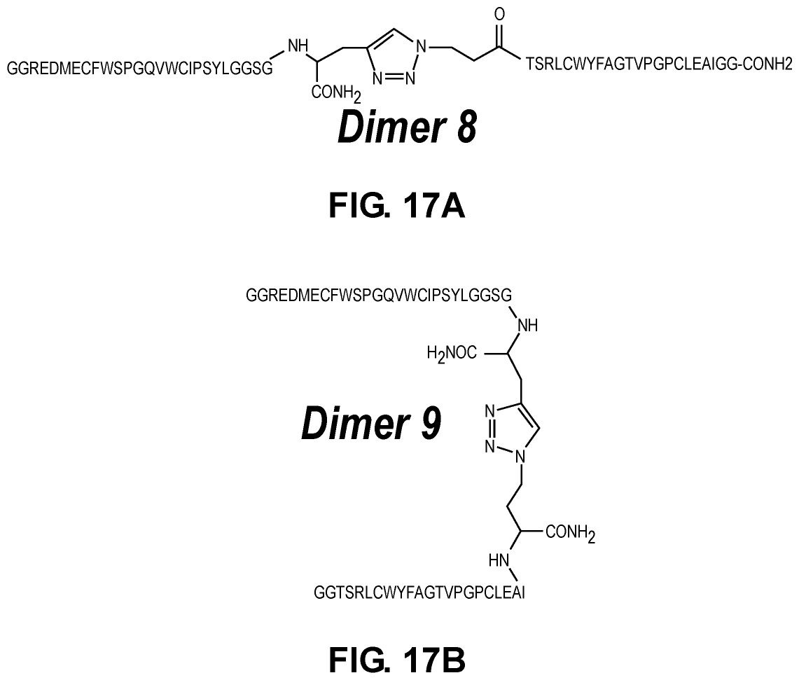

In another instance, an IL-18R binding moiety (dimer 8) comprises an IL-18R alpha binding moiety having the amino acid sequence of GGREDMECFWSPGQVWCIPSYLGGSG-CONH2 (SEQ ID NO: 410) or an amino acid sequence having 1, 2, 3, or 4 substitution(s), addition(s), and/or deletion(s) relative to GGREDMECFWSPGQVWCIPSYLGGSG-CONH2 (SEQ ID NO: 410), and an IL-18R beta binding moiety having the amino acid sequence of TSRLCWYFAGTVPGPCLEAIGG-CONH2 (SEQ ID NO: 411) or an amino acid sequence having 1, 2, 3, or 4 substitution(s), addition(s), and/or deletion(s) relative to TSRLCWYFAGTVPGPCLEAIGG-CONH2 (SEQ ID NO: 411). Such dimers can, optionally, further include a linker and be in any orientation such as N-N, C-C, C-N, or N-C. FIG. 17A provides a representative example of dimer 8 in a C-N configuration.

In another instance, an IL-18R binding moiety (dimer 9) comprises an IL-18R alpha binding moiety having the amino acid sequence of GGREDMECFWSPGQVWCIPSYLGGSG (SEQ ID NO: 412) or an amino acid sequence having 1, 2, 3, or 4 substitution(s), addition(s), and/or deletion(s) relative to GGREDMECFWSPGQVWCIPSYLGGSG (SEQ ID NO: 412), and an IL-18R beta binding moiety having the amino acid sequence of GGTSRLCWYFAGTVPGPCLEAI (SEQ ID NO: 409) or an amino acid sequence having 1, 2, 3, or 4 substitution(s), addition(s), and/or deletion(s) relative to GGTSRLCWYFAGTVPGPCLEAI (SEQ ID NO: 409). Such dimers can, optionally, further include a linker and be in any orientation such as N-N, C-C, C-N, or N-C. FIG. 17B provides a representative example of dimer 9 in a C-C configuration.

In another instance, an IL-18R binding moiety (dimer 10) comprises an IL-18R alpha binding moiety having the amino acid sequence of GGFFECFYSPGHEWCIPSGG (SEQ ID NO: 126) or an amino acid sequence having 1, 2, 3, or 4 substitution(s), addition(s), and/or deletion(s) relative to GGFFECFYSPGHEWCIPSGG (SEQ ID NO: 126), and an IL-18R beta binding moiety having the amino acid sequence of GGLCWYFGGTVRGPCL (SEQ ID NO: 418) or an amino acid sequence having 1, 2, 3, or 4 substitution(s), addition(s), and/or deletion(s) relative to GGLCWYFGGTVRGPCL (SEQ ID NO: 418). In another instance, an IL-18R binding moiety (dimer 10) comprises an IL-18R alpha binding moiety having the amino acid sequence of FFECFYSPGHEWCIPS (SEQ ID NO: 127) or an amino acid sequence having 1, 2, 3, or 4 substitution(s), addition(s), and/or deletion(s) relative to FFECFYSPGHEWCIPS (SEQ ID NO: 127), and an IL-18R beta binding moiety having the amino acid sequence of LCWYFGGTVRGPCL (SEQ ID NO: 218) or an amino acid sequence having 1, 2, 3, or 4 substitution(s), addition(s), and/or deletion(s) relative to LCWYFGGTVRGPCL (SEQ ID NO: 218). Such dimers can, optionally, further include a linker and be in any orientation such as N-N, C-C, C-N, or N-C. FIG. 16D provides a representative example of dimer 10 in a N-C configuration with a linker.

In another instance, an antagonistic IL-18R binding moiety (dimer 11) comprises a first IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and a second IL-18R alpha binding moiety having the amino acid sequence of REDMECFWSPGQVWCIPSYLGG (SEQ ID NO: 162) or an amino acid sequence having 1, 2, 3, or 4 substitution(s), addition(s), and/or deletion(s) relative to REDMECFWSPGQVWCIPSYLGG (SEQ ID NO: 162). Such dimers can, optionally, further include a linker and be in any orientation such as N-N, C-C, C-N, or N-C. FIG. 16E provides a representative example of dimer 11 in a N-N configuration with a linker.

In another instance, an antagonistic IL-18R binding moiety (dimer 12) comprises an IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and a peptide having the amino acid sequence of FEWTPGYWQPYALPLL (SEQ ID NO: 483) or an amino acid sequence having 1, 2, 3, or 4 substitution(s), addition(s), and/or deletion(s) relative to FEWTPGYWQPYALPLL (SEQ ID NO: 483). Such dimers can, optionally, further include a linker and be in any orientation such as N-N, C-C, C-N, or N-C. FIG. 16F provides a representative example of dimer 12 in a N-N configuration with a linker.

In another instance, an antagonistic IL-18R binding moiety (dimer 13) comprises a first IL-18R alpha binding moiety having the amino acid sequence of GGREDMECFWSPGQVWCIPSYLGG-CONH2 (SEQ ID NO: 403) or an amino acid sequence having 1, 2, 3, or 4 substitution(s), addition(s), and/or deletion(s) relative to GGREDMECFWSPGQVWCIPSYLGG-CONH2 (SEQ ID NO: 403), and a second IL-18R alpha binding moiety having the amino acid sequence of YLDTCWVPFHYVNYLMCNVEGG (SEQ ID NO: 33) or an amino acid sequence having 1, 2, 3, or 4 substitution(s), addition(s), and/or deletion(s) relative to YLDTCWVPFHYVNYLMCNVEGG (SEQ ID NO: 480). Such dimers can, optionally, further include a linker and be in any orientation such as N-N, C-C, C-N, or N-C. FIG. 16G provides a representative example of dimer 13 in a N-N configuration with a linker.

It will be understood that, for any peptide described herein having two cysteine (C) residues in the sequence, the cysteine residues may be bridged by a disulfide bond. In one instance, in a molecule comprising both (1) a first peptide sequence having two cysteine residues and (2) a second peptide having two cysteine residues, the first peptide may include a first intrastrand disulfide linkage and the second peptide may include a second intrastrand disulfide linkage. In another instance, in a molecule comprising both (1) a first peptide sequence having two cysteine residues and (2) a second peptide having two cysteine residues, a first cysteine residue on the first peptide may form an interstrand disulfide bridge with a first or second cysteine residue on the second peptide and/or a second cysteine residue on the first peptide may form an interstrand disulfide bridge with a first or second cysteine residue on the second peptide.

### Pharmaceutical Formulations

In another aspect, provided herein is a pharmaceutical formulation comprising (i) one or more of the polypeptide(s) described herein and (ii) a pharmaceutically acceptable excipient. “Pharmaceutically acceptable” refers to approved or approvable by a regulatory agency of the Federal or a state government or listed in the U.S. Pharmacopeia (U.S.P.) or other generally recognized pharmacopeia for use in animals, including humans. A “pharmaceutically acceptable excipient” refers to an excipient that can be administered to a subject and which does not destroy the pharmacological activity of an active agent and is nontoxic when administered in doses sufficient to deliver a therapeutic amount of the agent. A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n—COOH where n is 0-4, and the like. Similarly, pharmaceutically acceptable cations include, but are not limited to, sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

### Methods of Treatment

In another aspect, provided herein is a method of treating a disease or disorder in a subject in need thereof, comprising administering to the subject (a) one or more of the polypeptide(s) described herein or (b) a pharmaceutical formulation. Examples of diseases and disorders to be treated with the described methods include, but are not limited to, a cancer, an autoimmune disease, an inflammatory disease or disorder, an infectious disease or disorder, a metabolic disease or disorder, a neurodegenerative disease or disorder, a myocardial infarction, emphysema, psoriasis, a hemophagocytic syndrome, a macrophage activation syndrome, sepsis, acute kidney injury, or a combination thereof The term “subject” generally refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

A further example of a disease or disorder to be treated with the described methods include, but are not limited to, an Inflammatory Bowel Disease (IBD). In certain embodiments, the polypeptide(s) described herein can be selectively administered or applied to cells, for example, of the intestinal tract or gut of a subject. In certain embodiments, the polypeptide(s) described herein can be selectively administered or applied to cells, for example, of the intestinal tract or gut of a subject in whom IL-18 is highly expressed or overexpressed in the gastrointestinal tract. In certain embodiments, the polypeptide(s) described herein can be administered subcutaneously in a subject with an Inflammatory Bowel Disease or in whom IL-18 is highly expressed or overexpressed in the gastrointestinal tract. In some embodiments, selective administration of the polypeptide(s) described herein effect local inhibition of IL-18 in an organ, for example, the gut or gastrointestinal tract, of a subject. In further embodiments, the selective administration of the polypeptide(s) described herein can act antagonistically to block IL-18 signaling in the cells of the intestinal tract of a subject and preserve the anti-inflammatory function of the IL-37 pathway. In some embodiments, the selective therapeutic administration of the polypeptide(s) described herein can block excess IL-18 signaling and preserve mature goblet cell function for mucin production and restore barrier function in the intestinal tract of a subject with the Inflammatory Bowel Disease or a subject in whom IL-18 is highly expressed or overexpressed in the gastrointestinal tract. In some other embodiments, selective application of the polypeptide(s) described herein can behave as agonists in the cells of a subject.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

“Agonism” (e.g., of an IL-18R binding moiety) is determined using the assay as described in Example 1. An IL-18R agonist is a molecule or binding moiety that has an EC50 value of less than 10 μM as determined in an assay as described in Example 1. In some embodiments, the EC50 value of an IL-18R binding moiety agonist is less than 10 μM, less than 1 μM, less than 300 nM, less than 100 nM, or less than 10 nM. IC50 values based on binding to IL-18R (or its components) are assessed as described in Example 2, while IC50 values based on binding to IL-18BP are assessed as described in Example 4.

“Antagonism” (e.g., of an IL-18R binding moiety) can be determined using an assay as described in Example 5 or 6. An IL-18R antagonist is a molecule or binding moiety that has an EC50 value of less than 10 μM as determined in an assay as described in Example 5 or 6. In some embodiments, the EC50 value of an IL-18R binding moiety agonist is less than 10 uM, less than 1 uM, less than 300 nM, less than 100 nM, or less than 10 nM. An IL-18R antagonist is a molecule or binding moiety that has an IC50 value of less than 10 uM as determined in an assay as described in Example 4. In some embodiments, the IC50 value of an IL-18R binding moiety agonist is less than 10 uM, less than 1 uM, less than 300 nM, less than 100 nM, or less than 10 nM.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “optional” or “optionally” denotes that a subsequently described event or circumstance can but need not occur, and that the description includes instances where the event or circumstance occurs and instances in which it does not.

Amino acids having a large hydrophobic side chain include isoleucine (I), leucine (L), methionine (M), valine (V), phenylalanine (F), tyrosine (Y), and tryptophan (W). Amino acids having a small hydrophobic side chain include alanine (A), glycine (G), proline (P), serine (S), and threonine (T). Amino acids having a basic side chain include arginine (R), lysine (K), and histidine (H). Amino acids having an acidic side chain include aspartate (D) and glutamate (E). Amino acids having a polar/neutral side chain include histidine (H), asparagine (N), glutamine (Q), serine (S), threonine (T), and tyrosine (Y). Amino acids having an aromatic side chain include phenylalanine (F), histidine (H), tryptophan (W), and tyrosine (Y). Amino acids having a hydroxyl (“OH”) side chain include serine (S), threonine (T), and tyrosine (Y). The term “amino acid” includes both naturally occurring and non-naturally occurring amino acids.

A wild-type, human IL-18 refers to an amino acid sequence of, for example,

(UniProtKB A0A024R3E0; SEQ ID NO: 413)
MAAEPVEDNCINFVAMKFIDNTLYFIENLESDYFGKLESK
LSVIRNLNDQVLFIDQGNRPLFEDMTDSDCRDNAPRTIFI
ISMYKDSQPRGMAVTISVKCEKISTLSCENKIISFKEMNP
PDNIKDTKSDIIFFQRSVPGHDNKMQFESSSYEGYFLACE
KERDLFKLILKKEDELGDRSIMFTVQNED.

A human IL-18 binding protein (IL-18BP) refers to an amino acid sequence of, for example,

(UniProtKB 095998; SEQ ID NO: 414)
MTMRHNWTPDLSPLWVLLLCAHVVTLLVRATPVSQTTTAA
TASVRSTKDPCPSQPPVFPAAKQCPALEVTWPEVEVPLNG
TLSLSCVACSRFPNFSILYWLGNGSFIEHLPGRLWEGSTS
RERGSTGTQLCKALVLEQLTPALHSTNFSCVLVDPEQVVQ
RHVVLAQLWAGLRATLPPTQEALPSSHSSPQQQG.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: HEK-BLUE™ IL-18R Reporter Assay

The HEK-BLUE™ IL-18 reporter cell line (InvivoGen #hkb-hmil 18, California, USA) was used to measure the agonist activity of heterodimer peptides composed of IL-18Rα and IL-18Rβ binding peptides. The cell line was generated by stable transfection of HEK293 cells with the genes encoding the alpha and beta subunits of human IL-18 receptor. The cells also express an NF-κB/AP-1-inducible secreted embryonic alkaline phosphatase (SEAP) reporter gene. The binding of IL-18 to the heterodimeric IL-18 receptor triggers a signaling cascade leading to activation of NF-κB and subsequent production of SEAP. Levels of SEAP in the supernatant can be quantified and correspond to the level of receptor activation in response to IL-18 or test compounds.

Cells were maintained and sub-cultured in growth medium (DMEM (4.5 g/L glucose, 2 mM L-glutamine (Corning #10-017-CV)) containing 10% Fetal Bovine Serum (FBS) (Corning #35-015-CV) (heat-inactivated for 30 min at 56° C.), 1× Penicillin Streptomycin (P/S) (Corning #30-002-CI), 1× Normocin™ (InvivoGen #ant-nr-1), supplemented with HEK Blue Selection Antibiotic (1:250; InvivoGen #hb-sel). HEK-BLUE™ IL-18 reporter cells were adherent and seeded at 30 percent confluency in growth medium and harvested for use or split when at 80 to 90 percent confluency. Flasks were incubated in a humidified incubator containing 5% CO₂ at 37° C.

To test compounds for agonist activity HEK-BLUE™ IL-18 reporter cells were harvested by gently rinsing cells twice with pre-warmed PBS (Corning, #21-040-CM) then detaching cells by tapping the flask and gently mixing. The cells were resuspended in fresh, prewarmed test medium (DMEM (4.5 g/L glucose, 2 mM L-glutamine) containing 10% FBS (heat-inactivated) and 1×PBS at approximately 3.0E5 cells per ml. Reporter cells were seeded into wells in a 96 well plate (180 μl per well; 5.0E4 cells). Dilutions of test compounds and IL-18 were prepared in test medium in a v-bottom polypropylene 96-well plate. The starting concentration of test compounds varied depending on predicted potency from 0.1-5 μM. Human recombinant IL-18 (R&D Systems #9124-IL-050/CF) was routinely tested at dilutions starting at 50 pM. Twenty (20) μl of the test compound and IL-18 dilutions were added to the 96-well flat-bottom assay plate containing seeded reporter cells. The plate was incubated at 37° C. in a 5% CO₂ incubator for 20 hr.

The following day, QUANTI-Blue™ Solution (InvivoGen #rep-qbs) was prepared by adding 1 ml of QUANTI-Blue reagent and 1 ml QUANTI-Blue buffer to 98 ml of sterile water. 180 μl of QUANTI-Blue™ Solution was added to wells of a new flat-bottom 96-well plate. Then 20 μl (1:10 final dilution) of the reporter cell supernatant was added to the QUANTI-Blue™ Solution and incubated for 2 hr at 37° C. Absorbance signal at 630 nm was then measured on a plate reader (Agilent BioTek Synergy HTX Multimode Reader). Half maximal effective concentration (EC50) was calculated based on a variable slope and four parameter analysis using GraphPad Prism software.

Dimers 1, 2, and 3 were found to have a potency (EC50) of less than 1 μM, greater than 100 nM and less than 300 nM, and less than 10 nM, respectively.

| | # IL-18R | # IL-18R | | | |
|---|---|---|---|---|---|
| | alpha | beta | | | |
| | binding | binding | | | |
| # dimer | moiety | moiety | linker | potency | efficacy* |
| Dimer 1 | ICWVPFHY | GGCPNWLS | (PA)8 | <1 μM | 17% |
| | LDELMCTGG | DHLSEIWC | (SEQ | | |
| | (SEQ ID | (SEQ ID | ID | | |
| | NO: 415) | NO: 416) | NO: | | |
| | | | 401) | | |
| Dimer 2 | FFECFYSPG | GGCPNWLS | (PA)8 | >100 nM | 34% |
| | HEWCIPSGG | DHLSEIWC | (SEQ | and | |
| | (SEQ ID | (SEQ ID | ID | less | |
| | NO: 417) | NO: 416) | NO: | than | |
| | | | 401) | 300 nM | |
| Dimer 3 | FFECFYSPG | GGLCWYFG | (PA)8 | <30 nM | 36% |
| | HEWCIPSGG | GTVRGPCL | (SEQ | | |
| | (SEQ ID | (SEQ ID | ID | | |
| | NO: 417) | NO: 418) | NO: | | |
| | | | 401) | | |

*% maximum signal (Emax) vs. IL-18 in reporter assay.

### Example 2: Competitive Binding to the IL-18Rα Subunit and to the IL-1812β Subunit

Binding of synthetic peptide ligands to IL-18Rα and IL-18Rβ was evaluated using a competition ELISA.

For IL-18Rα ligands, microtiter plate wells were first coated with IL-18Rα-Fc (CD218a protein, Fc tag; ECD 19-329; ACROBiosystems, Inc, Cat. No. IL1-H5259) at 1 μg/mL; 50 μL per well in PBS for at least 1 hr. The plate was washed with wash buffer (300 μL, PBS containing 0.05% TWEEN®-20 (Sigma)) and then blocked with PBS containing 1% BSA (BSA Fraction V; VWR Cat. No. 97061-416) for 1 hr. A serial dilution of the peptides was prepared, at twice the final concentration, in assay buffer (PBS containing 0.5% BSA and 0.05% TWEEN®-20) in a 96-well polypropylene plate. A C-terminal biotinylated form of the reference IL-18Rα peptide ligand having the amino acid sequence of GGELCWRPFSFVDCEEGG (SEQ ID NO: 15) or GGWSQCLWEPLWECWTDGG (SEQ ID NO: 125) was used to make a pre-complexed tracer with NeutrAvidin-HRP (NA-HRP; ThermoFisher Cat. No. 31030) (Pre-complexed tracer will be referred to as bnPeptide/NA-HRP tracer). The bnPeptide/NA-HRP tracer was prepared by mixing 1.5 μL 100 μM biotinylated peptide, 2 μL NA-HRP and 11.5 μL PBS and incubating at 4° C. for at least 45 min. After blocking the wells, the plate was washed with a plate washer and serial dilutions of the peptides were added (50 μL/well) and the plate was incubated at 4° C. for 1 hr on a plate shaker. The bnPeptide/NA-HRP tracer was diluted to twice its binding EC50-EC75 and 50 μL was added to each assay well. The plate was returned to 4° C. and incubated for 45 min. Following this incubation, the plate was washed using a plate washer and cold wash buffer. Fifty (50) μL of TMB One Component HRP Microwell substrate (TMB; Surmodics Cat. No. TMBW-1000-01) was added to each well, and the plate was incubated for 5-15 min at 25° C. Fifty (50) μL of a stop solution (Surmodics Cat. No. LSTP-0100-0) were added to each well. ELISA signal was then measured at 450 nm in a plate reader (Wallac Victor2 1420 Multilabel counter). The half maximal inhibition concentration (IC50) was calculated based on a variable slope and four parameter analysis using GraphPad Prism software.

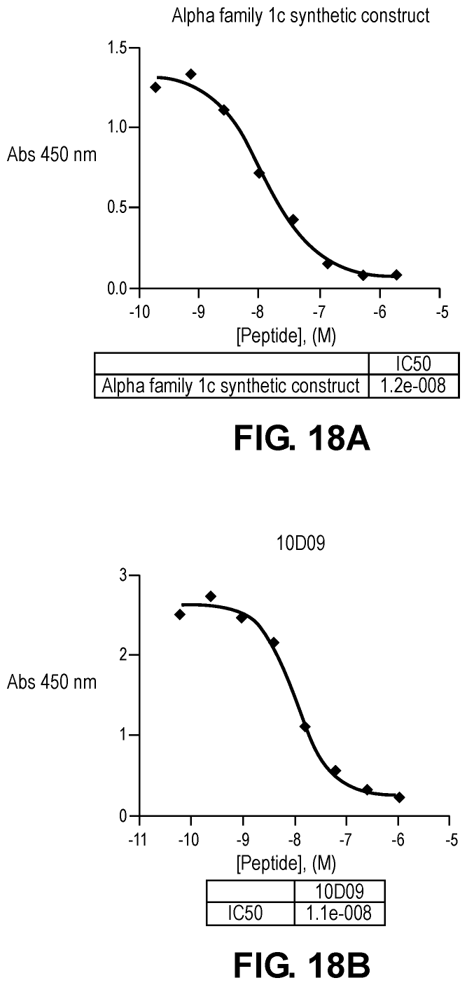

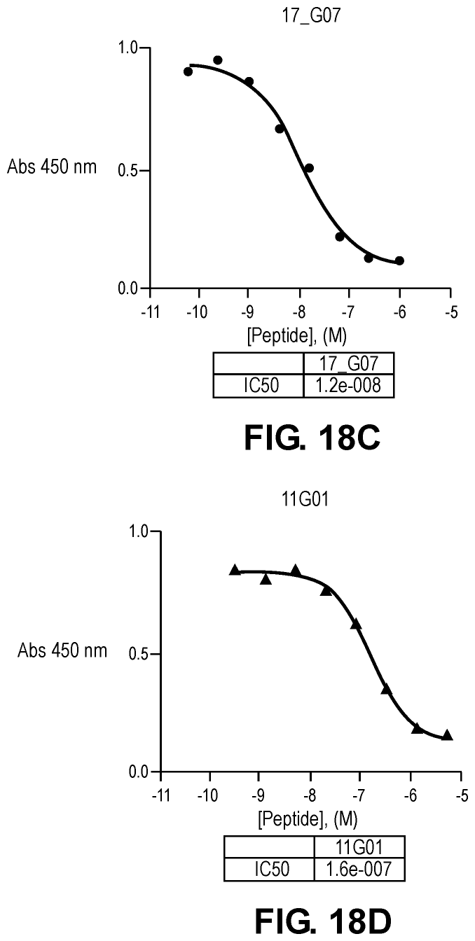

A similar method was used to assess binding of IL-18Rβ peptide ligands to the IL-18Rβ subunit. Microtiter plate wells were coated with IL-18Rβ-Fc (CD218ab protein, Fc tag; ECD 1-357; Sino Biological, Inc., Cat. No. 10176-H02H). As described above, a C-terminal biotinylated form of the reference IL-18Rβ peptide ligand having the amino acid sequence of GGLCWYFGGTVRGPCLGG (SEQ ID NO: 217) or GGCPNWLSDHLSEIWCGG (SEQ ID NO: 341) was used to make a pre-complexed tracer with NeutrAvidin-HRP. All other steps are the same as described above. The results for assessing binding of IL-18Rb and IL-18Ra peptide ligands to their respective IL-18R subunits are presented in FIGS. 18A-D and in the table below.

An alternative competition binding assay was used in some cases to evaluate binding of peptide ligands to the IL-18 receptor subunits and to determine whether an IL-18R binding protein is antagonistic or agonistic. The method was the same as described above except 20-50 μL of culture supernatant containing representative peptide-display phage were used as the tracer. After the 45 min incubation with peptide ligands and phage supernatants, the plates were washed and bound phage were detected with 50 μL of anti-M13 antibody HRP conjugate (Sino Biological, Inc. Cat. No.11973-MM05T-H) diluted 5000-fold into assay buffer. The plate was incubated at 4° C. for 1 hr. and then washed using the plate washer and cold wash buffer. Fifty (50) μL of TMB One Component HRP Microwell substrate was added to each well and the plate was incubated for 5-15 min at 25° C. Fifty (50) μL of stop solution were added to each well. ELISA signal was then measured at 450 nm in a plate reader and the half maximal inhibition concentration (IC50) was calculated based on a variable slope and four parameter analysis using GraphPad Prism software. Each molecule tested was determined to have an IC50 of less than 10 μM.

IC50 data for representative examples of clones is as follows:

| Binding moiety | Clone | IC50 |
|---|---|---|
| IL-18R alpha binding moieties | 4D07 | 1.2e−008 |
| | 7C06 | 3.5e−007 |
| IL-18R beta binding moieties | Family 2 synthetic construct | 6.7e−008 |
| | 5C09 | 3.2e−007 |

### Example 3: Binding Affinity Assay for IL-18BP.

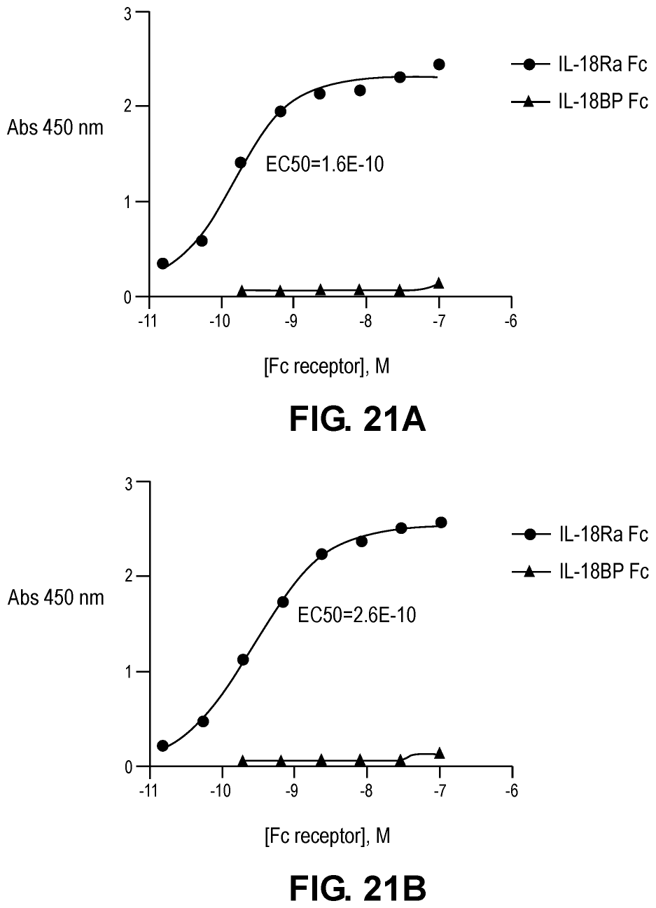

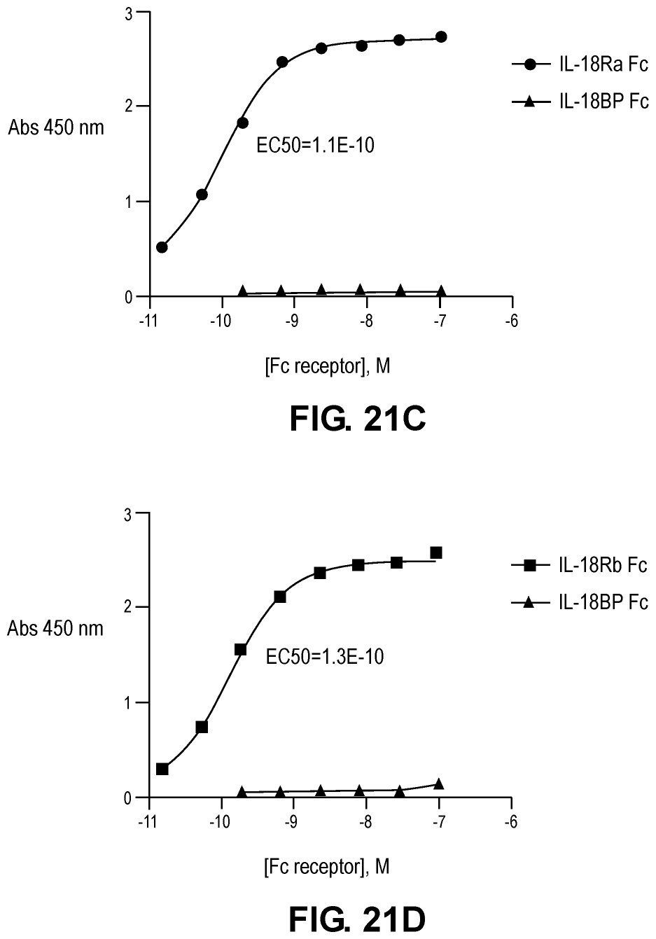

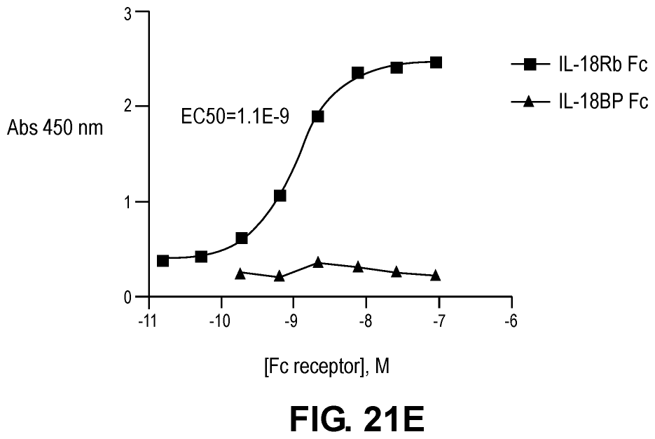

Binding of synthetic monomer peptides to IL-18 Binding Protein (IL-18BP) was measured using a direct binding ELISA. Briefly, 96-well ELISA plates were coated with NeutrAvidin (NA) at 5 μg per well in PBS, washed, then blocked with PBS containing 1% BSA. Wells were washed and representative peptides, synthesized with a C-terminal biotin were diluted to 300 nM in assay buffer and added to the NA coated wells. Wells were washed and serial dilutions starting at 100 nM of IL-18R subunits, IL-18BP from either of two sources (Acro #ILP-H5253 and R&D #119-BP), and control IgG Fc fusions) were added to the wells and incubated for 1 hr at 4° C. Then plates were washed and goat anti-human IgG Fc-HRP (Southern Biotech #2048-05) detection antibody was added for 1 hr. Wells were developed with TMB for 5-15 min and the enzyme reaction was stopped with a H₂SO₄ solution. The C-terminally biotinylated forms of peptides 4D07, 7C06, alpha Synthetic Fla Consensus Construct, beta family 2 synthetic construct, and 5C09 did not exhibit binding to IL-18BP at up to 100 nM IL-18BP. The results are presented in FIGS. 21A-E, respectively.

### Example 4: HEK-BLUE™ IL18R Reporter Assay for IL-18BP/IL-18 Interaction

IL-18 is negatively regulated by a decoy receptor called IL-18 binding protein (IL-18BP), a secreted antagonist that binds IL-18 with extremely high affinity. HEK-BLUE™ IL-18 reporter cell line (InvivoGen #hkb-hmil 18, California, USA) was used to determine the impact of heterodimer peptides composed of IL-18Ra and IL-18Rb binding peptides on the IL-18BP/IL-18 interaction. The IL-18 Reporter assay, as described in detail in Example 2, was used to investigate this interaction with the following modifications.

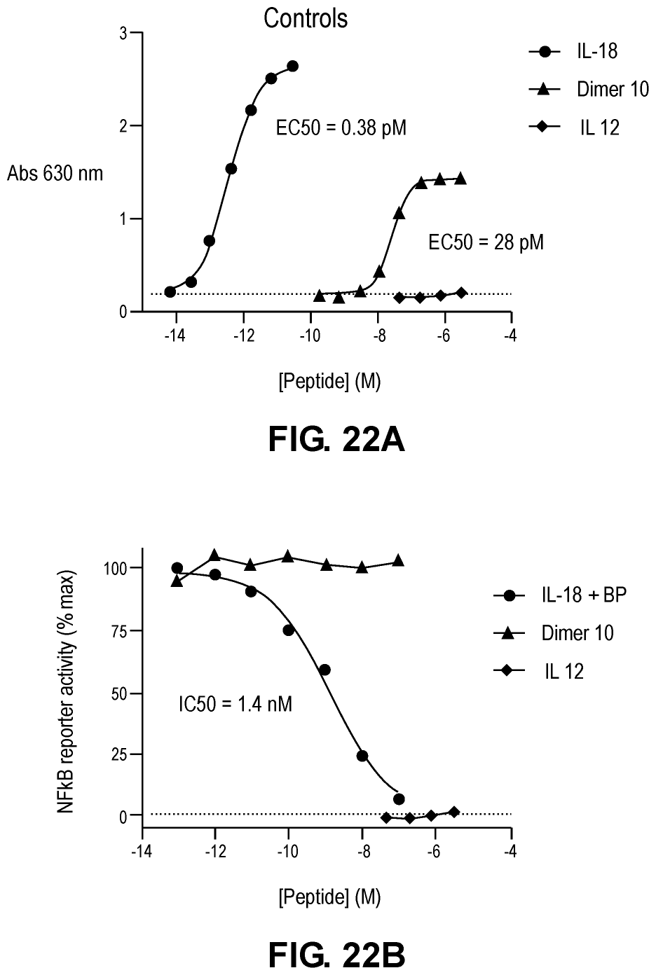

A serial dilution (7-point, 10-fold) of IL-18BP Fc fusion (Acro #ILP-H5253) starting at 2 μM (20-fold final concentration) was prepared in test medium in a v-bottom polypropylene 96-well plate. Human recombinant IL-18 (R&D Systems #9124-IL-050/CF) and a representative peptide agonist were prepared at 30 pM and 600 nM respectively (both at 20-fold final concentration and at approximately their respective EC70s). Equal volumes of the IL-18BP dilution and 30 pM IL-18 were premixed in a 96-well polypropylene plate for 30 min. The same was done with the IL-18BP dilution and 600 nM peptide agonist. Meanwhile, HEK-BLUE™ IL-18 reporter cells were harvested and resuspended at approximately 3.0E5 cells per mL, as previously described. Reporter cells were seeded into wells in a 96 well plate (180 μl per well; 5.0E4 cells). After the 30 min pre-incubation of IL-18BP with IL-18 or peptide agonist, 20 μl of the mixtures were added to assay plate containing seeded reporter cells. The plate was incubated at 37° C. in a 5% CO₂ incubator for 20 hr. The following day, QUANTI-BLUE™ Solution was prepared as previously described. 180 μl of QUANTI-BLUE™ Solution was added to wells of a new flat-bottom 96-well plate. Then 20 μl (1:10 final dilution) of the reporter cell supernatant was added to the QUANTI-BLUE™ Solution and incubated for 2 hr at 37° C. Absorbance signal at 630 nm was then measured on a plate reader. Half maximal inhibition concentration (IC50) was calculated based on a variable slope and four parameter analysis using GraphPad Prism software. When no inhibition was measured the data points were connected without a curve fit. Absorbance readings were converted to percent total of signal span, for IL-18 and peptide agonist respectively, and graphed as NF-κB reporter activity (% max). As shown in FIG. 22, IL-18 and dimer 10 are agonists of IL-18R with EC5Os of 0.38 pM and 28 nM, respectively (FIG. 22A). Further, the IL-18BP was confirmed to inhibit bioactivity of IL-18 with an IC50 of ˜1nM, and IL-18BP does not inhibit the agonist activity of dimer 10 up to 100 nM (FIG. 22B).

### Example 5: Inhibition of IFNg Secretion in KG-1 Cells by IL-18R Binding Peptides

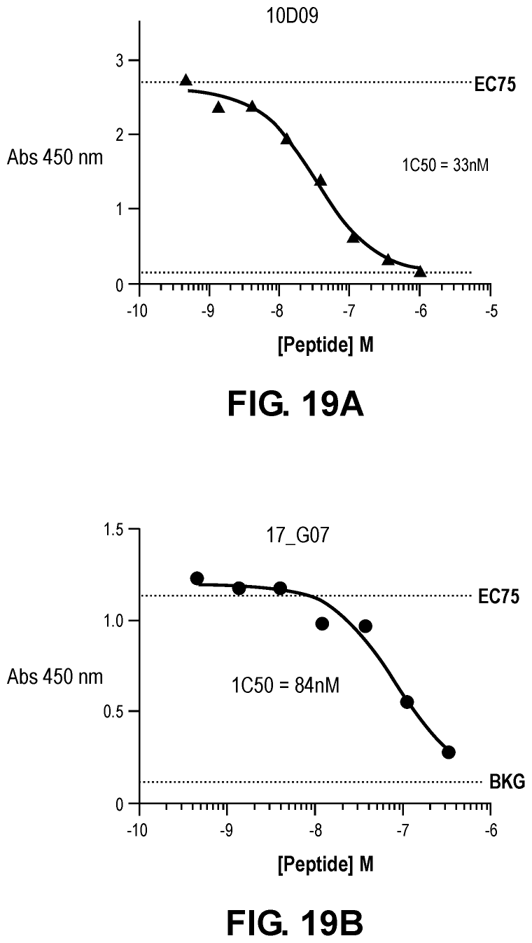

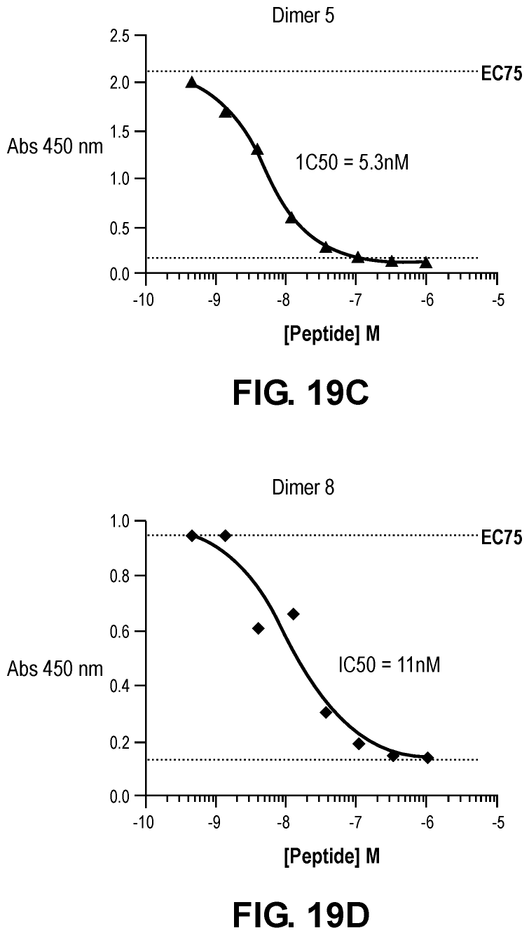

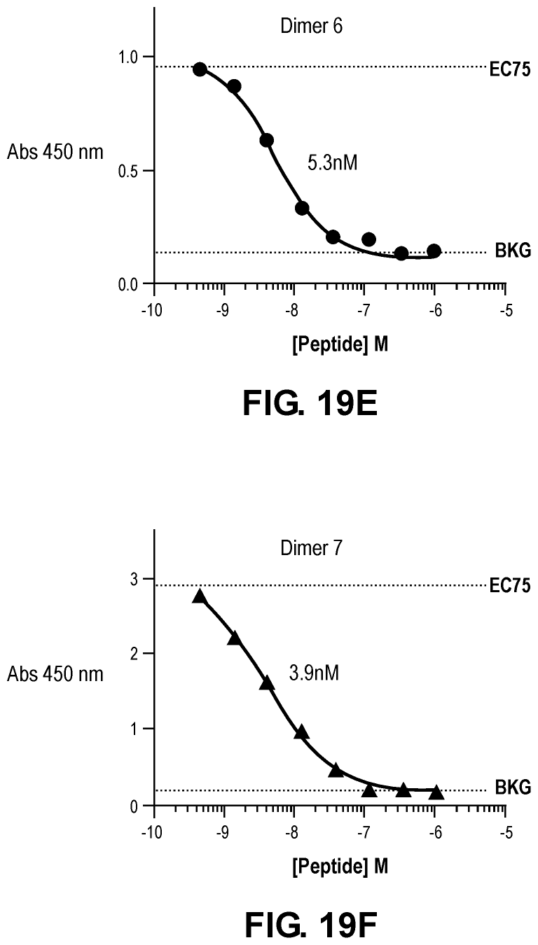

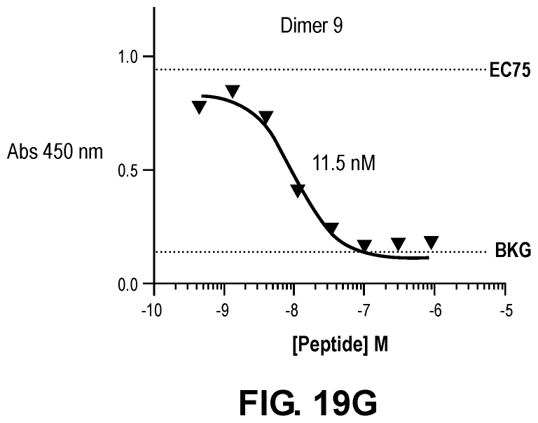

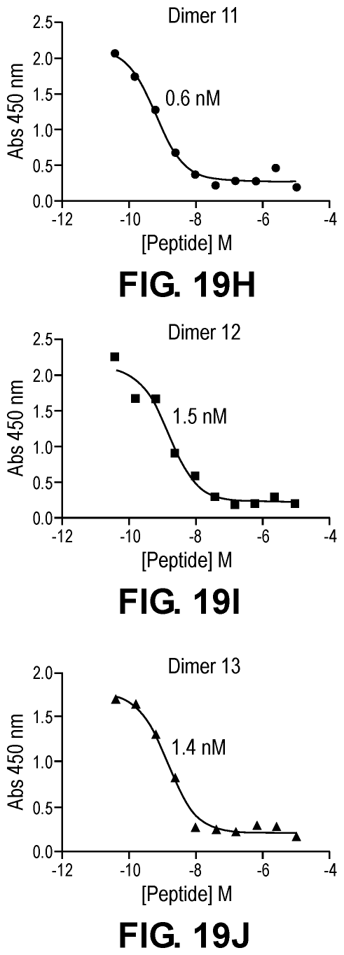

IL-18R-binding peptides were evaluated for inhibition of IFNg secretion in KG-1 cells stimulated with IL-18. The human myeloid leukemia cell line KG-1 was obtained from ATCC (CCL-246). KG-1 cells were maintained in culture medium (IMDM+20% FBS+1% P/S). To test compounds for antagonist activity, cells were resuspended in culture medium at 1.5×10⁶ cells/ml and plated in 96-well culture plates at 3×10⁵ cells/well. Three-fold serial dilutions of IL-18R ligands in culture medium were added to the wells and incubated for 10-20 min at 37° C. IL-18 (R&D Systems #9124-IL-010) was then added to each well at a final concentration 2 ng/ml (EC75), and the plates were incubated for an additional 24 h at 37° C. in a 5% CO₂ incubator. Plates were centrifuged at 900 rpm for 5 minutes to pellet the cells and supernatants were transferred to a 96-well plate. The amount of IFNγ present in each sample was quantified by sandwich ELISA (BD Biosciences #555142) according to the manufacturer's instructions. Cell supernatants (100 ml) were added to microwells that were pre-coated with an anti-human IFNg monoclonal antibody and incubated for 2 h at RT. Wells were then washed with PBS 0.05% Tween-20 (PBST) and bound IFNg was detected by adding a biotinylated anti-human IFNg detection antibody plus streptavidin HRP conjugate and incubating for 1 h at RT. Wells were washed with PBST and TMB substrate solution was added to measure the amount of HRP in each well. Absorbance at 450 nm was read in a microplate reader (Agilent BioTek Synergy HTX Multimode Reader). The signal produced is proportional to the quantity of IFNg present in each cell supernatant. The results are presented in FIGS. 19A-G.

### Example 6: Inhibition of IFNg Secretion in Human NK cells by IL-18 Binding Peptides

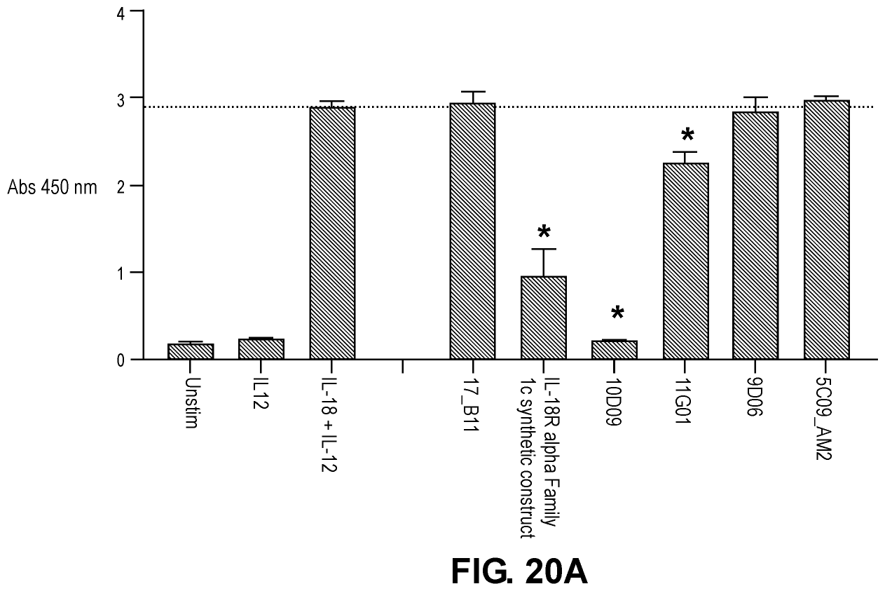

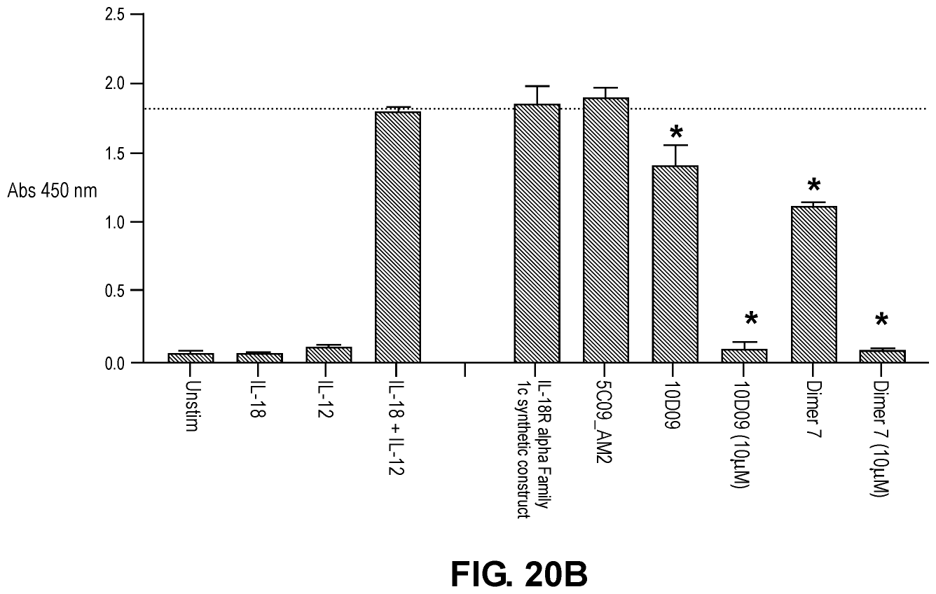

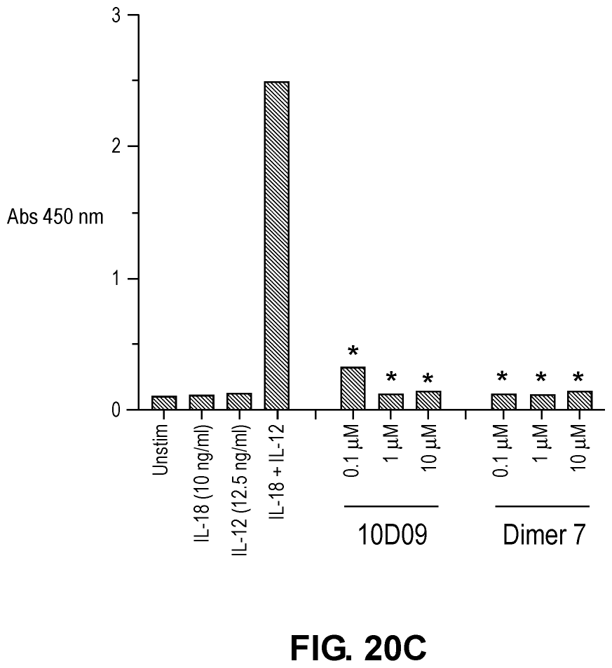

IL-18R-binding peptides were evaluated for inhibition of IFNg secretion in primary human NK cells co-stimulated with IL-12+IL-18. Cryopreserved human NK cells isolated from PBMCs using immunomagnetic negative selection were obtained from the Stanford Blood Center (Palo Alto, CA). To test compounds for antagonist activity, frozen NK cells were thawed, washed several times with culture medium (EMEM+12.5% horse serum+12.5% FBS+0.2 mM inositol+1 mM L-glutamine+1% P/S), and resuspended at 1×10⁶ cells/ml. Cells were plated in 96-well culture plates at 7.5×10⁴ cells/well. IL-18R binding peptides diluted in culture medium were added to the wells and incubated for 30 min at 37° C. IL-12 (Peprotech #200-12) and IL-18 (R&D Systems #9124-IL-010) were then added to each well at a final concentration of 12.5 ng/ml and 10 ng/ml respectively, and the plates were incubated for an additional 8 h at 37° C. in a 5% CO₂ incubator. Plates were centrifuged at 900 rpm for 5 minutes to pellet the cells, and supernatants were transferred to a 96-well plate. The amount of IFNγ present in each sample was quantified by sandwich ELISA (BD Biosciences #555142) according to the manufacturer's instructions. Cell supernatants (100 ml) were added to microwells that were pre-coated with an anti-human IFNg monoclonal antibody and incubated for 2 h at RT. Plates were then washed with PBS 0.05% Tween-20 (PBST) and bound IFNg was detected by adding a biotinylated anti-human IFNg detection antibody plus streptavidin HRP conjugate and incubating for 1 h at RT. Plates were washed with PBST and TMB substrate solution (Surmodics #TMBW-1000-01) was added to measure the amount of HRP in each well. An equal volume of stop solution (Surmodics #LSTP-0100-01) was added to each well and the absorbance at 450 nm was measured in a microplate reader (Agilent BioTek Synergy HTX Multimode Reader). The signal produced is proportional to the quantity of IFNg present in each cell supernatant. The results are presented in FIGS. 20A-C.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

1-255. (canceled)

256. A polypeptide comprising an interleukin 18 (IL-18) receptor (IL-18R) binding moiety, wherein the IL-18R binding moiety comprises an IL-18R alpha binding moiety; and wherein the IL-18R alpha binding moiety comprises the amino acid sequence of X261-X262-X263-X264-X265-X266-X267-X268-X269-X270-X271-X272-X273-X274-X275-X276-X277-X278-X279-X280-X281-X282-X283-X284, wherein
X261 is G or absent;
X262 is G or absent;
X263 is any amino acid;
X264 is any amino acid;
X265 is L, M, R, S, T, Y, Q, W, I, V, F, A, G, or P;
X266 is L, M, I, T, S, V, F, Y, W, A, G, or P;
X267 is C;
X268 is F, S, or W;
X269 is R, V, L, M, H, W, F, I, Y, A, G, P, S, or T;
X270 is P;
X271 is W, or F;
X272 is E, P, Q, N, or S;
X273 is D, G, or E;
X274 is V, L, I, T, F, M, Y, W, A, G, P, or S;
X275 is E, A, D, G, F, or M;
X276 is D;
X277 is L, F, H, R, or S;
X278 is V, I, F, L, M, Y, W, A, G, P, S, or T;
X279 is C;
X280 is T, M, I, W, or S;
X281 is E, G, S, T, A, W, D, or Y;
X282 is any amino acid;
X283 is G or absent; and
X284 is G or absent (SEQ ID NO: 476).

257. The polypeptide of claim 256, wherein
X263 is H, E, V, N, L, R, S, T, F, or D;
X264 is D, R, Q, H, Y, S, K, or T; and
X282 is D, V, R, N, Q, T, S, L, or Y.

258. The polypeptide of claim 256, wherein the IL-18R alpha binding moiety comprises (i) the amino acid sequence of
(SEQ ID NO: 53)
GGHDLLCFRPWEDVEDLVCTEDGG;
(SEQ ID NO: 54)
GGERLMCSVPWEDVADFICMGDGG;
(SEQ ID NO: 55)
GGVQMICWLPWPDVDDHICTEVGG;
(SEQ ID NO: 56)
GGNHRICWLPWEDVDDRICISRGG;
(SEQ ID NO: 57)
GGLYSMCFRPWEDVEDFICTTNGG;
(SEQ ID NO: 58)
GGRSTLCWVPWEGLGDRICTAQGG;
(SEQ ID NO: 59)
GGSYLTCWVPWQGLDDRFCTETGG;
(SEQ ID NO: 60)
GGTDYTCFVPWNDVFDRVCTWSGG;
(SEQ ID NO: 61)
GGSHLICWMPWPEVEDRLCTDSGG;
(SEQ ID NO: 62)
GGTKLMCWVPWQDVDDFICTSLGG;
(SEQ ID NO: 63)
GGFYQSCWHPWEDIDDHICTYSGG;
(SEQ ID NO: 64)
GGFKLSCWVPWQDTEDRICWTVGG;
(SEQ ID NO: 65)
GGSHWICWWPFSDTEDHICSEYGG;
(SEQ ID NO: 66)
GGSRLVCWVPFEDFMDSICTEYGG;
(SEQ ID NO: 67)
GGDTYTCFFPWPGLDDHFCTYSGG;
or
(ii) an amino acid sequence having 1, 2, 3, or 4 substitution(s), addition(s), and/or deletion(s) relative to the amino acid sequence of (i).

259. The polypeptide of claim 256, wherein X261, X262, X283, and X284 are absent and the IL-18R alpha binding moiety comprises (i) the amino acid sequence of
(SEQ ID NO: 68)
HDLLCFRPWEDVEDLVCTED;
(SEQ ID NO: 69)
ERLMCSVPWEDVADFICMGD;
(SEQ ID NO: 70)
VQMICWLPWPDVDDHICTEV;
(SEQ ID NO: 71)
NHRICWLPWEDVDDRICISR;
(SEQ ID NO: 72)
LYSMCFRPWEDVEDFICTTN;
(SEQ ID NO: 73)
RSTLCWVPWEGLGDRICTAQ;
(SEQ ID NO: 74)
SYLTCWVPWQGLDDRFCTET;
(SEQ ID NO: 75)
TDYTCFVPWNDVFDRVCTWS;
(SEQ ID NO: 76)
SHLICWMPWPEVEDRLCTDS;
(SEQ ID NO: 77)
TKLMCWVPWQDVDDFICTSL;
(SEQ ID NO: 78)
FYQSCWHPWEDIDDHICTYS;
(SEQ ID NO: 79)
FKLSCWVPWQDTEDRICWTV;
(SEQ ID NO: 80)
SHWICWWPFSDTEDHICSEY;
(SEQ ID NO: 81)
SRLVCWVPFEDFMDSICTEY;
or
(SEQ ID NO: 82)
DTYTCFFPWPGLDDHFCTYS;
or
(ii) an amino acid sequence having 1, 2, 3, or 4 substitution(s), addition(s), and/or deletion(s) relative to the amino acid sequence of (i).

260. The polypeptide of claim 256, wherein X261 and X262 are absent and the IL-18R alpha binding moiety comprises (i) the amino acid sequence of GGICWVPFHYLDELMCTGG (SEQ ID NO: 2) or an amino acid sequence having 1, 2, 3, or 4 substitution(s), addition(s), and/or deletion(s) relative to the amino acid sequence of SEQ ID NO: 2.

261. A polypeptide comprising an interleukin 18 (IL-18) receptor (IL-18R) binding moiety, wherein the IL-18R binding moiety comprises an IL-18R alpha binding moiety; and wherein the IL-18R alpha binding moiety comprises the amino acid sequence of X61-X62-X63-X64-X65-X66-X67-X68-X69-X70-X71-X72-X73-X74-X75-X76-X77-X78-X79-X80-X81-X82-X83-X84, wherein:
(i) one or more of X61, X62, X63, X64, X65, X80, X81, X82, X83, and X84 are optional,
X61 is G or absent;
X62 is G or absent;
X63 is absent or any amino acid;
X64 is G, N, S, V, D, Y, E, A or absent;
X65 is M, A, P, T, W, F, G, Y, F, S, V, H, D, K, L, I, or absent;
X66 is I, V, Y, A, G, P, T, S, F, M, L, or W;
X67 is Q, E, or D;
X68 is C;
X69 is L, F, W, A, or Y;
X70 is W or Y;
X71 is E or S;
X72 is P;
X73 is L, G, H, T, S, or Y;
X74 is W, H, E, Q, or A;
X75 is E, V, Y, H, or L;
X76 is C or W;
X77 is W or C;
X78 is T, I, S, M, or W;
X79 is D, P, or T;
X80 is G, S, H, I, T, A, or absent;
X81 is M, W, A, P, S, T, G, L, V, F, Y, Q, I, or absent;
X82 is V, F, Y, A, S, G, M, I, T, N, F, D, L, Q, W, P, or absent;
X83 is G or absent; and
X84 is G or absent (SEQ ID NO: 485).

262. The polypeptide of claim 261, wherein
X63 is any amino acid;
X64 is N, D, or E;
X65 is D, I, L, M, V, F, Y, W, A, G, P, S, T, H, or Y;
X66 is I, L, M, V, F, Y, W, A, G, P, S, or T;
X67 is E, or D;
X68 is C;
X69 is F;
X70 is W or Y;
X71 is S;
X72 is P,
X73 is G or S;
X74 is Q;
X75 is E, V, or Y;
X76 is W;
X77 is C;
X78 is I;
X79 is P;
X80 is S;
X81 is I, L, M, V, F, Y, W, A, G, P, S, or T; and
X82 is I, L, M, V, F, Y, W, A, G, P, S, or T (SEQ ID NO: 460).

263. The polypeptide of claim 261, wherein the IL-18R alpha binding moiety comprises (i) the amino acid sequence of
(SEQ ID NO: 97)
GGANYLECFYSPTQEWCIPHLMGG;
(SEQ ID NO: 98)
GGESFLECFYSPAEWCIPSVIGG;
(SEQ ID NO: 99)
GGFVFMECFWSPSQEWCIPSFTGG;
(SEQ ID NO: 100)
GGGVSFDCFWSPGQYWCIPDYNGG;
(SEQ ID NO: 101)
GGLDVMECFWSPSQVWCMPSVFGG;
(SEQ ID NO: 102)
GGLYHFECAWSPYQHWCWPSQDGG;
(SEQ ID NO: 103)
GGREDMECFWSPGQVWCIPSYLGG;
(SEQ ID NO: 104)
GGREKLECFWSPGQEWCIPILQGG;
(SEQ ID NO: 105)
GGRGFLECFYSPGQEWCIPSIWGG;
(SEQ ID NO: 106)
GGSALLECFYSPTQEWCIPTLWGG;
(SEQ ID NO: 107)
GGSSYSECFWSPGQLWCIPSFPGG;
(SEQ ID NO: 108)
GGVSIFECFYSPTQEWCIPSFIGG;
(SEQ ID NO: 109)
GGWDILECFYSPGQEWCIPSFLGG;
or
(SEQ ID NO: 110)
GGYGSWECYWSPSEEWCIPAQLGG;
or
(ii) an amino acid sequence having 1, 2, 3, or 4 substitution(s), addition(s), and/or deletion(s) relative to any one of (i).

264. The polypeptide of claim 261, wherein X61, X62, X83, and X84 are absent; and
wherein the IL-18R alpha binding moiety comprises (i) the amino acid sequence of
(SEQ ID NO: 111)
ANYLECFYSPTQEWCIPHLM;
(SEQ ID NO: 112)
ESFLECFYSPSAEWCIPSVI;
(SEQ ID NO: 113)
FVFMECFWSPSQEWCIPSFT;
(SEQ ID NO: 114)
GVSFDCFWSPGQYWCIPDYN;
(SEQ ID NO: 115)
LDVMECFWSPSQVWCMPSVF;
(SEQ ID NO: 116)
LYHFECAWSPYQHWCWPSQD;
(SEQ ID NO: 117)
REDMECFWSPGQVWCIPSYL;
(SEQ ID NO: 118)
REKLECFWSPGQEWCIPILQ;
(SEQ ID NO: 119)
RGFLECFYSPGQEWCIPSIW;
(SEQ ID NO: 120)
SALLECFYSPTQEWCIPTLW;
(SEQ ID NO: 121)
SSYSECFWSPGQLWCIPSFP;
(SEQ ID NO: 122)
VSIFECFYSPTQEWCIPSFI;
(SEQ ID NO: 123)
WDILECFYSPGQEWCIPSFL;
or
(SEQ ID NO: 124)
YGSWECYWSPSEEWCIPAQL;
or
(ii) an amino acid sequence having 1, 2, 3, or 4 substitution(s), addition(s), and/or deletion(s) relative to any one of (i).

265. The polypeptide of claim 261, wherein X61, X62, X83, and X84 are absent; and wherein the IL-18R alpha binding moiety comprises (i) the amino acid sequence of GGFFECFYSPGHEWCIPSGG (SEQ ID NO: 126)
or (ii) an amino acid sequence having 1, 2, 3, or 4 substitution(s), addition(s), and/or deletion(s) relative to the amino acid sequence of (i).

266. The polypeptide of claim 261, wherein X61, X62, X63, X64, X81, X82, X83, and X84 are absent; and wherein the IL-18R alpha binding moiety comprises (i) the amino acid sequence of FFECFYSPGHEWCIPS (SEQ ID NO: 127) or (ii) an amino acid sequence having 1, 2, 3, or 4 substitution(s), addition(s), and/or deletion(s) relative to the amino acid sequence of (i).

267. The polypeptide of claim 261, wherein X61, X62, X63, X82, X83, and X84 are absent; and wherein the IL-18R alpha binding moiety comprises (i) the amino acid sequence of GGMECWYSPHEVWCSTGG (SEQ ID NO: 128) or (ii) an amino acid sequence having 1, 2, 3, or 4 substitution(s), addition(s), and/or deletion(s) relative to the amino acid sequence of (i).

268. The polypeptide of claim 261, wherein X61, X62, X63, X64, X65, X80, X81, X82, X83, and X84 are absent; and wherein the IL-18R alpha binding moiety comprises (i) the amino acid sequence of MECWYSPHEVWCST (SEQ ID NO: 129) or (ii) an amino acid sequence having 1, 2, 3, or 4 substitution(s), addition(s), and/or deletion(s) relative to the amino acid sequence of (i).

269. A polypeptide comprising an interleukin 18 (IL-18) receptor (IL-18R) binding moiety, wherein the IL-18R binding moiety comprises an IL-18R alpha binding moiety; and wherein the IL-18R alpha binding moiety comprises the amino acid sequence of X105-X106-X107-X108-X109-X110-X111-X112-X113-X114-X115-X116-X117-X118-X119-X120-X121-X122-X123-X124-X125-X126-X127-X128, wherein:
X105 is G or absent;
X106 is G or absent;
X107 is G or Q or absent;
X108 is any amino acid or absent;
X109 is any amino acid;
X110 is L or C;
X111 is H, F, or W;
X112 is C, W, Q, or I;
X113 is F, V, D, or S;
X114 is E, N, Y, D, or L;
X115 is S, D, or P;
X116 is R, M, or G;
X117 is any amino acid;
X118 is any amino acid;
X119 is M, F, Y, W, A, G, P, S, T, D, V, L, or I;
X120 is I, L, M, F, Y, A, G, P, S, T, F, H, W, V, or Y;
X121 is C;
X122 is any amino acid;
X123 is any amino acid or absent;
X124 is any amino acid or absent;
X125 is any amino acid or absent;
X126 is S or absent;
X127 is G or absent; and
X128 is G or absent (SEQ ID NO: 486).

270. The polypeptide of claim 269, wherein
X108 is any amino acid;
X109 is any amino acid;
X110 is L or C;
X111 is H or W;
X112 is C or Q;
X113 is F or D;
X114 is E, N, or D;
X115 is S or P;
X116 is R or G;
X117 is any amino acid;
X118 is any amino acid;
X119 is D, I, L, M, V, F, Y, W, A, G, P, S, or T;
X120 is I, L, M, V, F, Y, W, A, G, P, S, T, or H;
X121 is C;
X122 is any amino acid;
X123 is any amino acid;
X124 is any amino acid; and
X125 is any amino acid (SEQ ID NO: 463).

271. The polypeptide of claim 269, wherein the IL-18R alpha binding moiety comprises (i) the amino acid sequence of GGQTFLHCFESRGIDWCIPWKSGG (SEQ ID NO:

176. or (ii) an amino acid sequence having 1, 2, 3, or 4 substitution(s), addition(s), and/or deletion(s) relative to the amino acid sequence of (i).

272. The polypeptide of claim 269, wherein X105, X106, X127, and X128 are absent; and wherein the IL-18R alpha binding moiety comprises (i) the amino acid sequence of QTFLHCFESRGIDWCIPWKS (SEQ ID NO: 177) or (ii) an amino acid sequence having 1, 2, 3, or 4 substitution(s), addition(s), and/or deletion(s) relative to the amino acid sequence of (i).

273. The polypeptide of any one of claims 261, wherein the IL-18R binding moiety comprises a first IL-18R alpha binding moiety and a second IL-18R alpha binding moiety.

274. The polypeptide of claim 273, wherein the first IL-18R alpha binding moiety comprises the amino acid sequence of
GGREDMECFWSPGQVWCIPSYLGG-CONH2 (SEQ ID NO: 403);
REDMECFWSPGQVWCIPSYL-CONH2 (SEQ ID NO: 405); or an amino acid sequence having 1, 2, 3, or 4 substitution(s), addition(s), and/or deletion(s) relative to SEQ ID NO: 403 or 405; and wherein the second IL-18R alpha binding moiety comprises the amino acid sequence of
YLDTCWVPFHYVNYLMCNVE (SEQ ID NO: 44);
REDMECFWSPGQVWCIPSYL (SEQ ID NO: 117);
REDMECFWSPGQVWCIPSYLGG (SEQ ID NO: 162);
YLDTCWVPFHYVNYLMCNVEGG (SEQ ID NO: 480); or an amino acid sequence having 1, 2, 3, or 4 substitution(s), addition(s), and/or deletion(s) relative to any one of SEQ ID NOs: 44, 117, 162, and 480.

275. The polypeptide of claim 273, wherein the polypeptide is in a N-N orientation, a N-C orientation, a C-N orientation, or a C-C orientation.

276. The polypeptide of claim 273, further comprising a linker between the first IL-18R alpha binding moiety and the second IL-18R alpha binding moiety, wherein the linker comprises SEQ ID NO: 401, SEQ ID NO: 401, formula I, formula II, formula III, formula IV, formula IV, or formula VI.

277. The polypeptide of claim 261, wherein the IL-18R binding moiety comprises a IL-18R alpha binding moiety and a IL-18R beta binding moiety.

278. The polypeptide of claim 277, wherein the IL-18R alpha binding moiety comprises the amino acid sequence of
GGREDMECFWSPGQVWCIPSYLGG-CONH2 (SEQ ID NO: 403);
REDMECFWSPGQVWCIPSYL-CONH2 (SEQ ID NO: 405);
GGREDMECFWSPGQVWCIPSYLGGSG-CONH2 (SEQ ID NO: 410);
GGREDMECFWSPGQVWCIPSYLGGSG (SEQ ID NO: 412);
REDMECFWSPGQVWCIPSYLGGS-CONH2 (SEQ ID NO: 419);
REDMECFWSPGQVWCIPSYLGGS (SEQ ID NO: 420); or an amino acid sequence having 1, 2, 3, or 4 substitution(s), addition(s), and/or deletion(s) relative to any one of SEQ ID NOs: 403, 405, 410, 412, 419, and 420; and wherein the IL-18R beta binding moiety comprises the amino acid sequence of
GGTSRLCWYFAGTVPGPCLEAIGG (SEQ ID NO: 224);
TSRLCWYFAGTVPGPCLEAI (SEQ ID NO: 253);
GGLCWYFGGTVRGPCLGG-CONH2 (SEQ ID NO: 404);
GGTSRLCWYFAGTVPGPCLEAIGG-CONH2 (SEQ ID NO: 407);
TSRLCWYFAGTVPGPCLEAI-CONH2 (SEQ ID NO: 408);
GGTSRLCWYFAGTVPGPCLEAI (SEQ ID NO: 409); or an amino acid sequence having 1, 2, 3, or 4 substitution(s), addition(s), and/or deletion(s) relative to any one of SEQ ID NOs: 224, 253, 404, 407, 408 and 409.

279. The polypeptide of claim 277, wherein the polypeptide is in a N-N orientation, a N-C orientation, a C-N orientation, or a C-C orientation.

280. The polypeptide of claim 277, further comprising a linker between the IL-18R alpha binding moiety and the IL-18R beta binding moiety, wherein the linker comprises SEQ ID NO: 401, SEQ ID NO: 401, formula I, formula II, formula III, formula IV, formula IV, or formula VI.

281. The polypeptide of claim 261, wherein the IL-18R binding moiety comprises an IL-18R alpha binding moiety and a peptide, wherein the IL-18R alpha binding moiety comprises the amino acid sequence of GGREDMECFWSPGQVWCIPSYLGG-CONH2 (SEQ ID NO: 403), REDMECFWSPGQVWCIPSYL-CONH2 (SEQ ID NO: 405), or an amino acid sequence having 1, 2, 3, or 4 substitution(s), addition(s), and/or deletion(s) relative to SEQ ID NO: 403 or 405; and wherein the peptide comprises the amino acid sequence of FEWTPGYWQPYALPLL (SEQ ID NO: 483) or an amino acid sequence having 1, 2, 3, or 4 substitution(s), addition(s), and/or deletion(s) relative to SEQ ID NO: 483.

282. The polypeptide of claim 261, wherein the IL-18R binding moiety is an IL-18R antagonist.

283. The polypeptide of claim 261, wherein the IL-18R binding moiety (a) binds to IL-18R alpha with a K_{D} of less than 10 μM, (b) has an IC50 of less than 10 μM, or a combination of (a) and (b).

284. The polypeptide of claim 261, wherein the IL-18R alpha binding moiety (a) does not bind to an IL-18BP or (b) binds to an IL-18BP with a KD that is at least 1,000-fold higher than the KD of the interaction between the IL-18R alpha binding moiety and IL-18R alpha.

285. A pharmaceutical composition comprising the polypeptide of claim 261 and a pharmaceutically acceptable excipient.

286. A method of treating a disease or disorder in a subject in need thereof, comprising administering to the subject the pharmaceutical composition of claim 285, wherein the disease or disorder comprises a cancer, an autoimmune disease, an inflammatory disease or disorder, an infectious disease or disorder, a metabolic disease or disorder, a neurodegenerative disease or disorder, a myocardial infarction, emphysema, psoriasis, a hemophagocytic syndrome, a macrophage activation syndrome, sepsis, acute kidney injury, or a combination thereof.
